(19) United States
(12) Patent Application Publication
(10) Pub. No.: US 2020/0171123 A1
(43) Pub. Date: Jun. 4, 2020

(54) COMPOSITIONS FOR USE IN THE TREATMENT OF ULCERATIVE COLITIS

(71) Applicant: Sublimity Therapeutics Limited, Dublin (IE)

(72) Inventor: Ivan Coulter, Co. Dublin (IE)

(73) Assignee: Sublimity Therapeutics Limited, Dublin (IE)

(21) Appl. No.: 16/780,700

(22) Filed: Feb. 3, 2020

Related U.S. Application Data
(63) Continuation of application No. 15/110,397, filed on Jul. 7, 2016.

(30) Foreign Application Priority Data
Jan. 10, 2014 (GB) 1400442.8

(51) Int. Cl. A61K 38/13; A61K 9/50; A61K 9/48; A61K 9/00; A61K 31/573; A61K 31/606; A61K 45/06

(52) U.S. Cl. CPC A61K 38/13; A61K 9/5026; A61K 9/4866; A61K 9/4858; A61K 9/0053; A61K 45/06; A61K 9/5036; A61K 31/606; A61K 9/5073; A61K 9/5047; A61K 31/573

(57) ABSTRACT

An oral modified release composition comprising cyclosporin, wherein the composition is for use in the treatment of ulcerative colitis in a patient, wherein the composition is for use in the concurrent treatment of the patient with an active agent selected from an aminosalicylate and a steroid, and a fixed or free combination thereof. Also claimed are kits comprising the oral modified release composition and the active agent. Also disclosed are methods for the treatment of ulcerative colitis using the oral modified release composition.

This invention relates to an oral modified release composition comprising cyclosporin, wherein the composition is for use in the treatment of ulcerative colitis. The composition may be used for the treatment of ulcerative colitis concurrently with other active agents. The invention also relates to kits comprising the composition and another active agent.

### BACKGROUND

Ulcerative colitis (UC) is an inflammatory disease that affects the lining of the colon and the rectum. The main symptom of the disease is constant diarrhoea mixed with blood and mucus. This is an intermittent disease with periods of exacerbated disease (flares) and periods that are relatively disease free. The symptoms can vary in severity and generally start gradually, these include: abdominal pain and sounds, fever, tenesmus, blood loss and weight loss. Although the symptoms of this disease can sometimes diminish spontaneously, usually treatment is required to induce remission.

UC affects approximately 2 million people worldwide. Although the cause of UC is unknown, both genetic and environmental factors are believed to be implicated in the aetiology of the disease.

UC is an autoimmune disorder and is generally treated using anti-inflammatory drugs. Currently there are no curative drug treatments of UC. Treatment aims to control flare-ups of the disease with the intention of inducing and maintaining remission. The type of treatment used depends on the severity of the disease. Guidelines for the treatment of ulcerative colitis are available and include “Ulcerative Colitis Practice Guidelines in Adults (Update): American College of Gastroenterology, Practice Parameters Committee” the American College of Gastroenterology 2004; and Dignass et al. “Second European evidence-based consensus on the diagnosis and management of ulcerative colitis: part 2: current management”, J Crohns Colitis. 2012 December; 6(10):991-1030. doi: 10.1016/j.crohns.2012.09.002. Epub 2012 Oct. 3; both of which are incorporated herein.

Aminosalicylates (ASAs) are used to manage symptoms and to induce remission in mild and moderate UC. Oral steroids (e.g. corticosteroids) are often prescribed for treatment of UC when ASAs alone are ineffective, and are the first line therapy to induce remission. In some instances acute severe flares may require intravenous corticosteroid therapy to induce a remission of the UC. Long-term use of corticosteroids for the treatment of UC is not recommended due to undesirable side-effects associated with the steroid use. However, in some patients the UC becomes steroid dependent and complete cessation of steroid treatment can result in a relapse and/or exacerbation of a flare. Steroids may be used alone or concurrently with administration of ASAs.

Subjects requiring either frequent or long term use of steroids may be prescribed other immunomodulators such as azathioprine (AZA) or mercaptopurine (6-MP), in conjunction with 5-ASAs, to maintain remission.

Subjects with active moderate or severe UC who fail to respond, or respond inadequately to other treatments such as those outlined above may be prescribed a biological agent, generally an antibody therapy such as an anti-TNFα biological treatment, for example infliximab or adalimumab (Rutgeerts et al. “Infliximab for induction and maintenance therapy for ulcerative colitis”, The New England Journal of Medicine, 2005, 353 (23): 2462-76; Reinisch et al “Adalimumab for induction of clinical remission in moderately to severely active ulcerative colitis results of a randomised controlled trial”. Gut 2012; 60:780-787). In general, anti-TNF agents are initiated to manage acute severe UC when other therapies fail to induce remission. However, biological anti-TNF treatments are only of limited effectiveness in the treatment of UC and many patients with severe UC do not remit and a number of patients that do remit develop resistance to the antibody therapies. Additionally the use of such biological agents may be associated with undesirable side effects including increased susceptibility to tuberculosis and other infections. Long term use of antibody therapy may also be associated with undesirable immunologic side-effects.

For subjects with severe UC which fails to respond to drug treatment surgical intervention may be required. Surgical treatment for UC includes colectomy, which involves the partial or complete removal of the large intestine.

Cyclosporin (or cyclosporine) is a cyclic polypeptide which has immunosuppressive and anti-inflammatory properties. The compound has been approved for the prevention of organ rejection following kidney, liver, heart, combined heart-lung, lung or pancreas transplantation, for the prevention of rejection following bone marrow transplantation; the treatment and prophylaxis of Graft Versus Host Disease (GVHD); psoriasis; atopic dermatitis, rheumatoid arthritis and nephrotic syndrome (Neoral™ Summary of Product Characteristics 24 Feb. 2012).

In the early 1990s, intravenous cyclosporin was shown to be effective as therapy for subjects with severe, steroid-refractory UC (Cohen, “Intravenous cyclosporine in severe ulcerative colitis: ready to stand alone?” Gastroenterology 2001; 120: 1541-1552; Lichtiger et al “Cyclosporin in severe ulcerative colitis refractory to steroid therapy”, N Engl J Med 1994; 330: 1841-1845).

Oral administration of a microemulsion of cyclosporin (Neoral™) has been used to treat chronic steroid-dependent severe UC subjects to reduce the steroid usage in the subjects. “Oral microemulsion cyclosporin to reduce steroids rapidly in chronic active ulcerative colitis.” European Journal of Gastroenterology & Hepatology 1999, 11(8):905-908). The patients in this trial had very severe chronic UC and were facing colectomy.

However, current commercially-available formulations of cyclosporin (intravenous [Sandimmun™] and immediate-release oral [for example, but not limited to Sandimmune™ and Neoral™]), exhibit undesirable side effects including nephrotoxic, hepatotoxic, and immunologic side effects (Dean et al Infliximab or cyclosporine for acute severe ulcerative colitis: a retrospective analysis.” J Gastroenterol Hepatol. 2012 March; 27(3):487-92).

Sandimmun™ is a solution of 50 mg/ml of cyclosporin in ethanol and polyethoxylated castor oil (for example Kolliphor™ EL). The product is also available as orally administered formulations, including, a soft gelatin capsule containing a solution of cyclosporin in ethanol, corn oil and lineoyl macrogolglycerides (Sandimmune™ Soft Gelatin capsules) and as an orally administered solution containing the cyclosporin dissolved in olive oil, ethanol, and labrafil M 1944 CS (polyethoxylated oleic glycerides) (Sandimmune™ Oral Solution). More recently a microemulsion concentrate formulation has been approved containing cyclosporin dissolved in DL-α-tocopherol, absolute ethanol, propylene glycol, corn oil-mono-di-triglycerides, polyoxyl 40 hydrogenated castor oil (Neoral™). Following oral administration the Neoral™ formulation results in the formation of a microemulsion and is stated to have an improved bioavailability compared to orally administered Sandimmune™. These orally administered cyclosporin compositions are all instant release compositions following administration of which cyclosporin will be present at high concentration in the stomach and small intestine from where it is systemically absorbed.

Sandborn et al. (J Clin Pharmacol. 1991; 31:76-80) determined the relative systemic absorption of cyclosporin following oral and intravenous as well as oil- and water-based enemas. Based on negligible plasma cyclosporin concentrations observed following enema administration, it was suggested that cyclosporin, even when solubilised, is poorly absorbed from the colon. The enemas however demonstrated considerable efficacy in the treatment of inflammatory bowel disease (Ranzi T, et al, Lancet 1989; 2:97). Intravenous or orally administered instant release compositions of cyclosporin such as Neoral™ in the treatment of inflammatory bowel disease is dose dependent, requiring high doses of cyclosporin to ensure adequate concentration reaches the colon. Systemic toxicity of cyclosporin is also known to be dose and duration dependent.

The undesirable side effects associated with the currently commercially available intravenous and oral cyclosporin formulations limits the duration for which such treatments can be used, generally for a maximum of about three or four months and such treatments are only suitable for the treatment of severe UC as a salvage therapy in patients facing surgical intervention. Additionally, use of currently available intravenous and oral cyclosporin formulations in combination with oral or intravenous steroids may further limit the duration for which such combined treatment could be used as a result of the combined negative side effects of the cyclosporin and the steroids.

WO 2008/122965 discloses oral cyclosporin minicapsule compositions which release cyclosporin in at least the colon. WO2010/133609 discloses compositions comprising a water-soluble polymer matrix in which are dispersed droplets of oil. The disclosed compositions also contain an active principle.

However, there remains a need for alternative treatments of ulcerative colitis. In particular there is a need for alternative treatments of moderate or severe ulcerative colitis as an alternative to using anti-TNF or other biological treatments and/or the use of currently available cyclosporin formulations.

The inventors have found that the use of an oral modified release composition comprising cyclosporin is beneficial in the treatment of ulcerative colitis. Particularly, use of an oral modified release composition comprising cyclosporin concurrently with an aminosalicylate and/or a steroid is beneficial in the treatment of ulcerative colitis.

### BRIEF SUMMARY OF THE DISCLOSURE

In accordance with the present invention there is provided an oral modified release composition comprising cyclosporin, wherein the composition is for use in the treatment of ulcerative colitis in a patient, wherein the composition is for use in the concurrent treatment of the patient with the cyclosporin and an active agent selected from an aminosalicylate, a steroid, and a fixed or free combination thereof.

Unless stated otherwise, references herein to “use of the composition” are to be understood as referring to the use of the modified release composition comprising cyclosporin concurrently with the active agent.

The active agent may be or comprise an aminosalicylate. The active agent may be or comprise a steroid. The active agent may be or comprise an aminosalicylate and a steroid.

The composition and the active agent may be in separate dosage forms for use in simultaneous, separate or sequential administration to the patient. Use of separate dosage forms is generally preferred because this enables more flexibility with respect to the individual dosages, the dosage regimen and the route of administration of the composition comprising cyclosporin and the active agent(s). However, optionally one or more of the active agent(s) may be included in the composition to provide a composition comprising cyclosporin and one or more of the active agents. For example, the composition may comprise cyclosporin and a steroid. The composition may comprise cyclosporin and an aminosalicylate. The composition may comprise cyclosporin, a steroid and an aminosalicylate. Where two or more of the said active agents are used in the concurrent treatment, the active agents may optionally be provided as a combined dosage form, for example a dosage form comprising an aminosalicylate and a steroid.

The modified release composition may be used to treat a patient with mild, moderate or severe ulcerative colitis. The patient may have mild ulcerative colitis. The patient may have moderate ulcerative colitis. The patient may have severe ulcerative colitis. The composition of the invention may therefore be used to treat patients with mild and/or moderate ulcerative colitis. Alternatively the composition may be used to treat patients with moderate to severe ulcerative colitis. Diagnosis of mild, moderate or severe ulcerative colitis may be determined using known methods, for example as described in the detailed description below.

Ulcerative colitis is characterised by acute exacerbations of symptoms or “flares” in the disease when the disease is active. Flares in the disease activity are generally interspersed with periods in which the disease is relatively stable when a patient will be symptom free or only experience mild symptoms.

The modified release composition for the use of the invention may be for use in the treatment of active ulcerative colitis, for example a flare of the disease.

The modified release composition for the use of the invention may be for use to induce a remission in a patient with active ulcerative colitis. For example to induce remission in a patient experiencing a flare in ulcerative colitis. For example to induce remission in a patient with mild, moderate or severe ulcerative colitis. The term “remission” is defined in the detailed description below. Suitably a patient may be considered to be in remission when the total Mayo score of the patient after treatment with the composition is 2 points or lower.

The modified release composition for the use of the invention may be used to achieve a clinical response in the patient such that the patient experiences a reduction in the symptoms of the ulcerative colitis. A “response” is defined in the detailed description and may for example be a reduction in the Mayo score of the patient, for example a reduction in the total Mayo score of 3 or more points compared with the total Mayo score at the start of the treatment. For example a clinical response may be one or more of the following symptom measures: mucosal healing, reducing rectal bleeding or reducing stool frequency. A clinical response may be an improvement in the physician's global assessment of the patient (an improvement in the global assessment score). Optionally a clinical response may be any one or more of the above symptom measures and an improvement in the physician's global assessment of the patient.

The modified release composition for the use of the invention may be used to induce mucosal healing in the patient. Accordingly the composition may be for mucosal healing of the colon of a patient with ulcerative colitis.

The oral modified release composition is suitably administered to the patient for a sufficient time to induce remission or a response in the patient. For example a patient may be administered the modified release composition for up to 4 weeks, up to 6 weeks up to 8 weeks or up to 12 weeks in order to induce a remission or a response of the ulcerative colitis. Suitably the oral modified release composition is administered to the patient for at least 1 week, at least 4 weeks, at least 6 weeks, at least 8 weeks or at least 12 weeks, for example from 1 week to 24 weeks, from 4 weeks to 24 weeks, form 6 weeks to 24 weeks, from 8 weeks to 24 weeks, from 12 weeks to 24 weeks. When the composition described anywhere herein is used as to provide induction treatment for ulcerative colitis, the composition may for example be administered to the patient for any of the time periods above. The dosages of cyclosporin suitable for induction treatment are as described herein. Suitably the composition is administered to a patient once or twice a day to during an induction treatment of ulcerative colitis.

The modified release composition for the use of the invention may be for use in a maintenance of remission treatment of ulcerative colitis.

Maintenance or remission treatment of ulcerative colitis may require the administration of the composition to a patient over a prolonged period of time to maintain the ulcerative colitis in remission. Accordingly the composition may be administered to the patient for months, years or even the life-time of the patient when used to provide a maintenance of remission treatment of ulcerative colitis. Accordingly a maintenance of remission treatment may be used for a period of at least 1 month, at least 6 months or at least 1 year, for example from about 6 months to about 5 years or longer (possibly for the life-time of the patient). The above treatment periods are suitable for use in any of the maintenance of remission treatment of ulcerative colitis described herein. Suitably the composition is administered to the patient once or twice per day during the maintenance treatment. However, it is possible that longer dosage frequencies periods may be suitable such as once per week to maintain remission of the ulcerative colitis.

Steroids are often used in the treatment of ulcerative colitis. For example a patient may be treated with one or more oral or rectally administered steroids. Patients may also require the administration of intravenous steroids, for example to treat a severe acute flare of the ulcerative colitis or to treat severe ulcerative colitis. Some patients treated with oral steroids, for example oral prednisolone or budesonide, may fail to respond to the oral steroid or may suffer a relapse. The ulcerative colitis in such patients remains active despite the use of oral steroids and is considered to be steroid refractory ulcerative colitis.

The modified release composition for the use of the invention may be for use in the treatment of steroid refractory ulcerative colitis. The steroid refractory ulcerative colitis may, for example, be moderate or severe steroid refractory ulcerative colitis. Particularly, the composition may be for use in the treatment of oral steroid refractory ulcerative colitis (i.e. ulcerative colitis which is refractory to orally administered steroids). For example, the composition may be for use in the treatment of moderate or severe oral steroid refractory ulcerative colitis.

In some patients receiving oral steroid treatment, the ulcerative colitis may be steroid dependent. This is to say that when the steroid dose administered to the patient is reduced or eliminated the patient will relapse and suffer increased symptoms or possibly an acute flare in the ulcerative colitis.

The modified release composition for the use of the invention may be for use in the treatment of steroid dependent ulcerative colitis, for example in the treatment of oral steroid dependent ulcerative colitis (ulcerative colitis dependent upon treatment with oral steroids). The steroid dependent ulcerative colitis may, for example, be moderate or severe steroid dependent ulcerative colitis, for example moderate or severe oral steroid dependent ulcerative colitis.

Patients with ulcerative colitis may be treated with a thiopurine immunosuppressant, for example selected from azathioprine or mercaptopurine (6-mercaptopurine or 6-MP). The modified release composition comprising cyclosporin may be used concurrently with the active agent as herein described (aminosalicylate and/or steroid), and in addition concurrently with a thiopurine immunosuppressant. Accordingly the composition may be used concurrently with an aminosalicylate and a thiopurine immunosuppressant. The composition may be used concurrently with a steroid and an aminosalicylate. The composition may be used concurrently with an aminosalicylate, a steroid and a thiopurine immunosuppressant.

Some patients treated with thiopurine immunosuppressants become refractory to the thiopurine, such that the ulcerative colitis is thiopurine refractory. The composition for the use of the invention may be for use in the treatment of ulcerative colitis wherein the ulcerative colitis is immunosuppressant refractory for example azathioprine refractory or mercaptopurine refractory. The composition may be for use in the treatment of thiopurine refractory ulcerative colitis, wherein the composition is for use in the concurrent treatment of the patient with the active agent.

The composition may suitably be for the use in the treatment of ulcerative colitis in a patient, wherein the composition is for use in the concurrent treatment of the patient with an active agent selected from an aminosalicylate, a steroid, and a fixed or free combination thereof; and wherein the patient is not treated with a thiopurine immunosuppressant.

Some ulcerative colitis patients treated with biological therapies for ulcerative colitis, such as an antibody therapy, become refractory (for example the patient fails to respond or becomes non-responsive after initial treatment) to the biological treatment (e.g. anti-TNF antibody or an integrin inhibitor antibody such as vedolizumab). The modified release composition for the use of the invention may be used to treat ulcerative colitis which is refractory to a biological treatment of ulcerative colitis. The composition of the invention may be particularly useful for the treatment of patients that initially respond to a biological therapy, but subsequently become non-responsive or resistant to further treatment with the biological treatment, due to, for example the development of autoantibodies to the biological treatment. A patient may be considered to have become refractory to a biological treatment when the patient has active ulcerative colitis or ulcerative colitis which relapses or flares in spite of being treated with the biological treatment for at least 1 week, 2 weeks, 1 month, 2 months or three months. Many such refractory patents have moderate to severe, and more often severe, ulcerative colitis wherein there are very few treatment options available other than surgery. The modified release composition comprising cyclosporin may therefore be used as a rescue therapy for the treatment of moderate to severe (particularly severe) ulcerative colitis which is refractory to a biological treatment for ulcerative colitis. The composition may be administered alone or suitably is for use in the concurrent treatment of the patient with the active agent (i.e. concurrently with an aminosalicylate, a steroid, or a fixed or free combination thereof).

Some ulcerative colitis patients treated with an anti-TNF antibody, (for example for example infliximab, adalimumab or golimumab) become refractory to the anti-TNF antibody, such that the ulcerative colitis is anti-TNF antibody refractory ulcerative colitis (i.e. the ulcerative colitis fails to respond or go into remission despite treatment with an anti-TNF antibody). The composition for the use of the invention may be for use in the treatment of anti-TNF antibody refractory ulcerative colitis wherein, wherein the composition is for use in the concurrent treatment of the patient with the active agent.

The modified release composition for the use of the invention may be used to treat ulcerative colitis which affects any part of the colon. For example, the ulcerative colitis may be left-sided colitis or may be extensive colitis, which affects substantially the whole or a significant part of the colon. In an embodiment the use according to the invention is for the treatment of ulcerative protosigmoiditis. In one embodiment the use according to the invention is for the treatment of left-sided colitis. In another embodiment of the invention the use is for the treatment of extensive colitis (pancolitis). In another embodiment the use according to the invention is for the treatment of UC limited to the rectum (ulcerative proctitis). In another embodiment the use according to the invention is not for the treatment of ulcerative proctitis. As mentioned herein, the treatment may be for mild, moderate or severe ulcerative colitis. For example, the use of the invention may be for the treatment of moderate or severe extensive colitis affecting any part of the colon.

The modified release composition may be for use in simultaneous, sequential or separate administration with the active agent(s) (i.e. the aminosalicylate and/or the steroid). Accordingly the modified release composition and said active agent are for use as separate dosage forms. Alternatively, the aminosalicylate and/or the steroid is/are included in the composition.

The aminosalicylate may be administered to the patient by any suitable route of administration. Optionally the aminosalicylate is orally administered or rectally administered to the patient. Optionally the aminosalicylate is orally administered to the patient.

The steroid may be administered to the patient by any suitable route of administration, for example orally administered, rectally administered or intravenously administered. Optionally the steroid is orally administered or intravenously administered. Optionally the steroid is orally administered or rectally administered. Optionally the steroid is orally administered.

Generally it is desirable to minimise steroid use when treating ulcerative colitis so as to minimise the undesirable side effects associated with steroid use. When the composition is used concurrently with an active agent which is or comprises a steroid, the dose of steroid may be reduced or tapered during the treatment of the patient as the patient responds to the treatment (for example by showing a clinical response or remission of the ulcerative colitis). For example the daily steroid dose may be gradually reduced over a period of time until the steroid is eliminated from the treatment regimen.

Accordingly the modified release composition may be for use in the treatment of ulcerative colitis in a patient, wherein the patient is treated concurrently with the cyclosporin and the active agent, wherein the active agent is or comprises (i) an aminosalicylate and a steroid; or (ii) a steroid; wherein the composition is for use in a dosage regimen wherein:

the patient is administered a first dosing regimen comprising the composition and (i) the aminosalicylate and the steroid or (ii) the steroid; and one or more subsequent dosage regimen comprising the composition and (i) the aminosalicylate and the steroid; or (ii) the steroid; and wherein

the total daily dose of the steroid in the first dosage regimen is greater than the total daily dose of the steroid in at least one of the subsequent dosage regimen.

Suitably the subsequent dosage regimen comprises two or more treatment cycles comprising the concurrent treatment of the patient with the composition, a steroid and optionally an aminosalicylate, wherein the daily dose of steroid administered to the patient is reduced after the completion of each treatment cycle; optionally wherein each treatment cycle is from about 1 week to about 12 weeks, for example about 1 week to about 8 weeks or about 1 week to about 4 weeks in duration.

The composition may be used to provide an induction and maintenance of remission treatment for ulcerative colitis. Suitably the composition is used to provide both induction of remission and maintenance of remission of ulcerative colitis. During the induction treatment the composition is administered concurrently with the active agent(s) to induce a remission of ulcerative colitis. The patient is then administered with the composition to maintain remission of ulcerative colitis symptoms. During the maintenance treatment the agent may be administered concurrently with for example an aminosalicylate. Optionally during the maintenance treatment the composition may be administered to the patient alone without concurrent administration of the agent. Where the maintenance treatment comprises the modified release composition administered concurrently with an aminosalicylate, the dose of aminosalicylate may be reduced during the maintenance treatment to minimise the drug loading administered to the patient whilst maintaining the ulcerative colitis in remission. Optionally the dose of aminosalicylate may be completely eliminated from the maintenance treatment such that the patient is treated only with the modified release composition comprising cyclosporin. For example the daily dose of aminosalicylate may be reduced after each treatment cycle of the maintenance therapy, optionally until the aminosalicylate is eliminated, wherein each treatment cycle of the maintenance treatment is from about 1 week to about 12 weeks, for example about 1 week to about 8 weeks or about 1 week to about 4 weeks in duration.

Accordingly the composition may be for use in the treatment of ulcerative colitis in a patient, wherein the patient is treated concurrently with the cyclosporin and the active agent, wherein the active agent is or comprises (i) an aminosalicylate and a steroid; or (ii) a steroid or (iii) an aminosalicylate; wherein the composition is for use in a dosage regimen wherein:

the patient is administered at least one initial dosage regimen comprising the composition and (i) an aminosalicylate and a steroid; or (ii) a steroid or (iii) an aminosalicylate; and one or more subsequent dosage regimen comprising the composition alone or the composition and an aminosalicylate, wherein the subsequent dosage regimen does not comprise a steroid. Optionally the initial dosage regimen may comprise the composition, and a steroid. Optionally the initial dosage regimen may comprise the composition, a steroid and an aminosalicylate.

The composition may be used alone for use in a maintenance of remission treatment of ulcerative colitis, for example to maintain remission of ulcerative colitis.

According to another aspect of the invention there is provided an oral modified release composition comprising cyclosporin, wherein the composition is for use in a maintenance of remission treatment of ulcerative colitis in a patient wherein the ulcerative colitis is in remission. In this aspect the composition may be administered the patient alone to the patient to maintain the ulcerative colitis in remission. Optionally the modified release composition comprising cyclosporin may be administered to the patient concurrently with another drug suitable for use as a maintenance of remission treatment of ulcerative colitis. Suitably the patient is not treated with a steroid as part of the maintenance or remission treatment. Accordingly the composition may be for use in a steroid-free maintenance of remission treatment of ulcerative colitis.

The composition may be for use in the treatment of a patient whose ulcerative colitis is treated exclusively with the composition and with one or both of an aminosalicylate and a steroid; optionally wherein the patient is treated exclusively with the composition and an aminosalicylate; optionally wherein the patient is treated exclusively with the composition and a steroid; optionally wherein the patient is treated exclusively with the composition, a steroid and an aminosalicylate.

The composition may be for use in the treatment of ulcerative colitis as described herein wherein the patient is not treated with azathioprine or 6-mercaptopurine. Optionally therefore the composition is not used for the treatment of ulcerative colitis concurrently with azathioprine or 6-mercaptopurine.

The composition may be for use in the treatment of ulcerative colitis as described herein wherein the patient is not treated with an antibody therapy for ulcerative colitis, for example the patient is not treated with an anti-TNF antibody for example infliximab, adalimumab or golimumab. For example the patient is not treated with an integrin inhibitor antibody such as vedolizumab.

Optionally a patient with ulcerative colitis may be treated with the active agent described herein for at least 14 days prior to administering the oral modified release composition comprising cyclosporin.

Also provided is an oral modified release composition comprising cyclosporin, wherein the composition is for use in administration alone, or concurrently with an active agent selected from an aminosalicylate, a steroid and a free or fixed combination thereof, the composition being for use in the treatment of:

(a) moderate or severe active ulcerative colitis in a patient, wherein the patient is non-responsive or intolerant to prior treatment with one or more of an aminosalicylate, a steroid, azathioprine or 6-mercaptopurine; and/or

(b) moderate or severe active steroid dependent ulcerative colitis in a patient; and/or

(c) moderate or severe ulcerative colitis to reduce the signs and symptoms of ulcerative colitis; and/or

(d) moderate or severe ulcerative colitis to induce mucosal healing; and/or

(e) moderate or severe ulcerative colitis to induce remission and optionally maintain the ulcerative colitis in remission; and/or

(f) moderate or severe ulcerative colitis in a patient wherein the patient is non-responsive to prior treatment with a biological therapy for ulcerative colitis, for example an anti-TNF antibody therapy (e.g. infliximab, adalimumab or golimumab) or an integrin inhibitor antibody therapy (e.g. vedolizumab).

Optionally in this aspect, the composition is for use concurrently with the said active agent.

In this embodiment the composition may be used in the treatment of any one of (a) to (f), or any combination of two or more thereof. In this embodiment the treatment in (f) may induce remission of ulcerative colitis and provide a maintenance of remission treatment as described herein. Optionally in this embodiment an oral modified release composition is for use in administration alone, or concurrently with an active agent selected from an aminosalicylate, a steroid and a free or fixed combination thereof, the composition being for use in the treatment of any of conditions (a) to (e). Suitably in conditions (a) and (f) above the patient is refractory to the prior treatment. The composition for use in (f) may be particularly useful in a patient that initially responds to the biological therapy, but subsequently develops resistance to the biological therapy and becomes refractory to the biological therapy.

Reference to reducing the signs or symptoms herein includes for example one or more of the clinical responses described herein.

Also provided is an oral modified release composition comprising cyclosporin, wherein the composition is for use in the treatment of moderate ulcerative colitis in a patient. In this aspect the moderate ulcerative colitis is suitably treated with the composition alone.

Also disclosed is a method for treating ulcerative colitis in a patient being treated with at least one of (i) an aminosalicylate and (ii) a steroid, the method comprising orally administering to the patient a therapeutically effective amount of a modified release composition comprising cyclosporin.

Also disclosed is method for treating ulcerative colitis, the method comprising administering to a patient in need thereof a therapeutic amount of (a) an oral modified release composition comprising cyclosporin; and (b) at least one of (i) an aminosalicylate and (ii) a steroid, wherein the modified release composition, the aminosalicylate and the steroid are administered simultaneously, sequentially or separately.

Also disclosed is a method for treating ulcerative colitis, the method comprising orally administering to a patient in need thereof a therapeutically effective amount of a modified release composition comprising cyclosporin concurrently with administration to the patient of a therapeutically effective amount of at least one of (i) an aminosalicylate and (ii) a steroid.

Also disclosed is a method for treating ulcerative colitis, the method comprising selecting a patient with ulcerative colitis that is being treated with at least one of (i) an aminosalicylate and (ii) a steroid, and orally administering to the patient a therapeutically effective amount of a modified release composition comprising cyclosporin.

Also disclosed is a method for treating ulcerative colitis in a patent that is or has been treated with at least one of (i) an aminosalicylate or (ii) a steroid, the method comprising orally administering to the patient a therapeutically effective amount of a modified release composition comprising cyclosporin.

Also disclosed is a method for the treatment of:

(a) moderate or severe active ulcerative colitis in a patient, wherein the patient is non-responsive or intolerant to prior treatment with one or more of an aminosalicylate, a steroid, azathioprine or 6-mercaptopurine; and/or

(b) moderate or severe active steroid dependent ulcerative colitis in a patient; and/or

(c) moderate or severe ulcerative colitis to reduce the signs and symptoms of ulcerative colitis; and/or

(d) moderate or severe ulcerative colitis to induce mucosal healing; and/or

(e) moderate or severe ulcerative colitis to induce remission and optionally maintain the ulcerative colitis in remission; and/or

(f) moderate or severe ulcerative colitis in a patient wherein the patient is non-responsive to prior treatment with a biological therapy for ulcerative colitis, for example an anti-TNF antibody therapy (e.g. infliximab, adalimumab or golimumab) or an integrin inhibitor antibody therapy (e.g. vedolizumab);

the method comprising administering to the patient a therapeutically active amount of an oral modified release composition comprising cyclosporin, wherein, in the method of treatment, the composition is administered alone or in combination with administration to the patient of a therapeutically effective amount of at least one of (i) an aminosalicylate and (ii) a steroid.

Suitably in this aspect the oral modified release composition comprising cyclosporin is administered to the patient in combination with administration to the patient of a therapeutically effective amount of at least one of (i) an aminosalicylate and (ii) a steroid.

In this embodiment the method of treatment may be any one of (a) to (f), or any combination of two or more thereof.

Suitably in conditions (a) and (f) above the patient is refractory to the prior treatment. For example in (f) the patient initially responds to the biological therapy but subsequently develops resistance to the therapy and becomes refractory to the biological treatment.

Suitably in the methods of treatment disclosed herein the oral modified release composition comprising cyclosporin and the at least one of (i) an aminosalicylate and (ii) a steroid are administered to the patient simultaneously, sequentially or separately. In this embodiment the treatment in (e) may induce remission of ulcerative colitis and provide a maintenance of remission treatment as described herein.

Also disclosed is a method for the maintenance treatment of ulcerative colitis, the method comprising orally administering to a patient with ulcerative colitis in remission a therapeutically effective amount of a modified release composition comprising cyclosporin.

Also disclosed is a method for treating moderate ulcerative colitis, the method comprising orally administering to a patient in need thereof a therapeutically effective amount of a modified release composition comprising cyclosporin.

Also disclosed is the use of an oral modified release composition comprising cyclosporin in the manufacture of a medicament for use in the treatment of ulcerative colitis in a patient, wherein the composition is for use in the concurrent treatment of the patient with the cyclosporin and an active agent selected from an aminosalicylate, a steroid, and a fixed or free combination thereof.

Suitably the oral modified release composition comprising cyclosporin is administered to the patient to provide a total daily dose of cyclosporin of from about 1 mg to about 500 mg, optionally a total daily dose of from about 10 mg to about 500 mg, from about 10 mg to about 250 mg, from about 30 mg to about 150 mg, from about 10 to about 20 mg, from about 10 mg to about 25 mg, from about 10 mg to about 50 mg, from about 10 mg to about 100 mg form about 10 mg to about 150 mg, from about 35 to about 40 mg, from about 25 mg to about 250 mg, for example a total daily dose of cyclosporin of about 1 mg, about 5 mg, about 10 mg, about 15 mg, about 20 mg, about 25 mg, about 30 mg, about 35 mg, about 37.5 mg, about 40 mg, about 50 mg, about 70 mg, about 75 mg, about 100 mg, about 105 mg, about 112.5 mg, about 125 mg, about 140 mg, about 150 mg, about 175 mg, about 187.5 mg, about 200 mg, about 210 mg, about 225 mg, about 245 mg, about 250 mg, about 262.5 mg, about 280 mg, about 300 mg, about 315 mg, about 337.5, about 350 mg, about 375 mg, about 385 mg, about 412.5 mg, about 420 mg, about 450 mg, about 455 mg, about 487.5 mg, about 490 mg or about 500 mg.

Optionally the oral modified release composition comprising cyclosporin is administered to the patient to provide a total daily dose of cyclosporin of from 0.1 mg/kg to about 10 mg/kg; for example from about 0.5 mg/kg to about 5 mg/kg, from about 0.5 mg/kg to about 2 mg/kg or about 0.5 mg/kg to about 1.5 mg/kg, for example about 1 mg/kg.

The oral modified release composition may be administered to the patient as a single daily dose, alternatively the daily dose may be administered as a split dose of for example two, three, four or more doses of the modified release composition per day. Suitably the modified release composition is administered to the patient once or twice daily in any of the treatments for ulcerative colitis described herein.

By way of example, a suitable once daily oral dosing regimen of the modified release composition provides a single daily dose of 75 mg cyclosporin. However, other doses may be administered for example 37.5 mg or 150 mg once per day. An example of a twice daily dosage regimen may be 37.5 mg twice per day, 75 mg twice per day or 150 mg twice per day.

Suitably the patient is a human; optionally wherein the patient is an adult human; optionally wherein the patient is a human aged less than 18 years.

Also provided is a kit comprising (a) an oral modified release composition comprising cyclosporin; and (b) at least one of an aminosalicylate and a steroid. Optionally in the kit the oral modified release composition comprising cyclosporin; and the at least one of an aminosalicylate and a steroid are for use in the treatment of ulcerative colitis in a patient as described in any of the embodiments herein. Suitably the aminosalicylate is present in the kit in the form of a composition comprising the aminosalicylate. Suitably the steroid is present in the kit in the form of a composition comprising the steroid. The kit may further comprise instructions for the concurrent treatment of a patient with ulcerative colitis, wherein the treatment is as described herein.

Suitably the aminosalicylate is selected from a 5-aminosalicylate and a 4-aminosalicylate, or a prodrug, or a pharmaceutically acceptable salt thereof, for example the aminosalicylate acid is a 5-aminosalicylate or a prodrug or a pharmaceutically acceptable salt thereof, for example selected from mesalazine, sulfasalazine, olsalazine, ipsalazide, balsalazide and benzalazine, or a pharmaceutically acceptable salt thereof.

Suitably the steroid is a corticosteroid optionally in the form of a pharmaceutically acceptable salt or ester, for example selected from aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide and triamcinolone alcohol.

Optionally the steroid, for example budesonide, is administered to the patient in the form of a modified release composition comprising the steroid; suitably wherein the composition releases the steroid in at least the colon.

The oral modified release composition comprising cyclosporin provides release of cyclosporin into at least the colon following oral administration of the composition. Suitably the cyclosporin is released from the composition in an active form (for example a solubilised form as discussed below in relation to the composition) at the required location within the lower GI tract, particularly in the colon. The local release of cyclosporin directly into the colon provides cyclosporin locally within the colon, the site of diseased tissue in patients with ulcerative colitis. However, cyclosporin is poorly absorbed by the colon and therefore release of the cyclosporin in the colon enables the cyclosporin to interact with the diseased tissue whilst minimising systemic absorption of the drug. By minimising release of cyclosporin in the upper GI tract, systemic absorption of the cyclosporin, and associated side effects are minimised. Oral administration of the modified release composition of the invention provides high levels of cyclosporin in the colon and enables a relatively low dose of cyclosporin to be administered to a patient, compared to for example oral or IV administration of cyclosporin using currently available cyclosporin formulations such as Neoral™ and Sandimmun™. Generally high doses of Neoral™ or Sandimmun™ of the order 4 mg/kg to 8 mg/kg are required to achieve a therapeutic benefit in UC patients. The oral modified release composition of the invention may enable a relatively low dose of cyclosporin to be administered, therefore further reducing undesirable side effects, whilst providing a therapeutic benefit for the treatment of ulcerative colitis resulting from the local release of cyclosporin in the colon and the relatively high levels of exposure of the colonic tissue to cyclosporin.

Suitably the composition releases no or only small amounts of cyclosporin in the upper GI tract, particularly in the stomach, duodenum and small intestine so as to minimise systemic absorption of the cyclosporin. The modified release composition comprising cyclosporin suitably releases less than 20% (suitably less than 10%) of the cyclosporin after 2 hours; and releases at least 50% of the cyclosporin after 12 hours, when measured in a two stage dissolution test using a USP Apparatus II with a paddle speed of 75 rpm and a dissolution medium temperature of 37° C.; wherein for the first 2 hours of the dissolution test the dissolution medium is 750 ml of 0.1 N HCl, and at 2 hours 250 ml of 0.2M tribasic sodium phosphate containing 2% SDS is added to the dissolution medium and the pH is adjusted to pH 6.8 (i.e. volume of dissolution medium in the second part of the test is 1000 ml). (Hereafter referred to as “the two stage dissolution test).

In one embodiment the composition releases 0 to 10% of the cyclosporin after 2 hours; and releases from 60 to 100% of the cyclosporin after 12 hours, when measured in the two stage dissolution test. In another embodiment the composition releases less than 20% of the cyclosporin after 2 hours; releases 10 to 40% of the cyclosporin after 4 hours and releases at least 50% of the cyclosporin after 12 hours, when measured in the two stage dissolution test. In a further embodiment the composition releases less than 20% of the cyclosporin after 2 hours; releases 15 to 40% of the cyclosporin after 4 hours; and releases at least 75% of the cyclosporin after 12 hours, when measured in the two stage dissolution test. In a further embodiment the composition releases less than 10% of the cyclosporin after 2 hours; releases 10 to 30% of the cyclosporin after 4 hours; and releases at least 60% of the cyclosporin after 12 hours, when measured in the two stage dissolution test. In a further embodiment the composition releases from about 50 to about 75% of the cyclosporin between 4 hours and 12 hours in the two stage dissolution test, for example the composition releases from about 55 to about 75%, particularly from about 55 to 70% of the cyclosporin between 4 hours and 12 hours in the two stage dissolution test. In a further embodiment the composition releases less than 15% (for example less than 10%, suitably from 0 to 10%) of the cyclosporin after 2 hours; releases 10% to 40% (for example 10% to 30%, 20% to 35%, or 25% to 35%) of the cyclosporin after 4 hours; and releases from about 30% to 80% (for example 55% to 70%) of the cyclosporin between 4 hours and 12 hours, when measured in the two stage dissolution test.

In another embodiment the composition releases 0 to 10% of the cyclosporin after 2 hours; and releases from 50 to 100% of the cyclosporin after 12 hours, when measured in the two stage dissolution test. In another embodiment the composition releases less than 20% of the cyclosporin after 2 hours; releases 5 to 40% of the cyclosporin at 4 hours and releases at least 50% of the cyclosporin at 12 hours, when measured in the two stage dissolution test.

In a further embodiment the composition releases less than 20% of the cyclosporin after 2 hours; releases 10 to 40% of the cyclosporin at 4 hours; and releases at least 60% of the cyclosporin at 12 hours, when measured in the two stage dissolution test. In a further embodiment the composition releases less than 10% of the cyclosporin after 2 hours; releases 10 to 30% of the cyclosporin at 4 hours; and releases at least 50% of the cyclosporin at 12 hours, when measured in the two stage dissolution test. In a further embodiment the composition releases from about 30 to about 75% of the cyclosporin between 4 hours and 12 hours in the two stage dissolution test, for example the composition releases from about 40 to about 75%, particularly from about 45 to 70% of the cyclosporin between 4 hours and 12 hours in the two stage dissolution test.

In another embodiment the composition releases less than 15% (for example 0 to 10%) of the cyclosporin after 2 hours; releases 10% to 40% (for example 10% to 35%, or suitably 15% to 35%) of the cyclosporin at 4 hours; and releases from about 25% to 70% (for example 40% to 70%) of the cyclosporin between 4 hours and 12 hours in the two stage dissolution test.

In one embodiment the composition of the invention releases less than 15% (for example 0 to 10%) of the cyclosporin after 2 hours; releases 10% to 30% of the cyclosporin after 4 hours; releases 30 to 50% of the cyclosporin after 6 hours; releases 60 to 80% of the cyclosporin after 12 hours.

Suitably the composition releases at least 80%, at least 85%, at least 90%, at least 95% or at least 99% of the cyclosporin within 24 hours, when measured in the two stage dissolution test. Accordingly the compositions described herein release at least 80%, at least 85%, at least 90% or at least 95% of the cyclosporin within 24 hours, when measured in the two stage dissolution test.

It is to be understood that the in-vitro release profiles described in the embodiment above are applicable to each of the embodiments described above or below. It is also to be understood that reference herein to “release” or “releases” of certain amounts of cyclosporin refers to the total amount of cyclosporin released from the composition into the dissolution medium of the two stage dissolution test at a given time point after the start of the dissolution test (i.e. when the composition is placed into the dissolution medium). By way of example reference to release of less than 10% cyclosporin after 2 hours, release of 10 to 30% cyclosporin after or at 4 hours and release of 30 to 50% cyclosporin after or at 6 hours means that less than 10% of the total amount of cyclosporin initially in the composition (the initial amount) is present in the dissolution medium at 2 hours; 10 to 30% of the initial amount of cyclosporin is in the dissolution medium at 4 hours and 30 to 50% of the initial amount of cyclosporin is in the dissolution medium at 6 hours.

The composition may comprise a matrix and cyclosporin. For example wherein the matrix is or comprises a polymer matrix comprising a polymer selected from a water-permeable polymer, a water-swellable polymer, a water-soluble polymer, a hydrogel forming polymer and a biodegradable polymer. In a particular embodiment the matrix is or comprises a hydrogel forming polymer matrix.

The composition may comprise a coating to control or modulate release of the cyclosporin from the composition (a modified release coating). Advantageously the coating is a polymeric coating to provide delayed and/or sustained release of the cyclosporin form the composition. Suitable such modified release coatings are described in more detail below under “Modified Release Coatings) and includes a coating which is or comprises a coating selected from a controlled release polymer, a sustained release polymer, an enteric polymer, a pH independent polymer, a pH dependent polymer and a polymer specifically susceptible to degradation by bacterial enzymes in the gastrointestinal tract, or a combination of two or more such polymers. In a particular embodiment the coating is or comprises a pH-independent polymer, for example a coating which is or comprises ethyl cellulose. In a further specific embodiment the coating is or comprises a pH-independent polymer, for example ethyl cellulose and a water-soluble polysaccharide, for example selected pectin or chitosan, or a combination thereof, particularly pectin.

It has been found that compositions comprising cyclosporin which are coated with a sub-coat which is or comprises a water-soluble cellulose ether or a water-soluble derivative of a cellulose ether prior to coating with a further modified release coating as described above provides advantageous properties. In particular it has been found that the presence of a sub-coating results in a higher total release of cyclosporin from the composition and/or a greater rate of release of the cyclosporin compared to a composition which does not have a sub-coat. In vitro dissolution testing has also shown that the sub-coated compositions according to the invention reduce batch to batch variability in the in-vitro release profile. Accordingly, the sub-coated compositions may demonstrate a reduced inter and/or intra-patient variability compared to non-sub coated compositions.

According to an embodiment of the invention the composition comprising cyclosporin further comprises a first coating and a second coating outside the first coating; and wherein:

the first coating is or comprises a water-soluble cellulose ether or a water-soluble derivative of a cellulose ether; and

the second coating is or comprises a coating, suitably a polymeric coating, as defined above to control or modulate release of cyclosporin from the composition. The first and second coatings are suitably coatings on a core comprising cyclosporin.

Accordingly the first coating is a sub-coating as described herein and the second coating is suitably a modified release coating as described herein. The first and second coatings are suitably different polymers.

The first coating suitably is or comprises a water-soluble cellulose ether or a water-soluble ester of a cellulose ether. Particularly the first coating is or comprises a water-soluble cellulose ether. The water-soluble cellulose ether may for example be a water-soluble cellulose ether selected from an alkyl cellulose; a hydroxyalkyl cellulose; a hydroxyalkyl alkyl cellulose; and a carboxyalkyl cellulose. Suitably the first coating is or comprises one or more water-soluble cellulose ethers selected from methyl cellulose, hydroxyethyl cellulose, hydroxypropyl cellulose and hydroxypropylmethyl cellulose and combinations thereof. In particular embodiments the first coating is or comprises a water-soluble hydroxypropylmethyl cellulose. The water-soluble cellulose ethers and water-soluble derivatives thereof (e.g. water-soluble esters of a cellulose ether) present in the first coating (sub-coat) suitably form at least 40%, 50%, 60%, 70%, 80%, 85% or 90% by weight of the dry weight of the first coating.

The coating(s) described above may be applied to a core comprising a hydrogel forming polymer and cyclosporin. Accordingly in an embodiment the composition comprises a core and the coating is outside the core, wherein the core comprises a water-soluble polymer matrix and cyclosporin.

In a further embodiment the composition comprises a core, a first coating outside the core, wherein the first coating is a water-soluble cellulose ether or a water-soluble derivative thereof as described above; and a second coating outside the first coating, wherein the core comprises a hydrogel forming polymer matrix and cyclosporin. Suitably the first coating is or comprises a water-soluble cellulose ether, for example HPMC.

In particular embodiments the core has the form of a solid colloid, the colloid comprising a continuous phase and a disperse phase, wherein the continuous phase comprises a hydrogel forming polymer matrix. Suitable continuous phases and disperse phases which may be used to form the core are defined in more detail below and in the detailed description of the invention.

Suitably the continuous phase of the core is or comprises a hydrogel forming polymer matrix. In embodiments the hydrogel forming polymer matrix is or comprises a hydrocolloid, a non-hydrocolloid gum or chitosan. In a particular embodiment the a hydrogel forming polymer matrix is or comprises gelatin, agar, a polyethylene glycol, starch, casein, chitosan, soya bean protein, safflower protein, alginates, gellan gum, carrageenan, xanthan gum, phthalated gelatin, succinated gelatin, cellulosephthalate-acetate, oleoresin, polyvinylacetate, hydroxypropyl methyl cellulose, polymerisates of acrylic or methacrylic esters and polyvinylacetate-phthalate and any derivative of any of the foregoing; or a mixture of two or more such polymers. In a further embodiment the hydrogel forming polymer matrix is or comprises a hydrocolloid selected from carrageenan, gelatin, agar and pectin, or a combination thereof optionally selected from gelatin and agar or a combination thereof. Particularly, the polymer of the hydrogel forming polymer matrix is or comprises gelatin. In an embodiment, the hydrogel-forming polymer does not comprise a cellulose or a cellulose derivative, e.g. does not comprise a cellulose ether.

In embodiments cyclosporin is or is comprised in the disperse phase of the core.

The disperse phase may be solid, semi-solid or liquid. In particular, the disperse phase may be liquid. In other particular instances the disperse phase may be semi-solid, for example it may be waxy.

The disperse phase may be a hydrophobic phase, for example a hydrophobic phase which is a solid, a semi-solid or a liquid. Suitably the disperse phase comprises a hydrophobic excipient and optionally a solvent miscible therewith, optionally wherein the cyclosporin is soluble in the disperse phase.

The cyclosporin may be dissolved in the disperse phase. The cyclosporin may be suspended in the disperse phase. The disperse phase may be as described elsewhere herein, for example it may be as described in the immediately preceding two paragraphs.

Accordingly the disperse phase may further comprise a solvent, wherein the solvent is miscible with the disperse phase and water, optionally wherein the solvent is selected from 2-(2-ethoxyethoxy)ethanol and a poly(ethylene glycol), particularly wherein the solvent is 2-(2-ethoxyethoxy)ethanol. The solvent may also be or comprise a poly(ethylene glycol) selected from a PEG with an average molecular weight of from about 200 to about 400, for example PEG 200 or PEG 400.

In a particular embodiment the disperse phase comprises a liquid lipid and a solvent, wherein the solvent is miscible with the liquid lipid and water, optionally wherein the solvent is selected from 2-(2-ethoxyethoxy)ethanol and a poly(ethylene glycol), particularly wherein the solvent is 2-(2-ethoxyethoxy)ethanol. In a further embodiment the disperse phase comprises an oil phase comprising a medium chain mono- di- or triglyceride (particularly a medium chain triglyceride), a polyethoxylated castor oil and 2-(ethoxyethoxy)ethanol.

In embodiments the composition comprises one or more surfactants, suitable surfactants are described in more detail in the detailed description of the invention. In those embodiments where the composition comprises a core in the form of a solid colloid, the colloid comprising a continuous phase and a disperse phase, wherein the continuous phase comprises a hydrogel forming polymer matrix, surfactant may be present in the continuous phase, the disperse phase or both the continuous phase and the disperse phase. Accordingly in one embodiment the core comprises a surfactant present in at least the continuous phase, the surfactant having an HLB value of from about 1 to about 15. In another embodiment the core comprises a surfactant present in at least the continuous phase, the surfactant having an HLB value of greater than 10, for example greater than 20, for example from about 10 to about 15. In a further embodiment the disperse phase comprises a surfactant with an HLB value in the range of from 1 to 10, for example from 1 to 5.

In one embodiment the composition comprises a core and a coating outside the core, wherein the core is in the form of a solid colloid, the colloid comprising a continuous phase and a disperse phase, wherein the disperse phase is or comprises:

cyclosporin;

a medium chain mono- di- or tri-glyceride, for example caprylic/capric triglyceride;

a non-ionic surfactant (for example a polyethoxylated castor oil); and

a solvent (for example 2-(ethoxyethoxy)ethanol);

and wherein the continuous phase is or comprises:

a hydrogel forming polymer matrix which is or comprises a hydrocolloid selected from carrageenan, gelatin, agar and pectin, or a combination thereof optionally selected from gelatin and agar or a combination thereof, more optionally the polymer of the water-soluble polymer matrix is or comprises gelatin;

optionally a plasticiser, for example a plasticiser selected from glycerin, a polyol for example sorbitol, polyethylene glycol and triethyl citrate or a mixture thereof, particularly sorbitol; and

an anionic surfactant, for example at least one surfactant selected from fatty acid salts, alkyl sulphate salts and bile salts, particularly an alkyl sulphate salt, for example sodium dodecyl sulfate; and wherein the coating on the core is any of the coatings described herein. Suitably the coating comprises a first coating and a second coating outside the first coating; and wherein

the first coating is or comprises a water-soluble cellulose ether or a water-soluble derivative of a cellulose ether as described above (for example the first coating is or comprises a water soluble cellulose ether as described herein, particularly HPMC); and

the second coating is or comprises a coating, suitably a polymeric coating, as defined above to control or modulate release of cyclosporin from the composition.

In embodiments comprising a first coating and a second coating, for example as mentioned above and in the detailed description. A particular first coating is or comprises hydroxypropylmethyl cellulose and a particular second coating outside the first coating is or comprises a pH independent polymer, for example ethyl cellulose; more particularly the second coating is or comprises ethyl cellulose and optionally a polysaccharide selected from water-soluble and naturally occurring polysaccharides, for example pectin or another water-soluble naturally occurring polysaccharide. The second coating may therefore contain pectin or another said polysaccharide or it may be substantially free of pectin and other said polysaccharides. There are therefore disclosed second coatings which comprise ethylcellulose as a modified release polymer and which further comprise pectin or another said polysaccharide as well as second coatings which comprise ethylcellulose as a modified release polymer and which do not further comprise pectin or another said polysaccharide.

The core of the composition described above may comprise a hydrogel forming polymer matrix and cyclosporin and have the characteristics of a core obtained by a process comprising:

(i) dissolving a hydrogel forming polymer in an aqueous liquid to form a solution;
(ii) dissolving or dispersing cyclosporin in a liquid to form a solution or dispersion (particularly a solution) of the cyclosporin in the liquid;
(iii) mixing the aqueous solution (i) and the solution or dispersion (ii) to form a colloid;
(iv) ejecting the colloid through a nozzle to form droplets;
(v) causing or allowing the a hydrogel forming polymer to gel or solidify to form a hydrogel-forming polymer matrix, whereby the droplets become minibeads; and
(vi) drying the minibeads.

Suitably the aqueous phase pre-mix (i) further comprises an anionic surfactant, e.g. as described elsewhere herein, for example sodium dodecyl sulfate (SDS).

The solution or dispersion (ii) (oil phase) may be prepared by dissolving or dispersing the cyclosporin in a suitable hydrophobic liquid. The hydrophobic liquid may be for example, any of the oils or liquid lipids described herein. By way of example the hydrophobic liquid may be, or comprise, saturated or unsaturated fatty acids or a triglyceride, or an ester or ether thereof with polyethylene glycols. A particular oil for the oil phase is or comprises a triglyceride, for example an oil comprising a medium chain triglyceride, optionally wherein the oil comprises a triglyceride of at least one fatty acid selected from fatty acids having 6, 7, 8, 9, 10, 11 or 12 carbon atoms, e.g. C₈-C₁₀ fatty acids.

It has been found that the use of certain surfactants during the manufacture of the compositions are particularly effective in stabilising the colloid (for example emulsion), resulting from the mixing of the mixing the aqueous solution (i) and oil phase (ii) comprising the cyclosporin. When the colloid comprises an oil-in-water emulsion, it has been found that the presence of a surfactant having an HLB of up to 10 (particularly up to 8) in the oil phase is particularly effective in stabilising the emulsion during the preparation of the composition. The presence of such surfactants has been found to inhibit the formation of cyclosporin crystals after the formation of the colloid (oil-in-water emulsion). The presence of a surfactant with an HLB of up to 10 maintains the cyclosporin in solution in the oil phase during manufacture and may also provide favourable release of the cyclosporin in a solubilised form from the composition following oral administration of the composition to a subject. Compositions comprising a surfactant with an HLB of up to 10 in at least the oil phase may exhibit high rates of release and/or extent of release of cyclosporin from the composition compared to the use of surfactants with a higher HLB value in the oil phase. The presence of a surfactant with an HLB of up to 10 in at least the oil phase in the composition may inhibit precipitation of cyclosporin after release of the cyclosporin from the composition thereby retaining higher levels of cyclosporin in a solubilised form within the GI tract, for example in the colon, thereby enhancing the colonic lumen and tissue bioavailability of active cyclosporin.

Accordingly the orally administered modified release composition may comprise a core having the form of a solid colloid, the colloid comprising a continuous phase being or comprising a hydrogel forming polymer and a disperse phase being or comprising cyclosporin, and an oil phase, the oil phase comprising an oil and one or more surfactants, wherein the oil and the surfactant have an HLB of up to 10, for example an HLB in the range 0-10.

The surfactant present in the oil phase may be any of the surfactants described herein with an HLB value up to 10 The surfactant present in the oil phase may a HLB value selected from: up to 8, up to 7, 1-8, 1-7, 1-5, 2-5, 1-4, 1-3, 1-2, 2-4, 3-4, 5-8, 6-8 and 6-7. Suitably the surfactant present in the oil phase is a non-ionic surfactant having an HLB value above.

The oil may be any of the oils described herein. Suitably the oil is not itself a surfactant. However, certain oils, particularly those derived from natural sources will comprise components which may have surface active properties. For example many triglyceride oils also comprise mono and diglyceride components and may therefore exhibit some surfactant like properties. Accordingly the oil suitably has an HLB value of 0-10, however suitably the oil has an HLB which is close to 0 for example an HLB of 0 to 3, optionally about 0, about 1 or about 2.

The oil and the surfactant present in the oil phase may both independently have an HLB value of 0 to 10. The oil may have an HLB of 1-5 and the surfactant may have an HLB of 2-8, optionally 3-7, 2-6, or 3-4. Suitably the oil and the surfactant are different.

The cyclosporin may be soluble in the oil. The cyclosporin may be soluble in the surfactant used in the oil phase. Suitably the cyclosporin is soluble in both the oil and the surfactant. Suitably, substantially all of the cyclosporin may be dissolved in the oil phase.

The oil phase may further comprises a solvent, wherein the solvent is miscible with the disperse phase and water, optionally wherein the solvent is selected from 2-(2-ethoxyethoxy)ethanol and a poly(ethylene glycol), particularly wherein the solvent is 2-(2-ethoxyethoxy)ethanol.

The hydrogel forming polymer of the core may be any of the hydrogel forming polymers described herein.

The composition may further comprise additional surfactants in addition to the surfactant present in the oil phase. In particular the continuous phase comprising the hydrogel forming polymer may further comprise one or more surfactants. Surfactants which may be present in the continuous phase are any of the surfactants described herein as being suitable for inclusion in the aqueous (continuous) phase of the composition. Suitably the continuous phase comprises one or more anionic surfactant, for example at least one surfactant selected from fatty acid salts, alkyl sulfates and bile salts, particularly the surfactant in the continuous phase is or comprises an alkyl sulfate, for example sodium dodecyl sulfate. In one embodiment the core having the characteristics of a core obtained by the process above is a core comprising a hydrogel forming polymer matrix and a non-aqueous phase dispersed in the a hydrogel forming polymer matrix, wherein the core is or comprises gelatin, SDS, sorbitol, polyethoxylated castor oil, caprylic/capric triglyceride, 2-(ethoxyethoxy)ethanol; wherein the aqueous solution (i) is or comprises gelatin, sorbitol and SDS; and the solution or dispersion (ii) is or comprises polyethoxylated castor oil, caprylic/capric triglyceride, 2-(ethoxyethoxy)ethanol and cyclosporin.

In the embodiments above cores having the characteristics of cores obtained by the process are coated, optionally with a first sub-coating, and with a coating to control or modify release, preferably a polymeric coating as described above and herein to provide the modified release composition for the use according to the invention. The coated core may be obtained by applying to the core the coating, e.g. applying to the core the first and second coatings as described above. Before the coating is applied, the core may be made by a process having steps (i) to (vi) described above. Suitable methods for applying the coating(s) are described below and include applying the coatings by spray coating a coating composition onto the core.

In an embodiment the composition is in the form of a minibead. Suitably the largest cross sectional dimension of the minibead is from 0.1 to 5 mm, for example from 1 mm to 5 mm as in the case of from 1 mm to 3 mm or 1 mm to 2 mm. The minibead may be spheroidal. The spheroidal minibead may have an aspect ratio of no more than 1.5, for example of from 1.1 to 1.5.

Administration to a patient of a dosage form comprising multiple minibeads in for example a capsule (e.g. a hard or soft gelatin capsule of an HPMC capsule) will result in the release of multiple minibeads from the capsule into the stomach when the capsule dissolves or disintegrates. The small size of the minibeads means that individual minibeads can quickly pass through the pyloric sphincter and into the GI tract, thereby minimising the transit time from the stomach into the lower GI tract compared to the administration of for example large dosage forms such as enteric coated tablets or capsules. Administering the composition to a patient in the form of multiple minibeads allows for a distribution of individual minibeads along a substantial length the colon as the minibeads transit along the GI tract. Release of cyclosporin from the individual minibeads distributed along the colon, provides direct exposure of large sections of colonic tissue to the cyclosporin released from the individual minibeads.

The composition may be formulated into a unit dosage form for oral administration comprising from about 0.1 mg to about 1000 mg, optionally from about 1 mg to about 500 mg, from about 5 mg to about 30 mg, from about 10 mg to about 25 mg, from about 10 mg to about 50 mg, from about 10 mg to about 20 mg, from about 30 mg to about 40 mg, from about 35 mg to about 40 mg, from about 10 mg to about 300 mg, from about 10 mg to about 150 mg, from about 10 mg to about 100 mg, from about 25 mg to about 250 mg, from about 25 mg to 225 mg, about 1 mg, about 5 mg, about 10 mg, about 15 mg, about 20 mg, about 25 mg, about 30 mg, about 35 mg, about 37.5 mg, about 40 mg, about 50 mg, about 70 mg, about 75 mg, about 100 mg, about 105 mg, about 112.5 mg, about 125 mg, about 140 mg, about 150 mg, about 175 mg, about 187.5 mg, about 200 mg, about 210 mg, about 225 mg, about 245 mg, about 250 mg, about 262.5 mg, about 280 mg, about 300 mg, about 315 mg, about 337.5, about 350 mg, about 375 mg, about 385 mg, about 412.5 mg, about 420 mg, about 450 mg, about 455 mg, about 487.5 mg, about 490 mg or about 500 mg cyclosporin.

Suitably the composition is in a multiple minibead unit dosage form selected from soft or hard gel capsules, gelatin capsules, HPMC capsules, compressed tablets or sachets. The minibeads may be as described elsewhere herein.

Suitably the cyclosporin in the modified release composition of the invention is cyclosporin A.

### BRIEF DESCRIPTION OF THE DRAWINGS

Embodiments of the invention are further described hereinafter with reference to the accompanying drawings, in which:

FIG. 1 is a chart showing the percentage of subjects in clinical remission in the intention to treat population;

FIG. 2 is a chart showing the percentage of subjects in clinical response in the intention to treat population;

FIG. 3 is charts showing the percentage of subjects in clinical remission (FIG. 3a) and clinical response (FIG. 3b) in subjects with moderate ulcerative colitis;

FIG. 4 is charts showing the percentage of subjects in clinical remission (FIG. 4a) and clinical response (FIG. 4b) in subjects with mild-moderate ulcerative colitis being medicated concurrently with 5-ASA only;

FIG. 5 is charts showing the percentage of subjects in clinical remission (FIG. 5a) and clinical response (FIG. 5b) in subjects with moderate ulcerative colitis being medicated concurrently with 5-ASA only;

FIG. 6 is a chart showing Δ between CyCol® and placebo for response rate (Δ_{Res}) in the patient subgroups identified in Examples 2 to 5.

FIG. 7 shows the percentage of subjects achieving a clinical response in populations receiving placebo or an approved therapy for UC, as well as the Δ_{Res} for these therapies; and

FIG. 8 is charts showing the percentage of subjects in clinical remission (FIG. 8a) and clinical response (FIG. 8b) in subjects with mild-moderate ulcerative colitis and being medicated concurrently with 5-ASA and steroids.

FIG. 9 illustrates the screening, treatment and follow-up periods of the clinical trial protocol described in Example 11.

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

For the avoidance of doubt, it is hereby stated that the information disclosed earlier in this specification under the heading “Background” is relevant to the invention and is to be read as part of the disclosure of the invention.

The reader's attention is directed to all papers and documents which are filed concurrently with or previous to this specification in connection with this application and which are open to public inspection with this specification, and the contents of all such papers and documents are incorporated herein by reference.

The composition and the active agent(s) of the invention are administered to a patient concurrently. The term “concurrently” used herein includes administering the composition and the active agent(s) simultaneously, sequentially or separately to the patient. The composition and the active agent(s) may be administered simultaneously in a single formulation for example a tablet or a capsule or they may be administered simultaneously or nearly simultaneously in different dosage forms. The composition and the active agent(s) may be administered to the patient sequentially, wherein the composition is administered to the patient followed by the active agent(s); or vice versa, wherein one or more of the active agent is administered followed by administration of the composition. Separate administration includes administering the composition and the active agent(s) to the patient at different times. Where the composition and the active agent(s) are administered separately it is to be understood that the timing of separate dosing is selected such that the beneficial effect of the first administered composition/agent is not lost prior to administration of the second or further agent/composition. The time between administration of the composition/agent when separate administration is used will depend upon the specific agent(s), the composition, the dose, and the severity and/or extent of the ulcerative colitis. For example, the time between separate administration may be less than 1 minute, or up to 10 minutes, up to 30 minutes, up to 1 hour, up to 2 hours, up to 4 hours up to 6 hours up to 8 hours, up to 12 hours, up to 1 day, up to 1 week or up to 2 weeks. Where two or more active agents are administered as well as the composition, each active agent may be administered simultaneously, sequentially or separately with each other active agent.

The composition is orally administered to the patient. The active agent(s) may be administered by any suitable route to the patient for example orally, rectally or parenterally (e.g. intravenously). Where there more than one active agent is used in the use of the invention they may be administered to the patient by the same route or by different routes. For example one active agent (e.g. an aminosalicylate) may be administered orally and a second active agent (e.g. a steroid) may be administered intravenously. Alternatively both active agents could be administered orally.

The term “treatment”, and the therapies encompassed by this invention, include the following and combinations thereof: (1) reducing the risk of or inhibiting, e.g. delaying, initiation and/or progression of, a state, disorder or condition; (2) preventing, e.g. reducing the risk of, or delaying the appearance of clinical symptoms of a state, disorder or condition developing in a patient (e.g. human or animal) that may be afflicted with or predisposed to the state, disorder or condition but does not yet experience or display clinical or subclinical symptoms of the state, disorder or condition; (3) inhibiting the state, disorder or condition (e.g., arresting, reducing or delaying the development of the disease, or a relapse thereof in case of maintenance treatment, of at least one clinical or subclinical symptom thereof); and/or (4) relieving the condition (e.g. causing regression of the state, disorder or condition or at least one of its clinical or subclinical symptoms). Where the formulation of the invention is used in the treatment of a patient, treatment contemplates any one or more of: maintaining the health of the patient; restoring or improving the health of the patient; and delaying the progression of the disorder. The benefit to a patient to be treated may be either statistically significant or at least perceptible to the patient or to the physician. It will be understood that a medicament will not necessarily produce a clinical effect in every patient to whom it is administered, and this paragraph is to be understood accordingly.

Reference herein to a “therapeutically effective amount” is an amount sufficient to reduce or completely alleviate symptoms or other detrimental effects of a disorder; reverse, completely stop, or slow the progress of a disorder; or reduce the risk of a disorder getting worse; for example an amount sufficient to induce remission of ulcerative colitis, or an amount sufficient to maintain a remission of ulcerative colitis. It is further within the skill of one of ordinary skill in the art to determine appropriate treatment duration, appropriate doses, and any potential combination treatments, based upon an evaluation of therapeutic or prophylactic response.

Reference to “modified release” herein includes compositions which alter the release of a drug from the composition, particularly compositions which for example provide controlled release, extended (or sustained) release or delayed release or any combination thereof, for example delayed and controlled release of a drug from a composition following oral administration.

Reference to a “pharmaceutically acceptable salt” of a compound means a salt that is pharmaceutically acceptable and that possesses the desired pharmacological activity of the parent compound. Such pharmaceutically-acceptable salts may be for example, an acid-addition salt of a compound, for example an acid-addition salt with an inorganic or organic acid such as hydrochloric, hydrobromic, sulfuric, trifluoroacetic, citric or maleic acid; or, for example, a salt of a compound which is sufficiently acidic, for example an alkali or alkaline earth metal salt such as a sodium, calcium or magnesium salt, or an ammonium salt, or a salt with an organic base such as methylamine, dimethylamine, trimethylamine, piperidine or morpholine.

Reference to “a pro-drug” herein means a compound that is broken down in the human or animal body to release a compound in-vivo. A pro-drug may be used to alter the physical properties and/or the pharmacokinetic properties of a compound. A pro-drug can be formed when a compound contains a suitable group or substituent to which a property-modifying group can be attached. Examples of pro-drugs include in vivo cleavable ester derivatives that may be formed at a carboxy group or a hydroxy group in a compound and in-vivo cleavable amide derivatives that may be formed at a carboxy group or an amino group in a compound. Various forms of pro-drug have been described, for example in the following documents: Design of Pro-drugs, edited by H. Bundgaard, (Elsevier, 1985); A Textbook of Drug Design and Development, edited by Krogsgaard-Larsen; and H. Bundgaard, Chapter 5 “Design and Application of Pro-drugs”, by H. Bundgaard p. 113-191 (1991).

### Ulcerative Colitis

Ulcerative colitis is a chronic inflammatory disease characterised by diffuse mucosal inflammation of the colon. The disease is characterised by amongst other features bloody diarrhoea, often with symptoms of rectal urgency and tenesmus. The term “ulcerative colitis” used herein includes diverticulitis, pouchitis, proctitis, mucositis, diversion colitis, ischemic colitis, infectious colitis, chemical colitis, radiation-induced colitis, microscopic colitis (including collagenous colitis and lymphocytic colitis), atypical colitis, pseudomembraneous colitis, fulminant colitis, autistic enterocolitis, interdeminate colitis, Behcet's disease, jejunoiletis, ileitis, ileocolitis and granulomatous colitis. The invention contemplates the use of the composition as described herein for the treatment of any of such condition. Also contemplated is use of the composition for the uses described herein for use in the treatment of colitis associated with inflammatory diseases of the gastrointestinal tract, particularly colitis associated with inflammatory diseases affecting mainly the colon; for example colitis associated with primary sclerosing cholangitis or radiation.

When UC is suspected in a patient initial diagnosis generally includes a complete blood count to check for anaemia, urinalysis, stool culture, erythrocyte sedimentation rate (ESR) as an indicator of inflammation, liver and renal function tests, and electrolyte studies. However, these markers alone may not be sufficient to definitively diagnose ulcerative colitis. Suitably, therefore endoscopy is generally the most accurate diagnostic tool for UC. A flexible sigmoidoscopy is usually sufficient to diagnose UC, however, a full colonoscopy may be performed if diagnosis is unclear. This procedure involves an investigation for the presence of superficial ulceration, erythema or friability of the mucosa, loss of vascular appearance of the colon, and pseudopolyps.

Biopsies may also be taken in order to differentiate UC from Crohn's disease. The biopsy samples are generally taken at the time of endoscopy and are examined for distortion of crypt architecture, inflammation of the crypts, crypt abscesses, and haemorrhage or inflammation in the lamina propia.

The ulcerative colitis may affect part of the colon, or substantially the entire colon. The ulcerative colitis may be ulcerative proctosigmoiditis. Reference herein to “ulcerative proctosigmoiditis” refers to ulcerative colitis limited to the rectum and sigmoid colon.

The ulcerative colitis may be left-sided ulcerative colitis. Reference to “left-sided colitis” herein means ulcerative colitis which is limited to the proportion of the colon distal to the splenic flexure, more particularly ulcerative colitis that extends beyond the rectum and as far proximally as the splenic flexure.

The ulcerative colitis may be extensive ulcerative colitis wherein substantially all of the colon is affected. Reference to “extensive ulcerative” or “pancolitis” herein means ulcerative colitis which extends proximal to the splenic flexure (i.e. extending beyond the splenic flexure towards the ileo-caecal junction).

Accordingly, the composition of the invention may be used concurrently with the active agent for use in the treatment of ulcerative colitis that affects any part or substantially the whole of the colon, for example ulcerative colitis selected from ulcerative proctosigmoiditis, left-sided ulcerative colitis and extensive ulcerative colitis.

Ulcerative colitis is generally further characterised by the severity of the disease and may be mild, moderate or severe ulcerative colitis. Accordingly, the composition of the invention may be used concurrently with the active agent for use in the treatment of mild, moderate or severe ulcerative colitis. For example, the use of the composition in accordance with the invention may be for use in the treatment of mild ulcerative colitis. The use of the composition in accordance with the invention may be for use in the treatment of moderate ulcerative colitis. The use of the composition in accordance with the invention may be for use in the treatment of severe ulcerative colitis. The use of the composition in accordance with the invention may be for use in the treatment of patients with mild or moderate ulcerative colitis. The use of the composition in accordance with the invention may be for use in the treatment of patients with moderate or severe ulcerative colitis.

The severity of the ulcerative colitis may be determined by known methods, which generally rely upon a combination of patient characteristics. For example mild, moderate or severe UC may be determined as described in Dignas et al “Second European evidence-based consensus on the diagnosis and management of ulcerative colitis: Definitions and diagnosis”, J. Crohns Colitis. 2012 December; 6(10), which is incorporated herein by reference. Mild, moderate and severe ulcerative colitis may also be defined according to the criteria adopted by Truelove and Witts; Cortisone in ulcerative colitis; final report on a therapeutic trial. Br Med J 1955; 2:1041-8.

Accordingly mild ulcerative colitis is associated with fewer than 4 bowel movements per day. Moderate ulcerative colitis is associated with 4 or more bowel movements per day and may be further distinguished from mild ulcerative colitis by the presence of mucosal friability (bleeding on light contact with the rectal mucosa at sigmoidoscopy). Severe ulcerative colitis is associated with 6 or more bowel movements per day with blood in the stool and with systemic involvement, for example an ESR >30 mm/hour or CRP >30 mg/L. Accordingly patients with moderate UC are those between the mild and severe categories; namely patients with 4 or more bowel movements per day but without significant systemic involvement, for example ESR ≤30 mm/hour and CRP ≤30 mg/L. More particularly mild, moderate and severe ulcerative colitis may be defined according to the parameters set out in the table below adapted from Truelove and Witts:

| | Mild | | Moderate | | Severe | |
|---|---|---|---|---|---|---|
| Bloody | <4 | | 4 or more if | | ≥6 and | |
| stools/day | | | | | | |
| Pulse | <90 | bpm | ≤90 | bpm | >90 | bpm or |
| Temperature | <37.5° | C. | ≤37.8° | C. | >37.8° | C. or |
| Haemoglobin | >11.5 | g/dL | ≥10.5 | g/dL | <10.5 | g/dL or |
| ESR | <20 | mm/h | ≤30 | mm/h | >30 | mm/h or |

| | | | | | |
|---|---|---|---|---|---|
| or CRP | Normal | ≤30 | mg/L | >30 | mg/L |

The severity of ulcerative colitis may also be classified using an appropriate disease activity index (DAI). Such DAIs are well known. For example a DAI suitable for classifying ulcerative colitis is the Mayo score for ulcerative colitis (Schroeder et al; Coated oral 5-aminosalicylic acid therapy for mildly to moderately active ulcerative colitis. A randomized study; N Engl J Med. 1987 Dec. 24; 317(26):1625-9. PMID: 3317057). The Mayo scoring system is a 12 point composite index that is composed of inputs from the patient and from the person treating the patient, for example a physician. Each sub-score of the Mayo system ranges from 0 to 3 depending upon the severity. The sum of the individual sub-scores provides the total Mayo score. The Mayo DAI scoring system is described in the table below. The scoring system described in the table below may be used to determine the Mayo score/DAI score mentioned for any of the embodiments described herein.

| | | | | |
|---|---|---|---|---|
| Normal | Mild | Moderate | Severe | |
| (Score = 0) | (Score = 1) | (Score = 2) | (Score = 3) | Score |

| | | | | |
|---|---|---|---|---|
| Rectal Bleeding | None | Streaks of blood | Obvious blood | Mostly blood |
| Stool | Normal | 1-2/day > normal | 3-4/day > normal | >4/day > normal |
| Frequency* | | | | |
| Mucosal | Normal | Erythema | Marked erythema | Ulceration |
| Appearance/ | | Decreased | Friability | Spontaneous |
| Endoscopy | | vascular pattern | Granualarity | bleeding |
| Score | | Minimal granularity | Absent vascular | |
| | | | pattern | |
| | | | Bleeding or | |
| | | | minimal trauma | |
| | | | No ulceration | |
| Physician's | Normal | Mild | Moderate | Severe |
| Global | | | | |
| Assessment+ | | | | |

TOTAL DAI Score

*Stool frequency, ‘normal’ refers to the normal number of stools per day when the patient is in remission
+Physician's Global Assessment is based on rectal bleeding, stool frequency, mucosal appearance, patient reported abdominal pain, the patient's general sense of well-being and, physical examination findings.

The patient reported scores for Rectal Bleeding and Stool Frequency may be calculated from a daily diary using data recorded from the three days preceding the physician's assessment/clinic visit. The Mucosal Appearance Score may be based on the flexible sigmoidoscopy examination. The Physician Global Assessment (PGA) score will be based on sigmoidoscopy results, patient interviews and physical examinations.

In one embodiment use of the composition in accordance with the invention may be for use in the treatment of a patient with a total Mayo score of 6 or more, for example a total Mayo score of from 6 to 12. More particularly the composition may be used to treat a patient with a total Mayo score of from 6 to 12, and with a mucosal appearance score of 2 or more at baseline. As will be understood, reference to “baseline” herein is a reference to the condition of the patient before being treated with the composition.

In a further embodiment the use of the composition in accordance with the invention may be for use in the treatment of a patient with a combined daily stool frequency and rectal bleeding Mayo score of 4 or more at baseline.

In a further embodiment the use of the composition in accordance with the invention may be for use in the treatment of a patient with a combined daily stool frequency and rectal bleeding Mayo score of 4 or more at baseline, and with a mucosal appearance score of 2 or more at baseline.

In a further embodiment the use of the composition in accordance with the invention may be for use in the treatment of a patient with a total Mayo score of from 4 to 10, with a mucosal sub-score of ≥1.

Use of the composition in accordance with the invention may be for use to induce a remission of ulcerative colitis. The ulcerative colitis may be any of mild, moderate or severe ulcerative colitis and may also be ulcerative colitis affects any part or substantially all of the colon as described hereinbefore.

The term “remission” as used herein means a stool frequency ≤3/day with no visible blood in the stool. Suitably remission results in complete resolution of symptoms and mucosal healing, which may be determined by endoscopic examination. It is possible to further qualify a remission by reference to a suitable symptom scoring method, for example the Mayo scoring system. For example remission of ulcerative colitis may also be defined to be a total Mayo score of 2 points or less and with no individual sub-score exceeding 1.

The composition and active agents are used concurrently in accordance with the invention for a sufficient time to induce a remission of the ulcerative colitis. Generally induction of a remission will require treatment of the patient for a few weeks as described herein, for example for at least 4 weeks, at least 6 weeks, at least 8 weeks or at least 12 weeks, optionally from 1 week to 12 weeks. For example the patient may be treated for 1, 2, 3, 4, 5, 6, 7, 8, 9, 10, 11, or 12 weeks (or longer if required) to induce remission of the ulcerative colitis.

Use of the composition in accordance with the invention may be for use in the treatment of ulcerative colitis to provide a clinical response to the treatment. A clinical response means clinical and endoscopic improvement, depending (for the purpose of clinical trials) on the disease activity index used. In general, this means a decrease in disease activity index (DAI) of 30% or more, plus a decrease in the rectal bleeding and endoscopy sub-scores. A suitable DAI for the purposes of ulcerative colitis is the Mayo index as described above. Accordingly a clinical response as used herein may be defined as a decrease from baseline in the total Mayo score of 3, or a 30% reduction from the baseline Mayo score, and wherein patients treated have an accompanying decrease in the Mayo sub-score for rectal bleeding of at least 1 point compared to the baseline score or an absolute sub-score for rectal bleeding of 0 or 1.

Use of the composition in accordance with the invention may be for use in the treatment of ulcerative colitis to provide mucosal healing in the patient. Reference to “mucosal healing” herein means a reduction in endoscopy DAI score of at least 1 point, or an absolute DAI sub-score for endoscopy of 0 or 1.

Use of the composition in accordance with the invention may be for use in the treatment of ulcerative colitis to reduce rectal bleeding.

Use of the composition in accordance with the invention may be for use in the treatment of ulcerative colitis to reduce stool frequency.

Reference to “active” ulcerative colitis as used herein means the ulcerative colitis is biologically active. Patients with active disease are symptomatic and will exhibit one or more sign or symptom of ulcerative colitis, for example, rectal bleeding, increased stool frequency, mucosal inflammation or abnormal laboratory tests (for example for example elevated ESR or CRP values or decreased haemoglobin). A patient is considered in remission when the ulcerative colitis is not active.

Reference to ulcerative colitis being “refractory” to a particular therapy means ulcerative colitis which is active or which relapses or flares in spite of being treated with that therapy.

A randomized, double-blind, placebo-controlled study of a modified release composition according to the invention in the treatment of mild to moderate ulcerative colitis is described in the Examples herein. The trial was carried out over a 4 week treatment period and despite the relatively short duration of the trial clinical efficacy and remission effects were observed in the treated patients compared to those receiving placebo. Patients treated concurrently with the composition and the agent in accordance with the invention showed improved response and remission compared to placebo. Particularly patients with mild or moderate ulcerative colitis treated concurrently with the composition and a 5-aminosalicylate or a 5-aminosalicylate and a steroid showed beneficial response and remission effects compared to patients that were not treated with the composition.

Accordingly in one embodiment of the invention there is provided an oral modified release composition comprising cyclosporin, wherein the composition is for use in the treatment of mild or moderate ulcerative colitis in a patient, wherein the composition is for use in the concurrent treatment of the patient with the cyclosporin and an active agent selected from a 5-aminosalicylate, a steroid, and a fixed or free combination thereof. Suitably in this embodiment the agent is a 5-aminosalicylate. Alternatively the agent is a steroid and a 5-aminosalicylate. Suitably in this embodiment treatment induces a remission of mild or moderate ulcerative colitis. Alternatively in this embodiment the treatment of the mild or moderate ulcerative colitis results in a clinical response. Alternatively in this embodiment the treatment of the mild or moderate ulcerative colitis results in mucosal healing of the colon.

The clinical trial discussed above also showed that patients with moderate ulcerative colitis treated concurrently with the composition and an aminosalicylate or an aminosalicylate and a steroid in accordance with the invention showed beneficial response and remission rates compared to patients that were not treated with the composition (i.e. patients treated with the agent alone). More particularly patients with moderate ulcerative colitis treated concurrently with the composition and an aminosalicylate (suitably a 5-aminosalicylate) showed significant response and remission compared to patients that did not receive the composition. As discussed in the Examples below, the difference in response/remission between patients treated concurrently with the composition and the active agent according to the invention compared to patients treated with the active agent alone (the Δ) were comparable to response and remission Δ's observed in clinical trials carried out using antibody therapies such as infliximab or adalimumab in patients with moderate to severe ulcerative colitis. The comparable Δ values were observed despite the fact that the antibody clinical trials were carried out for longer (6 to 8 weeks) compared to the short 4 week trial using the composition of the present invention).

Accordingly, there is provided an oral modified release composition comprising cyclosporin, wherein the composition is for use in the treatment of moderate ulcerative colitis in a patient, wherein the composition is for use in the concurrent treatment of the patient with the cyclosporin and an active agent selected from an aminosalicylate and a steroid, and a fixed or free combination thereof. Suitably in this embodiment the agent is or comprises an aminosalicylate, more particularly the agent is or comprises a 5-aminosalicylate. Still further the agent is an aminosalicylate, more particularly a 5-aminosalicylate.

Patients with ulcerative colitis may experience a flare in ulcerative colitis. Particularly a patient with UC in clinical remission may experience a relapse or flare in the UC. Such flares may occur spontaneously or in response to certain treatments administered to the patient.

The terms “flare” or “relapse” used herein refer to an increase in symptoms of the UC, for example increased stool frequency, increased with rectal bleeding and/or appearance of abnormal mucosa evidenced by sigmoidoscopy. Depending on the severity of the flare the patient may experience a flare resulting in mild, moderate or severe ulcerative colitis, wherein mild, moderate and severe ulcerative colitis are as hereinbefore defined.

The composition may be used in accordance with the invention for use to treat a flare of ulcerative colitis, for example a flare which results in mild, moderate or severe ulcerative colitis and wherein the ulcerative colitis may affect any part or substantially all of the colon.

The composition for the use of the invention may be for use in the treatment of steroid refractory ulcerative colitis.

The term “Steroid refractory ulcerative colitis” means patients which have active ulcerative colitis despite being treated with a steroid. For example patients which have active ulcerative colitis despite being treated with prednisolone up to 0.75 mg/kg/day over a period of 4 weeks.

The composition may be used for the treatment of a patient refractory to orally administered steroids, for example in a patient with mild, moderate or severe (for example moderate or severe and particularly severe) ulcerative colitis which is refractory to orally administered steroids. The active agent used concurrently with the composition is suitably a steroid or a steroid and an aminosalicylate. The steroid of the agent may for example be administered to the patient orally or intravenously. In one aspect the steroid of the agent is administered orally. In another aspect the steroid of the agent is administered to the patient intravenously.

The composition may be used for the treatment of a patient refractory to intravenously administered steroids, for example in a patient with moderate or severe (for example severe) ulcerative colitis which is refractory to intravenously administered steroids. The active agent used concurrently with the composition is suitably a steroid or a steroid and an aminosalicylate. The steroid of the agent may be administered to the patient orally or intravenously. In one aspect the steroid of the agent is administered orally. In another aspect the steroid of the agent is administered to the patient intravenously. The term “ulcerative colitis which is refractive to intravenously administered steroids” includes ulcerative colitis patients wherein the ulcerative colitis fails to respond to intravenously administered steroids, for example patients which fail to respond to intravenously administered steroids administered to the patient for a period of seven days or more. For example the patient does not show a clinical response or the ulcerative colitis does not go into remission despite the intravenous administration of a steroid.

Concurrent use of composition and a steroid, particularly an intravenously administered steroid, may be advantageous for use in the treatment of moderate or severe (particularly severe) ulcerative colitis refractory to intravenous steroids. Patients with ulcerative colitis refractory to intravenously administered steroids currently have limited treatment options available as an alternative to surgery. Currently steroid refractory ulcerative colitis may be treated with an antibody therapy, for example a TNFα antibody therapy including infliximab or adalimumab. The use of the composition concurrently with the agent in accordance with the invention may induce a remission or response in the ulcerative colitis thereby avoiding the need for treatment with TNFα antibody therapy or surgery.

The composition for the use of the invention may be for use in the treatment of steroid dependent ulcerative colitis. The ulcerative colitis may be mild, moderate or severe, particularly moderate or severe steroid dependent ulcerative colitis.

The term “steroid dependent ulcerative colitis” means ulcerative colitis which is being treated with a steroid, for example an orally administered steroid, wherein the ulcerative colitis relapses (or flares) when the steroid dose is reduced or stopped. Thus patients steroid dependent ulcerative colitis cannot be weaned off steroids without a relapse of the ulcerative colitis. Particularly steroid dependent ulcerative colitis includes ulcerative colitis wherein either:

(i) it is not possible to reduce the steroid dose below the equivalent of prednisolone 10 mg/day within three months from initiating steroid treatment without a relapse or flare of the ulcerative colitis; or
(ii) ulcerative colitis which relapses or flares within 3 months of stopping steroids.

The composition for the use of the invention may be for use in the treatment of thiopurine immunomodulator refractory ulcerative colitis. The ulcerative colitis may, for example be moderate or severe, thiopurine immunomodulator refractory ulcerative colitis.

“Thiopurine immunomodulator refractory colitis” refers to ulcerative colitis which is active or which relapses or flares in spite of being treated with a thiopurine. Particularly thiopurine immunomodulator refractory ulcerative colitis refers to ulcerative colitis which is active or which relapses or flares in spite of being treated with a thiopurine for at least 3 months, for example azathioprine 2-2.5 mg/kg/day or mercaptopurine 1-1.5 mg/kg/day.

When ulcerative colitis is in remission, a maintenance therapy may be required to keep the ulcerative colitis in remission. For example a maintenance therapy may prevent or reduce the risk of a flare or relapse of the ulcerative colitis. A maintenance therapy may also be used to reduce the frequency and/or severity of a flare or relapse of the ulcerative colitis.

The modified release composition may be used in accordance with the invention for use in a maintenance of remission treatment of ulcerative colitis. Accordingly, there is provided an oral modified release composition comprising cyclosporin, wherein the composition is for use in a maintenance of remission treatment of ulcerative colitis in a patient, wherein the composition is for use in the concurrent treatment of the patient with the cyclosporin and an active agent selected from an aminosalicylate and a steroid, and a fixed or free combination thereof. The active agent used concurrently with the composition in the maintenance treatment may be any of the active agents described herein.

Currently there are no curative drug treatments for ulcerative colitis. Therefore, a maintenance of remission treatment of ulcerative colitis may be required for long periods of time, of many weeks, months, years or possible for the life-time of the patient in order to maintain the ulcerative colitis in remission. Generally, long term steroid usage is undesirable due to the side effects associated with chronic steroid treatment. Use of the composition concurrently with an active agent in accordance with the present invention may enable the steroid dose required to maintain remission to be reduced or eliminated.

In a particular embodiment the composition is for use in a maintenance of remission treatment of ulcerative colitis concurrently with the agent, wherein the agent is an aminosalicylate. The composition may be administered to the patient over a prolonged period of time so as to maintain the ulcerative colitis in remission as described herein, for example for a year or longer, or any of the times described herein for maintenance of remission treatment of ulcerative colitis.

It is generally desirable to reduce or eliminate the use of steroids in the treatment of ulcerative colitis to reduce the undesirable side effects associated with steroid use. The use of the composition and the agent concurrently for the treatment of ulcerative colitis may enable the dose of steroid administered to a patient to be reduced or eliminated.

An aspect of the invention provides an oral modified release composition comprising cyclosporin, wherein the composition is for use in the treatment of ulcerative colitis in a patient, wherein the composition is for use in a dosage regimen wherein the patient is administered a first dosing regimen comprising the composition and (i) the aminosalicylate and the steroid or (ii) the steroid; and one or more subsequent dosage regimen comprising the composition and (i) the aminosalicylate and the steroid; or (ii) the steroid; and wherein

the total daily dose of the steroid in the first dosage regimen is greater than the total daily dose of the steroid in at least one of the subsequent dosage regimen.

The first dosage regimen uses an active agent which is or comprises a steroid. The first dosage regimen may therefore be used as an initial induction treatment to induce a remission or a response in the ulcerative colitis. The first dosage regimen may therefore be used for a sufficient time to provide remission, or a clinical response of the ulcerative colitis. The first dosage regimen may be used for a period of for example 4 weeks or more, 8 weeks or more, or 12 weeks or more, for example about 1 week to about 26 weeks, about 1 week to about 12 weeks; between about 1 and about 8 weeks; between about 1 week and about 4 weeks; about 1 week; about 2 weeks; about 3 weeks; about 4 weeks; about 5 weeks; about 6 weeks; about 7 weeks; about 8 weeks, about 12 week or about 26 weeks.

The daily steroid dose used in the subsequent dosage regimen is lower than the daily steroid dose used in the first dosage regimen and may be selected so as to maintain a clinical response or to maintain the patient in remission.

Optionally the daily steroid dosage used in the subsequent dosage regimen may be tapered over a period of time such that the daily dose of steroid is further reduced or eliminated from the subsequent dosage regimen.

Accordingly the subsequent dosage regimen may comprise two or more treatment cycles comprising the concurrent treatment of the patient with the composition, a steroid and optionally an aminosalicylate, wherein the daily dose of steroid administered to the patient is reduced after the completion of each treatment cycle. A treatment cycle may for example be of from about 1 day to about 12 weeks; about 1 week to about 8 weeks; about 1 week to about 6 weeks; or about 1 to about 4 weeks in duration, for example from about 1 to about 2 weeks in duration or suitably about 1 week in duration. Each treatment cycle may be of the same or a different duration. The daily steroid dose administered to the patient after the completion of each treatment cycle may be reduced by, for example by about 1 to about 10 mg or by about 1 to about 5 mg. By way of example a patient is treated with a daily steroid dose of 30 mg/day concurrently with the composition for a treatment cycle lasting 1 week. At the completion of the 1 week treatment cycle, the daily steroid dose is reduced by 5 mg and the patient is treated for a second treatment cycle lasting 1 week with a daily steroid dose of 25 mg concurrently with the composition. Further treatment cycles may be performed with a reduced steroid dose in each cycle optionally until the steroid dose is eliminated.

In a particular embodiment the daily steroid dosage used in the subsequent dosing regimen is reduced by for example about 5 mg every week until the daily steroid dose reaches, for example about 20 mg. Thereafter the daily steroid dose is reduced by for example about 2.5 mg every week, optionally until the steroid is eliminated.

Once the ulcerative colitis is in remission the composition (optionally with other drugs, for example an aminosalicylate) may be administered to the patient as a maintenance of remission treatment. The maintenance treatment may be used for prolonged periods of time as described herein. Suitable times for maintenance are any of those described herein, for example at least 1 year.

In a further embodiment there is provided an oral modified release composition comprising cyclosporin, wherein the composition is for use in the treatment of ulcerative colitis in a patient, wherein the composition is for use in a dosage regimen wherein the patient is administered a first dosing regimen comprising the composition and (i) the aminosalicylate and the steroid or (ii) the steroid; and one or more subsequent dosage regimen comprising the composition alone or the composition and an aminosalicylate, wherein the subsequent dosage regimen does not comprise a steroid.

Accordingly in any of the embodiments discussed above which comprise a first dosage regimen, the first dosing regimen may be used to provide an induction treatment of ulcerative colitis wherein the first dosage regimen provides remission of the ulcerative colitis and the subsequent dosage regimen provides a maintenance of remission treatment, suitably a steroid-free maintenance of remission treatment comprising administration of the composition alone or concurrently with an aminosalicylate. In one embodiment the maintenance of remission treatment comprises the oral administration of the modified release composition as a monotherapy. In a further embodiment the maintenance of remission treatment comprises the oral administration of the modified release composition concurrently with an aminosalicylate. Optionally in this embodiment, the dose of the aminosalicylate may be reduced or eliminated over a period of time as described herein so as to minimise the drug load administered to the patient whilst maintaining remission of the ulcerative colitis. As will be understood, reference anywhere herein to a “steroid-free maintenance of remission treatment” means that the patient is not treated concurrently with a steroid as part of the maintenance of remission treatment.

The embodiments above describing first and subsequent dosing regimens may be used in the treatment of any of the ulcerative colitis described herein for example moderate or severe ulcerative colitis which is optionally steroid dependent or steroid refractory ulcerative colitis.

As described herein the composition may be for use in an induction treatment of ulcerative colitis. Accordingly there is provided an oral modified release composition comprising cyclosporin, wherein the composition is for use in an induction treatment of ulcerative colitis in a patient, wherein the composition is for use in the concurrent treatment of the patient with the cyclosporin and an active agent selected from an aminosalicylate and a steroid, and a fixed or free combination thereof. The induction treatment may provide a remission or a clinical response of the ulcerative colitis.

When the composition is for use in an induction treatment of ulcerative colitis, the induction treatment may comprise two or more treatment cycles comprising the concurrent treatment of the patient with the composition, and the active agent(s). A treatment cycle may for example be of from about 1 day to about 12 weeks or more for example from 1 day to about 12 weeks; about 1 week to about 8 weeks; about 1 week to about 6 weeks; or about 1 to about 4 weeks in duration, for example from about 1 to about 2 weeks in duration or suitably about 1 week in duration. Each treatment cycle may be of the same or a different duration. The dose (for example the daily dose) of cyclosporin administered in each treatment cycle may be the same or different. The dose (for example the daily dose) of the active agent(s) administered in each treatment cycle may be the same or different. For example the patient may be treated in a first treatment cycle comprising a first dose of cyclosporin and a first dose of the active agent(s); and a second treatment cycle comprising a second dose of the cyclosporin and a second dose of the active agent(s); wherein the first dose of cyclosporin is different to the second dose of cyclosporin; and/or the first dose of active agent(s) is different to the second dose of active agent(s). The doses of the cyclosporin and/or active agent(s) used in the first treatment cycle may independently be the same or may be higher or lower than the corresponding doses used in the second treatment cycle. For example the first treatment cycle may comprise administration of a first dose of cyclosporin, a first dose of an aminosalicylate and optionally a first dose of a steroid; and the second treatment cycle may comprise a second dose of the cyclosporin, a second dose of the aminosalicylate and optionally a second dose of the steroid; and wherein any one or more of the following applies:

(i) the second dose of cyclosporin is the same, higher or lower than the first dose of cyclosporin; and/or
(ii) the second dose of steroid is the same, higher or lower than the first dose of steroid; and/or
(iii) the second dose of aminosalicylate is the same, higher or lower than the first dose of aminosalicylate;
(ii) the second dose of cyclosporin is lower than the first dose of cyclosporin.

Also contemplated is an induction treatment comprising a first treatment cycle and one or more subsequent treatment cycles; wherein the first treatment cycle comprises administering the composition comprising cyclosporin concurrently with the active agent(s); and wherein one or more of the dose of cyclosporin and/or an aminosalicylate and/or steroid in the subsequent treatment cycle is reduced or omitted. For example the cyclosporin may be omitted from the subsequent treatment cycle and the patient is treated with an aminosalicylate or a steroid, or a steroid and an aminosalicylate. Alternatively the aminosalicylate is omitted from the subsequent treatment cycle and the patient is treated with cyclosporin, a steroid or cyclosporin and a steroid.

When the composition if for use in any of the induction treatments of ulcerative colitis described herein, (e.g. to induce remission of ulcerative colitis) the active agent may optionally be or comprise a steroid in the form of an oral modified release formulation comprising the steroid. Such oral modified release formulations comprising a steroid are described in more detail below the section “steroids”. Accordingly in this embodiment the composition comprising cyclosporin is used concurrently with an active agent for the induction treatment of ulcerative colitis, wherein the active agent is or comprises a modified release formulation comprising a steroid. Optionally in this embodiment the active agent may further comprise an aminosalicylate. When used, the aminosalicylate may be used as a fixed combination with the steroid or be administered separately (for example to provide simultaneous, separate or sequential administration of the steroid and the aminosalicylate). In a further embodiment modified release composition comprising cyclosporin may further comprise the steroid, such that the composition provides modified release of the cyclosporin and the steroid, for example release of the cyclosporin and the steroid in at least the colon.

As mentioned above it is desirable to provide a maintenance of remission treatment of ulcerative colitis which does not require the use of steroids. A suitable maintenance of remission treatment for ulcerative colitis may be provided by the use of an oral modified release composition comprising cyclosporin.

Accordingly, there is provided an oral modified release composition comprising cyclosporin, wherein the composition is for use in a maintenance of remission treatment of ulcerative colitis in a patient wherein the ulcerative colitis is in remission. The composition is this aspect of the invention is used to maintain the ulcerative colitis in remission. Suitably in this embodiment the patient is not treated for ulcerative colitis with any steroid treatment, accordingly the maintenance of remission treatment is a steroid-free maintenance of remission treatment. The ulcerative colitis is therefore maintained in steroid-free remission. The modified release composition used in this aspect of the invention may be any of the modified release compositions comprising cyclosporin described herein. The maintenance of remission treatment may be used for prolonged periods of time, for example at least a year or any of the time periods mentioned herein in relation to the maintenance treatments described herein.

Certain patients with ulcerative colitis may not respond or may be intolerant to conventional treatments for ulcerative colitis. For example the patient may be intolerant to a particular treatment for ulcerative colitis because of side effects associated with the treatment. Alternatively as described herein a patient may be non-responsive, for example may be refractory to a treatment as hereinbefore described. A modified release composition comprising cyclosporin, wherein the composition is for use alone in the treatment of ulcerative colitis (particularly moderate or severe ulcerative colitis) in such non-responsive or intolerant patients may therefore be beneficial.

A further aspect of the invention provides an oral modified release composition comprising cyclosporin, wherein the composition is for use in administration alone, or concurrently with an active agent selected from an aminosalicylate, a steroid and a free or fixed combination thereof, the composition being for use in the treatment of:

(a) moderate or severe active ulcerative colitis in a patient, wherein the patient is non-responsive or intolerant to prior treatment with one or more of an aminosalicylate a steroid, azathioprine or 6-mercaptopurine (6-MP); or

(b) moderate or severe active steroid dependent ulcerative colitis in a patient.

In this embodiment, suitably the patient has not been treated with a biological ulcerative colitis treatment, for example a TNF-antibody therapy or an integrin inhibitor antibody such as vedolizumab prior to treatment with the composition alone or the composition concurrently with the said active agent. In one aspect within this embodiment the composition is for use alone in the treatment of the patient (i.e. without concurrent use of the active agent). In another aspect the composition is for use concurrently with the active agent.

The Examples herein show that patients with moderate ulcerative colitis treated with a modified release composition comprising cyclosporin showed beneficial effects in for example, clinical response or remission of the ulcerative colitis.

A further aspect of the invention provides an oral modified release composition comprising cyclosporin, wherein the composition is for use in the treatment of moderate ulcerative colitis in a patient. Accordingly in this embodiment of the invention a patient with moderate ulcerative colitis may be treated with the oral modified release composition alone. The ulcerative colitis may affect some or all of the colon as described in any of the embodiments herein. The modified release composition for use in this aspect of the invention may be any of the modified release compositions comprising cyclosporin described herein. The composition in this embodiment may, for example, be for use in an induction treatment of moderate ulcerative colitis. When used in an induction treatment of moderate ulcerative colitis, used of the composition may induce remission or a clinical response of the moderate ulcerative colitis.

It is to be understood that methods of treatment corresponding to any of the uses of the oral modified release composition in the treatment of ulcerative colitis described herein are intended to be encompassed within the invention. Similarly, any of the uses described herein may be described in relation to the use of the composition in the manufacture of a medicament for use in any of the treatments of ulcerative colitis described herein. The invention encompasses all such corresponding uses in the manufacture of a medicament.

### Active Agent

The composition comprising cyclosporin is used concurrently with an active agent selected from an aminosalicylate and a steroid and a fixed or free combination thereof. The patient may be treated concurrently with the composition and an aminosalicylate alone. The patient may be treated concurrently with the composition and a steroid alone. The patient may be treated with composition, an aminosalicylate and a steroid. The invention contemplates the use of two or more aminosalicylates and or two or more steroids. When the active agent comprises an aminosalicylate and a steroid they may be administered to the patient together as a fixed combination. Alternatively they may be administered to the patient separately. The aminosalicylate and steroid may be administered to the patient by the same or different routes of administration.

The active agent and composition may be used concurrently with other treatments for ulcerative colitis as described herein. However, in particular aspects of the invention the ulcerative colitis is treated exclusively with the composition and with one or both of an aminosalicylate and a steroid. Accordingly in this embodiment the patient is not treated with for example, a thiopurine immunomodulator such as azathioprine or 6-mercaptopurine; an anti-TN F antibody therapy for example infliximab, adalimumab, or golimumab; an integrin inhibitor antibody, for example vedolizumab; tacrolimus or cyclosporin (other than the cyclosporin in the composition, for example the patient is not treated with intravenous cyclosporin, or an instant release composition comprising cyclosporin for example Neoral™ or Sandimmune™). In further particular embodiments, prior to the administration of the modified release composition comprising cyclosporin, the patient has not been treated with any one or more of a thiopurine immunomodulator such as azathioprine or 6-mercaptopurine; a biological treatment of ulcerative colitis, for example an anti-TNF antibody (optionally infliximab, adalimumab, or golimumab) or an integrin inhibitor antibody (for example vedolizumab); tacrolimus or cyclosporin. In other words the patient is treatment naïve to at least one of these therapies prior to the administration of the modified release composition comprising cyclosporin.

### Aminosalicylate

As used herein, “aminosalicylate” refers to 4- or 5-amino-2-hydroxybenzoic acid or a pro drug or a pharmaceutically acceptable salt thereof. Particularly the aminosalicylate is 5-amino-2-hydroxybenzoic acid or a pro drug or a pharmaceutically acceptable salt thereof. Suitable pro drugs are compounds which produce 4- or 5-amino-2-hydroxybenzoic acid in-vivo following administration of the compound. Examples of pro drugs include azo derivatives of 2-hydroxybenzoic acid for example sulfasalazine (2-hydroxy-5-[[4-[(2-pyridinylamino)sulfonyl]phenyl]azo]-benzoic acid); olsalazine (3,3′-dicarboxy-4,4′-dihydroxyazobenzene) and balsalazide ((E)-5-[[4-[[(2-carboxyethyl)amino]carbonyl]phenyl]azo]-2-hydroxybenzoic acid); ipsalazide (5-[[4-[(carboxmethyl)carbamoyl]phenyl]azo]-2-hydroxybenzoic acid); or benzalazine ((2-hydroxy-5-[(4-carboxyphenyl)azo]benzoic acid); or a pharmaceutically acceptable salt of any of the foregoing.

5-amino-2-hydroxybenzoic acid is also known as “mesalamine”, “mesalazine” and “5-ASA”

The aminosalicylate may be selected from the group consisting of mesalazine, sulfasalazine, olsalazine, ipsalazide, balsalazide and benzalazine, or a pharmaceutically acceptable salt thereof. Particularly the aminosalicylate is mesalazine (5-ASA) or a pharmaceutically acceptable salt thereof.

The aminosalicylate may, for example, be administered to the patient orally and/or rectally. Optionally the aminosalicylate is orally administered. Optionally the aminosalicylate is rectally administered. Optionally the aminosalicylate is rectally and orally administered. Rectal administration may be particularly suitable when the ulcerative colitis is ulcerative proctosigmoiditis or left-sided ulcerative colitis.

The aminosalicylate may, for example, be administered to the patient in the form of an instant release formulation or as a modified release formulation. For example the aminosalicylate may be administered as a modified release composition which releases the aminosalicylate in the colon. Oral compositions comprising an aminosalicylate are well-known, for example, Asacol™, Asacol™ HD, Apriso™, Pentasa™, Lialda™ Mezavant™, Mezavant™ XL, Salofalk™, Dipentum™, Colazal™, Giazo™ or Azulfidine™.

Compositions comprising an aminosalicylate suitable for rectal administration are well known, for example Rowasa™ or Canasa™.

The dosage of aminosalicylate will depend upon the severity and the extent of the ulcerative colitis. Suitable dosages are well known to physicians and will typically be a total daily dose in the range of about 500 mg to about 10 g, suitably from about 1 g to about 8 g, from about 1 g to about 5 g, about 2 g to about 5 g or about 2 g to about 3 g. Optionally a patient with moderate ulcerative colitis may be dosed with about 4 to about 5 g per day of aminosalicylate as an induction dose. Subsequent dosages may then be reduced if the patient responds to the treatment. For example the subsequent dosage may be reduced, for example to provide a maintenance treatment dose of from about 500 mg to about 3 g per day, from about 500 mg to about 2 g per day or about 2 g to about 3 g per day. The aminosalicylate may be taken once or twice per day.

When the active agent is an aminosalicylate the ulcerative colitis may for example be mild or moderate ulcerative colitis.

### Steroid

The active agent may be or comprise a steroid. The steroid may be any steroid produced by the adrenal cortex, including glucocorticoids and mineralocorticoids, and synthetic analogues and derivatives of naturally occurring corticosteroids having anti-inflammatory activity. Particularly the steroid is a glucocorticosteroid. Examples of corticosteroids that can be used are aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids mentioned in this paragraph.

The steroid may be administered to the patient by any suitable route, for example orally, rectally, intravenously or a combination of two or more such routes. For example the steroid may be administered orally and rectally. Steroids suitable for oral administration include cortisone, prednisone, prednisolone or budesonide. Steroids suitable for rectal administration include, for example beclomethasone, tixicortol, budesonide or prednisolone. Steroids suitable for intravenous administration include, for example prednisolone, methylprednisolone or corticotropin.

The dose of steroid administered to the patient will vary depending upon the particular steroid, the route of administration and the severity of the ulcerative colitis. Suitable dosages of steroids are well known to physicians. Optionally a steroid such as prednisolone, may be orally administered at a dose of from about 1 mg to about 50 mg per day; from about 10 to about 50 mg per day or from about 15 mg to about 45 mg per day. Generally the steroid is administered once per day.

Suitably as discussed herein the dose of oral steroid may be reduced or tapered as the patient responds to the steroid treatment, optionally until the oral steroid treatment can be stopped. Generally the reduction or tapering is gradual and the daily dose of steroid is reduced on a weekly basis.

In one aspect of the invention the active agent is or comprises an oral modified release formulation comprising the steroid. Suitably the modified release formulation releases the steroid in the lower GI tract, for example in the colon. Suitably the steroid in the modified release formulation is a steroid with low systemic bioavailability, for example budesonide or beclomethasone dipropionate. Release of the steroid locally in the colon may reduce the systemic side effects associated with the steroid.

Representative doses of intravenously administered steroids include hydrocortisone at about 300 to about 400 mg/day or methylprednisolone a about 40 to about 60 mg/day. Intravenous steroids are generally used for five to seven days.

Modified release formulations comprising a steroid (for example budesonide) are known, as described in, for example WO00/76478. Modified release formulations are well known, for example Uceris™/Cortiment™, a modified release formulation comprising budesonide.

Accordingly the steroid in the form of an oral modified release formulation comprising a steroid may be used concurrently with the oral modified release composition comprising cyclosporin for use in any of the treatments of ulcerative colitis described herein. Particularly a modified release formulation comprising a steroid may be used concurrently with the modified release composition comprising cyclosporin in accordance with the invention for use in inducing remission of ulcerative colitis, more particularly for use in inducing remission of mild or moderate ulcerative colitis.

In a further aspect the oral modified release composition comprising cyclosporin may further comprise a steroid. A modified release composition comprising cyclosporin and a steroid for use in the treatment of ulcerative colitis would provide release (for example into the colon) of the cyclosporin and the steroid from the same composition. Such a composition would therefore conveniently provide a dose of both cyclosporin and steroid by oral administration of a single modified release composition. Such a combination dosage form may be particularly useful for use in inducing remission of ulcerative colitis, optionally for use in inducing remission of mild or moderate ulcerative colitis, or optionally for inducing remission in moderate or severe ulcerative colitis.

### Cyclosporin

The modified release composition comprises cyclosporin. Cyclosporines form a class of polypeptides commonly possessing immunosuppressive and anti-inflammatory activity. The most commonly known cyclosporin is cyclosporin-A. Other forms of cyclosporines include cyclosporin-B, -C, -D, and -G and their derivatives. It should be understood that herein the terms “cyclosporin” or “cyclosporins” refers to any of the several cyclosporins, derivatives or prodrugs thereof, or to any mixture of any of the above. Suitably the cyclosporin is cyclosporin A.

### Oral Modified Release Composition Comprising Cyclosporin

The modified release composition may comprise a matrix and cyclosporin. The matrix may be formed with a hydrogel-forming polymer, and may contain additional excipient(s) to the polymer. The cyclosporin is contained within the matrix. The cyclosporin may be in solution or in suspension, or in a combination thereof; however the invention is not limited to formulations comprising a solution or suspension of the cyclosporin and it includes, for example, cyclosporin encapsulated in liposomes or cyclodextrin. The matrix may contain inclusions in which the cyclosporin is comprised; for example, the inclusions may comprise a hydrophobic medium in which the cyclosporin is dissolved or suspended. Cyclosporin may therefore be directly dissolved or suspended in the matrix, or it may be dissolved or suspended indirectly in the matrix by way of inclusions in which the active ingredient is dissolved or suspended.

The composition may therefore comprise a matrix-forming polymer, in particular a hydrogel-forming polymer. The matrix of the composition may be or comprise a polymer matrix comprising a polymer selected from a water-permeable polymer, a water-swellable polymer and a biodegradable polymer. In particular, the matrix is or comprises a hydrogel-forming polymer described in more detail below.

The matrix material may be or may comprise water-soluble polymer, an oligosaccharide and/or a wax The matrix material may comprise or be a hydrophobic polymer (for example selected from poly(amides), poly(amino-acids), hyaluronic acid; lipo proteins; poly(esters), poly(orthoesters), poly(urethanes) or poly(acrylamides), poly(glycolic acid), poly(lactic acid) and corresponding co-polymers (poly(lactide-co-glycolide acid); PLGA); siloxane, polysiloxane; dimethylsiloxane/-methylvinylsiloxane copolymer; poly(dimethylsiloxane/-methylvinylsiloxane/-methylhydrogensiloxane) dimethylvinyl or trimethyl copolymer; silicone polymers e.g. siloxane; alkyl silicone; silica, aluminium silicate, calcium silicate, aluminium magnesium silicate, magnesium silicate, diatomaceous silica, or a combination thereof).

Modified release of the cyclosporin from the composition may be achieved by virtue of the properties of the matrix material. For example the matrix may be a permeable or erodible polymer within which the cyclosporin is contained, e.g. dissolved or dispersed; following oral administration the matrix is gradually dissolved or eroded thereby releasing cyclosporin from the matrix. Erosion may also be achieved by biodegradation of a biodegradable polymer matrix. Where the matrix is permeable, water permeates the matrix enabling the drug to diffuse from the matrix. Polymeric modified release matrix materials include cellulose derivatives, for example hydroxypropylmethyl cellulose, poly(lactic acid), poly(glycoloic)acid, poly(lactic-co glycolic acid copolymers), polyethylene glycol block co-polymers, polyorthoesters, polyanhydrides, polyanhydride esters, polyanhydride imides, polyamides and polyphosphazines. A matrix formed with a hydrogel-forming polymer may therefore include one or more such modified release polymer(s).

### Modified Release Coatings

In preferred embodiments of the invention modified release of cyclosporin is achieved wholly or in part through the use of one or more suitable coatings on a core containing a cyclosporin. The term “modified release” is intended to encompass controlled release, extended (or sustained) release and delayed release or any combination thereof, for example delayed and controlled release of cyclosporin from the composition following oral administration of the composition. Reference to a coating “to control or modulate release” used herein therefore includes the modified release coatings described in this section and elsewhere.

Thus according to one embodiment of the present invention, there is provided a modified release composition comprising a core, wherein the core comprises cyclosporin, and the core bears a modified release coating outside the core (i.e. is coated) in order to modulate release of cyclosporin from the core.

The modified release coating may be present in an amount described elsewhere in this specification.

The core is preferably in the form of a minibead as described hereafter in more detail. The modified release coating may be a film or it may be a membrane. The modified release coating, e.g. film or membrane, may serve to delay release until after the stomach; the coat may therefore be an enteric coat. The coat may comprise one or more substances preferably of a polymeric nature (e.g. methacrylates etc; polysaccharides etc as described in more detail below) or combination of more than one such substance, optionally including other excipients, for example, plasticizers. Preferred plasticizers, if they are used, include hydrophilic plasticizers for example triethyl citrate (TEC) which is particularly preferred when using the Eudragit™ family of polymers as coatings as described below. Another preferred plasticiser, described in more detail below in relation to coating with ethyl cellulose, is dibutyl sebacate (DBS). Alternative or additional optionally included excipients are glidants. A glidant is a substance that is added to a powder or other medium to improve its flowability. A typical glidant is talc which is preferred when using the Eudragit™ family of polymers as coatings. The modified release coating may comprise two or more of the polymeric modified release coatings described herein to tailor the release of cyclosporin from the composition to the desired region of the GI tract, preferably the colon, following oral administration of the composition.

The modified release coating may be applied as described below and may vary as to thickness and density. The amount of modified release coating is defined by the additional weight added to (gained by) the dry composition (e.g. bead) to which it is applied. Weight gain due to the modified release coating is suitably in the range 0.1% to 50%, for example 5% to 40% or from 1% to 18%; or from 1 to 15% of the dry weight of the bead, more preferably in the range 3% to 10% or in the range 5-12% or in the range 7-12%.

The thickness of the modified release coating may be from 1 μm to 1 mm, but is suitably 1 μm to 150 μm, for example for 1 to 100 μm. Suitably the modified release coating provides a coating thickness on the composition of from about 10 μm to about 1 mm, for example, from about 10 μm to about 500 μm, from about 50 μm to about 1 mm, or about from about 50 μm to about 500 μm. The thickness may therefore be from about 100 μm to about 1 mm, e.g. 100 μm to about 750 μm or about 100 μm to about 500 μm. The thickness may be from about 250 μm to about 1 mm, e.g. about 250 μm to about 750 μm or 250 μm to about 500 μm. The thickness may be from about 500 μm to about 1 mm, e.g. about 750 μm to about 1 mm or about 500 μm to about 750 μm. The thickness may therefore be from about 10 μm to about 100 μm, e.g. from about 10 μm to about 50 μm or about 50 μm to about 100 μm.

The polymeric coating material of the modified release coating may comprise methacrylic acid co-polymers, ammonio methacrylate co-polymers, or mixtures thereof. Methacrylic acid co-polymers such as, for example, EUDRAGIT™ S and EUDRAGIT™ L (Evonik) are particularly suitable. These polymers are gastroresistant and enterosoluble polymers. Their polymer films are insoluble in pure water and diluted acids. They may dissolve at higher pHs, depending on their content of carboxylic acid. EUDRAGIT™ S and EUDRAGIT™ L can be used as single components in the polymer coating or in combination in any ratio. By using a combination of the polymers, the polymeric material can exhibit solubility at a variety of pH levels, e.g. between the pHs at which EUDRAGIT™ L and EUDRAGIT™ S are separately soluble. In particular, the coating may be an enteric coating comprising one or more co-polymers described in this paragraph. A particular coating material to be mentioned is Eudragit L 30 D-55.

The trademark “EUDRAGIT” is used hereinafter to refer to methacrylic acid copolymers, in particular those sold under the EUDRAGIT™ by Evonik.

The modified release coating can comprise a polymeric material comprising a major proportion (e.g., greater than 50% of the total polymeric coating content) of at least one pharmaceutically acceptable water-soluble polymer, and optionally a minor proportion (e.g., less than 50% of the total polymeric content) of at least one pharmaceutically acceptable water insoluble polymer. Alternatively, the membrane coating can comprise a polymeric material comprising a major proportion (e.g., greater than 50% of the total polymeric content) of at least one pharmaceutically acceptable water insoluble polymer, and optionally a minor proportion (e.g., less than 50% of the total polymeric content) of at least one pharmaceutically acceptable water-soluble polymer.

Ammonio methacrylate co-polymers such as, for example, EUDRAGIT™ RS and EUDRAGIT™ RL (Evonik) are suitable for use in the present invention. These polymers are insoluble in pure water, dilute acids, buffer solutions, and/or digestive fluids over the entire physiological pH range. The polymers swell in water and digestive fluids independently of pH. In the swollen state, they are then permeable to water and dissolved active agents. The permeability of the polymers depends on the ratio of ethylacrylate (EA), methyl methacrylate (MMA), and trimethylammonioethyl methacrylate chloride (TAMCl) groups in the polymer. For example, those polymers having EA:MMA:TAMCl ratios of 1:2:0.2 (EUDRAGIT™ RL) are more permeable than those with ratios of 1:2:0.1 (EUDRAGIT™ RS). Polymers of EUDRAGIT™ RL are insoluble polymers of high permeability. Polymers of EUDRAGIT™ RS are insoluble films of low permeability. A diffusion-controlled pH-independent polymer in this family is RS 30 D which is a copolymer of ethyl acrylate, methyl methacrylate and a low content of methacrylic acid ester with quaternary ammonium groups present as salts to make the polymer permeable. RS 30 D is available as an aqueous dispersion.

The amino methacrylate co-polymers can be combined in any desired ratio, and the ratio can be modified to modify the rate of drug release. For example, a ratio of EUDRAGIT™ RS:EUDRAGIT™ RL of 90:10 can be used. Alternatively, the ratio of EUDRAGIT™ RS:EUDRAGIT™ RL can be about 100:0 to about 80:20, or about 100:0 to about 90:10, or any ratio in between. In such formulations, the less permeable polymer EUDRAGIT™ RS generally comprises the majority of the polymeric material with the more soluble RL, when it dissolves, permitting gaps to be formed through which solutes can come into contact with the core allowing for the active to escape in a controlled manner.

The amino methacrylate co-polymers can be combined with the methacrylic acid co-polymers within the polymeric material in order to achieve the desired delay in the release of the drug and/or poration of the coating and/or exposure of the composition within the coating to allow egress of drug and/or dissolution of the immobilization or water-soluble polymer matrix. Ratios of ammonio methacrylate co-polymer (e.g., EUDRAGIT™ RS) to methacrylic acid co-polymer in the range of about 99:1 to about 20:80 can be used. The two types of polymers can also be combined into the same polymeric material, or provided as separate coats that are applied to the beads.

Eudragit™ FS 30 D is an anionic aqueous-based acrylic polymeric dispersion consisting of methacrylic acid, methyl acrylate, and methyl methacrylate and is pH sensitive. This polymer contains fewer carboxyl groups and thus dissolves at a higher pH (>6.5). The advantage of such a system is that it can be easily manufactured on a large scale in a reasonable processing time using conventional powder layering and fluidized bed coating techniques. A further example is EUDRAGIT™ L 30D-55 which is an aqueous dispersion of anionic polymers with methacrylic acid as a functional group. It is available as a 30% aqueous dispersion.

In addition to the EUDRAGIT™ polymers described above, a number of other such copolymers can be used to control drug release. These include methacrylate ester co-polymers such as, for example, the EUDRAGIT™ NE and EUDRAGIT™ NM ranges. Further information on the EUDRAGIT™ polymers can be found in “Chemistry and Application Properties of Polymethacrylate Coating Systems,” in Aqueous Polymeric Coatings for Pharmaceutical Dosage Forms, ed. James McGinity, Marcel Dekker Inc., New York, pg 109-114 the entirety of which is incorporated herein by reference.

Several derivatives of hydroxypropyl methylcellulose (HPMC) also exhibit pH dependent solubility and may be used in the invention for the modified release coating. As examples of such derivatives may be mentioned HPMC esters, for example hydroxypropyl methylcellulose phthalate (HPMCP), which rapidly dissolves in the upper intestinal tract and hydroxypropyl methylcellulose acetate succinate (HPMCAS) in which the presence of ionisable carboxyl groups causes the polymer to solubilize at high pH (>5.5 for the LF grade and >6.8 for the HF grade). These polymers are commercially available from Shin-Etsu Chemical Co. Ltd. As with other polymers described herein as useful for delayed release coatings, HPMC and derivatives (e.g. esters) may be combined with other polymers e.g. EUDRAGIT RL-30 D.

Other polymers may be used to provide a modified release coating in particular enteric, or pH-dependent, polymers. Such polymers can include phthalate, butyrate, succinate, and/or mellitate groups. Such polymers include, but are not limited to, cellulose acetate phthalate, cellulose acetate succinate, cellulose hydrogen phthalate, cellulose acetate trimellitate, hydroxypropyl-methylcellulose phthalate, hydroxypropylmethyl cellulose acetate succinate, starch acetate phthalate, amylose acetate phthalate, polyvinyl acetate phthalate, and polyvinyl butyrate phthalate.

### pH Independent Polymer Modified Release Coatings

In a particular embodiment the modified release coating is or comprises a polymeric coating which is pH-independent in its dissolution profile and/or in its ability to release the cyclosporin incorporated in the compositions of the invention. A pH-independent polymer modified release coating comprises a modified release polymer, optionally a plurality of modified release polymers, and one or more other optional components. The other components may serve to modulate the properties of the formulation Examples have already been given (e.g., Eudragit RS and RL).

Another example of a pH-independent polymeric modified release coating is a coating comprising ethylcellulose. It will be understood that an ethylcellulose composition for use in coating a dosage form may comprise, in addition to ethylcellulose and—in the case of a liquid composition—a liquid vehicle, one or more other components. The other components may serve to modulate the properties of the composition, e.g. stability or the physical properties of the coating such as the flexibility of the film coating. The ethylcellulose may be the sole controlled release polymer in such a composition. The ethylcellulose may be in an amount of at least 50%, at least 60%, at least 70%, at least 80%, at least 90% or at least 95% by weight of the dry weight of a coating composition for use in coating a dosage form. Accordingly, an ethylcellulose coating may include other components in addition to the ethylcellulose. The ethylcellulose may be in an amount of at least 50%, at least 60%, at least 70%, at least 80%, at least 90% or at least 95% by weight of the ethylcellulose coating. Suitably the ethyl cellulose coating further comprises a plasticizer as described below to improve the flexibility of the film and to improve the film-forming properties of the coating composition during application of the coating.

A particular ethylcellulose coating composition which may be applied to the compositions of the invention is a dispersion of ethylcellulose in a sub-micron to micron particle size range, e.g. from about 0.1 to 10 microns in size, homogeneously suspended in water with the aid of an emulsification agent, e.g. ammonium oleate. The ethylcellulose dispersion may optionally and preferably contain a plasticizer. Suitably plasticisers include for example dibutyl sebacate (DBS), diethylphthalate, triethyl citrate, tributyl citrate, triacetin, or medium chain triglycerides. The amount of plasticizer present in the coating composition will vary depending upon the desired properties coating. Typically the plasticizer comprises from 1 to 50%, for example about 8 to about 50% of the combined weight of the plasticizer and ethyl cellulose. Such ethylcellulose dispersions may, for example, be manufactured according to U.S. Pat. No. 4,502,888, which is incorporated herein by reference. One such ethylcellulose dispersion suitable for use in the present invention and available commercially is marketed under the trademark Surelease™, by Colorcon of West Point, Pa. USA. In this marketed product, the ethylcellulose particles are, e.g., blended with oleic acid and a plasticizer, then optionally extruded and melted. The molten plasticized ethylcellulose is then directly emulsified, for example in ammoniated water optionally in a high shear mixing device, e.g. under pressure. Ammonium oleate can be formed in situ, for instance to stabilize and form the dispersion of plasticized ethylcellulose particles. Additional purified water can then be added to achieve the final solids content. See also U.S. Pat. No. 4,123,403, which is incorporated herein by reference.

The trademark “Surelease™” is used hereinafter to refer to ethylcellulose coating materials, for example a dispersion of ethylcellulose in a sub-micron to micron particle size range, e.g. from about 0.1 to 10 microns in size, homogeneously suspended in water with the aid of an emulsification agent, e.g. ammonium oleate. In particular, the trademark “Surelease™” is used herein to refer to the product marketed by Colorcon under the Surelease™ trademark.

Surelease™ dispersion is an example of a combination of film-forming polymer, plasticizer and stabilizers which may be used as a coating to adjust rates of active principle release with reproducible profiles that are relatively insensitive to pH. The principal means of drug release is by diffusion through the Surelease™ dispersion membrane and is directly controlled by film thickness. Use of Surelease™ is particularly preferred and it is possible to increase or decrease the quantity of Surelease™ applied as coating in order to modify the dissolution of the coated composition. Unless otherwise stipulated, use of the term “Surelease” may apply to Surelease E-7-19020, E-7-19030, E-7-19040 or E-7-19050. An ethylcellulose coating formulation, for example Surelease E-7-19020, may comprise ethylcellulose blended with oleic acid and dibutyl sebacate, then extruded and melted. The molten plasticized ethylcellulose is then directly emulsified in ammoniated water in a high shear mixing device under pressure. Ammonium oleate is formed in situ to stabilize and form the dispersion of plasticized ethylcellulose particles. Additional purified water is then added to achieve the final solids content. An ethyl cellulose coating formulation, for example E-7-19030 additionally comprises colloidal anhydrous silica dispersed into the material. An ethyl cellulose coating formulation, for example E-7-19040, may comprise medium chain triglycerides instead of dibutyl sebacate, in particular, in particular in a formulation comprising colloidal anhydrous silica and oleic acid. An ethylcellulose coating formulation, for example Surelease E-7-19050, may derive from blending ethylcellulose with oleic acid before melting and extrusion. The molten plasticized ethylcellulose is then directly emulsified in ammoniated water in a high shear mixing device under pressure. Ammonium oleate is formed in situ to stabilize and form the dispersion of plasticized ethylcellulose particles. However, formulations that comprise medium chain triglycerides, colloidal anhydrous silica and oleic acid are preferred. Surelease E-7-19040 is particularly preferred.

The invention also contemplates using combinations of ethylcellulose, e.g. a Surelease formulation with other coating components, for example sodium alginate, e.g. sodium alginate available under the trade name Nutrateric™.

In addition to the EUDRAGIT™ and Surelease™ polymers discussed above, where compatible, any combination of coating polymers disclosed herein may be blended to provide additional controlled- or targeted-release profiles.

The delayed release coating can further comprise at least one soluble excipient to increase the permeability of the polymeric material. These soluble excipients can also be referred to or are pore formers. Suitably, the at least one soluble excipient or pore former is selected from among a soluble polymer, a surfactant, an alkali metal salt, an organic acid, a sugar, and a sugar alcohol. Such soluble excipients include, but are not limited to, polyvinyl pyrrolidone, polyvinyl alcohol (PVA), polyethylene glycol, a water-soluble hydroxypropyl methyl cellulose, sodium chloride, surfactants such as, for example, sodium lauryl sulfate and polysorbates, organic acids such as, for example, acetic acid, adipic acid, citric acid, fumaric acid, glutaric acid, malic acid, succinic acid, and tartaric acid, sugars such as, for example, dextrose, fructose, glucose, lactose, and sucrose, sugar alcohols such as, for example, lactitol, maltitol, mannitol, sorbitol, and xylitol, xanthan gum, dextrins, and maltodextrins; and a polysaccharide susceptible of degradation by a bacterial enzyme normally found in the colon, for example polysaccharides include chondroitin sulphate, pectin, dextran, guar gum and amylase, chitosan etc. and derivatives of any of the foregoing. In some embodiments, polyvinyl pyrrolidone, mannitol, and/or polyethylene glycol can be used as soluble excipients. The at least one soluble excipient can be used in an amount ranging from about 0.1% to about 15% by weight, based on the total dry weight of the polymer coating, for example from about 0.5% to about 10%, about 0.5% to about 5%, about 1% to about 3%, suitably about 2% based on the total dry weight of the polymer coating. The modified release coating may be free from HPMC.

The modifications in the rates of release, such as to create a delay or extension in release, can be achieved in any number of ways. Mechanisms can be dependent or independent of local pH in the intestine, and can also rely on local enzymatic activity to achieve the desired effect. Examples of modified-release formulations are known in the art and are described, for example, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566 all of which are incorporated herein by reference in their entirety.

The addition to Surelease™ or other pH-independent polymer substance of a second polymer (e.g. a polysaccharide, especially a heteropolysaccharide) which is susceptible to degradation by colonic bacterial enzymes (and optionally or alternatively by pancreatic or other relevant enzymes), helps to provide targeted release of cyclosporin to a site or sites within the GI tract where the second polymer is degraded. By varying the amount of second polymer added present in the coating the dissolution profile may be optimized to provide the required release of cyclosporin from the composition.

In a particular embodiments the modified release coating provides for release of the cyclosporin in at least the colon. Accordingly in one embodiment the coating comprises a combination of ethylcellulose (preferably a described above, and particularly formulated with an emulsification agent such as, for example, ammonium oleate and/or a plasticizer such as, for example, dibutyl sebacate or medium chain triglycerides) and a polysaccharide susceptible of degradation by a bacterial enzyme normally found in the colon. Such polysaccharides include chondroitin sulfate, pectin, dextran, guar gum and amylase, chitosan etc. and derivatives of any of the foregoing. Chitosan may be used in connection with obtaining a colon-specific release profile; additionally or alternatively, pectin may also be so used.

The use of polysaccharides by themselves for coating purposes has been tried with limited success. Most of the non-starch polysaccharides suffer from the drawback of lacking good film forming properties. Also, they tend to swell in the GI tract and become porous, resulting in the early release of the drug. Even amorphous amylose, which is resistant to degradation by pancreatic alpha amylase but capable of degradation by colonic bacterial enzymes has the disadvantage of swelling in aqueous media although this can be controlled by incorporating insoluble polymers, for example ethyl cellulose and/or acrylates into the amylose film. Amylose however is not water-soluble and although water-insoluble polysaccharides are not excluded, water-soluble polysaccharide (WSP) susceptible of bacterial enzymatic degradation brings particularly advantageous results when used as a coating in accordance with this embodiment of the present invention. A particularly preferred polysaccharide in this embodiment of the present invention is pectin. Various kinds of pectin may be used including pectin of different grades available i.e. with differing degrees of methylation (DM), i.e. percentage of carbonyl groups esterified with methanol, for example pectins with a DM of more than 50%, known as High Methoxy (HM) pectins or Low Methoxy (LM) pectins, or a pectin combination comprising an HM pectin and an LM pectin. It is also possible in this embodiment to use pectins having various degrees of acetylation (DAc). Taken together, the DM and DAc or the degree of substitution is known as Degree of Esterification (DE). Pectins of various DE's may be used according to the invention. As an alternative to pectin, sodium alginate may be used as a polysaccharide according to an embodiment of the invention. However, other embodiments may conveniently include amylose and/or starch which contains amylose. Various grades of starch, containing different percentages of amylose may be used including for example Hylon V (National Starch Food Innovation) which has an amylose percentage of 56% or Hylon VII which has an amylose percentage of 70%. The remaining percentage is amylopectin. The polysaccharides pectin, amylose and sodium alginate are particularly preferred for achieving colon delivery of the cyclosporin.

It has been found that water-soluble polysaccharide, suitably pectin, can act as a former of pores in the coating otherwise provided by ethylcellulose (preferably Surelease). By “pores” is not meant shaft-like holes from the surface to the core of the composition, rather areas of weakness or absence of coating occurring stochastically on and within the coating of the invention.

Pore formers have been described before in connection with Surelease™ (see e.g. US 2005/0220878). As mentioned above, pore formation may also be achieved by inclusion other soluble excipients within the polymer coating to increase the permeability of the polymeric material

According to a particular embodiment of the invention the modified release coating comprises ethyl cellulose, e.g. Surelease™ and a water-soluble polysaccharide (WSP) wherein the proportion of Surelease™ to WSP is ideally in the range 90:10 to 99:1, preferably, 95:5 to 99:1, more preferably 97:3 to 99:1, for example about 98:2 based upon the dry weight of the coating. Suitably in this and other embodiments described herein in which Surelease™ is used as a coating, the weight gain of the composition due to application of the coating comprising ethyl cellulose, (e.g. Surelease™ and WSP) is in the range of from 1 to 30% (for example from: 3% to 25%; 5% to 15%; 8% to 14%; 10% to 12%; 12% to 18%; or 16% to 18%, suitably the weight gain is about 11%, about 11.5%, or about 17%). It is particularly preferred that when a WSP is used in the coating the WSP is pectin. Particularly favoured weight gains using coatings comprising ethyl cellulose e.g. Surelease™ are those in the range 5-12%, 8-12% or 5 to 10%; suitably about 8%, about 8.5%, about 9%, about 9.5%, about 10%, about 10.5%, about 11%, about 11.5% or about 12%.

Accordingly in an embodiment the modified release coating comprises ethyl cellulose and a water-soluble polysaccharide (particularly pectin) wherein the water-soluble polysaccharide (WSP) is present in an amount of 0.1% to about 10% by weight, based on the dry weight of the modified release coating. Suitably the WSP is present in an amount of from about 0.5% to about 10%, for example about 0.5% to about 5%, about 1% to about 3%, suitably about 2% based on the total dry weight of the modified release coating. In this embodiment the WSP is preferably pectin. In this embodiment the modified release composition suitably further comprises a plasticizer. Suitable plasticizers include these described above in relation to Surelease™. Suitably the weight gain of the composition due to application of the modified release coating in this embodiment is in the range of from 1 to 30% (for example from: 3% to 25%; 1% to 20%; 5% to 15%; 8% to 14%; 10% to 12%; 12% to 18%; or 16% to 18%, suitably the weight gain is about 11%, about 11.5%, or about 17%).

### Sub-Coating

It has been found that sub-coating a core comprising cyclosporin prior to applying a modified release coating provides advantages properties to the composition. The presence of a sub-coating has been found to enhance the dissolution properties of the modified release compositions according to the invention. In particular the presence of a sub-coating has been found to increase the rate of release of cyclosporin from the composition and also to increase the amount of cyclosporin released in a set time period compared to compositions prepared without using a sub-coating. The presence of a sub-coating has also been found to reduce batch-to-batch variability, particularly when the core is in the form of a minibead. A sub-coating may therefore reduce intra- and inter-patient variability.

Accordingly the modified release composition may be a modified release composition comprising cyclosporin, wherein the composition further comprises a first coating and a second coating outside the first coating; and wherein

the first coating is or comprises a water-soluble cellulose ether or a water-soluble derivative of a cellulose ether; and

the second coating is or comprises a modified release coating.

Suitably in this embodiment the first coating (sub-coating) is applied to a core comprising cyclosporin. In a particular embodiment the core is or comprises cyclosporin in a polymeric matrix, particularly a water-soluble polymer matrix. Still more particularly the core comprises a hydrogel forming polymer matrix and cyclosporin. Such cores are described in more detail below. The first coating is suitably a coating on the outer surface of the core.

The first coating is water-soluble, suitably the first coating is soluble in the environment of the lower GI tract following oral administration of the composition, for example the first coating is soluble in water at a pH of 5.5 or more, suitably at a pH of 6.5 or more.

The first coating is or comprises a water-soluble cellulose ether or a water-soluble derivative of a cellulose ether or a combination of two or more such materials. The water-soluble derivative of a cellulose ether may be or comprise a water-soluble ester of a cellulose ether. Accordingly in an embodiment the first coating is or comprises a water-soluble cellulose ether or a water-soluble ester of a cellulose ether. Preferably the first coating is or comprises a water-soluble cellulose ether. In some embodiments the first coating may be or comprise a water-soluble derivative of a cellulose ether, for example a water-soluble ester of a cellulose ether.

Suitably the material of the first coating (i.e. the sub-coating) is different to the modified release coating on the composition. For example, where the first coating is or comprises a water-soluble cellulose ether of derivative thereof, the major component(s) (e.g. more than 50%) of the modified release coating is or comprises a different polymer to that of the first coating. Accordingly, the first and second coatings suitably provide two layers of material as part of the composition. It is to be understood that when the modified release coating comprises a mixture of components, minor components of the outer modified release coating may the same as the material of the sub-coating. By way of example, when the first coating is or comprises HPMC and the modified release coating (second coating) comprises ethyl cellulose, the ethyl cellulose may optionally further comprise a minor amount (e.g. less than 50%, 40%, 30% or 30%) of the first coating material, HPMC in this example. In such embodiments the sub-coat and the modified release coating are considered to be different.

The water-soluble cellulose ether may be a water-soluble cellulose ether selected from an alkyl cellulose, for example methyl cellulose, ethyl methyl cellulose; a hydroxyalkyl cellulose, for example hydroxyethyl cellulose (available as Cellosize™ and Natrosol™) hydroxypropyl cellulose (available as Klucel™) or hydroxymethyl cellulose; a hydroxyalkyl alkyl cellulose, for example hydroxyethyl methyl cellulose (NEMC), hydroxypropyl methyl cellulose (available as Methocel™, Pharmacoat™, Benecel™) or ethyl hydroxyethyl cellulose (EHEC); and a carboxyalkyl cellulose, for example carboxymethyl cellulose. Suitably the water-soluble cellulose ether may, for example be selected from methyl cellulose, hydroxyethyl cellulose, hydroxypropyl cellulose and hydroxypropylmethyl cellulose.

The polymer of the first coat, for example a water-soluble cellulose ether, may be a low viscosity polymer which is suitable for application as a film or coating to the composition. The viscosity of the polymer may be from about 2 to about 60 mPa·s, for example a viscosity of: about 2 to about 20 mPa·s; about to 2 to about 8 mPa·s; more suitably a viscosity of about 4 to about 10 mPa·s, for example about 4 to about 6 mPa·s. Alternatively, the viscosity of the polymer may fall outside any or all of the just-mentioned ranges, for example be above 20 mPa·s. The viscosity of the polymer may be determined by measuring the viscosity of a 2% solution of the polymer in water at 20° C. using a Ubbelode viscometer using ASTM standard methods (D1347 and D2363).

The first coat may be or comprise a water-soluble hydroxypropylmethyl cellulose (HPMC or hypromellose). HPMC is prepared by modifying cellulose to substitute hydroxy groups with methoxy and hydroxypropyl groups. Each anhydroglucose unit in the cellulose chain has three hydroxyl groups. The amount of substituent groups on the anhydroglucose units may be expressed as the degree of substitution. If all three hydroxyl groups on each unit are substituted, the degree of substitution is 3. The number of substituent groups on the ring determines the properties of the HPMC. The degree of substitution may also be expressed as the weight % of the methoxy and hydroxypropyl groups present. Suitably the HPMC has from about 19 to about 30% methoxy substitution and from about 7 to about 12% hydroxypropyl substitution. Particularly the HPMC has 25 to 30% methoxy substitution and 7 to 12% hydroxypropyl substitution. Suitably the HPMC is a low viscosity HPMC which is suitable for application as a film or coating to the composition. The viscosity of the HPMC is suitably from about 2 to 60 mPa·s, for example about 2 to about 20 mPa·s, more suitably a viscosity of about 4 to about 10 mPa·s. The viscosity of the HPMC is determined by measuring the viscosity of a 2% solution of the HPMC in water at 20° C. using a Ubbelode viscometer using ASTM standard methods (D1347 and D2363). Such HPMC is available as for example Methocel™, for example Methocel™ E, including Methocel™ E5.

When the first coating is or comprises a water-soluble derivative of a cellulose ether, the derivative may, for example be a water-soluble ester of a cellulose ether. Water-soluble esters of cellulose ethers are well known and may comprise esters of a cellulose ether, formed with one or more suitable acylating agent(s). Acylation agents may be, for example suitable acids or acid anhydrides or acyl halides. Accordingly the ester of a cellulose ether may contain a single ester moiety or two or more ester moieties to give a mixed ester. Examples of water-soluble esters of cellulose ethers may be water-soluble phthalate, acetate, succinate, propionate or butyrate esters of a cellulose ether (for example HPMC). Suitably the water-soluble ester of a cellulose ether is a water-soluble phthalate, acetate-succinate, propionate, acetate-propionate or acetate-butyrate ester of a cellulose ether (for example HPMC).

The water-soluble ester of a cellulose ether may be or comprise a water-soluble ester of any of the water-soluble cellulose ethers described above in relation to the sub-coating.

Particular water-soluble esters of cellulose ethers are water-soluble esters of HPMC. Esters of HPMC which are soluble in water at a pH greater than 5.5 may be or comprise hydroxypropyl methylcellulose phthalate (HPMCP), or hydroxypropyl methylcellulose acetate succinate (HPMCAS) in which the presence of ionisable carboxyl groups causes the polymer to solubilize at high pH (>5.5 for the LF grade and >6.8 for the HF grade). These polymers are commercially available from Shin-Etsu Chemical Co. Ltd.

The first coat may comprise or be hypromellose, e.g. it may be made of a mixture of hypromellose, titanium dioxide and polyethylene glycol; the first coat may comprise at least 50 wt % hypromellose and optionally at least 75 wt % hypromellose, e.g. at least 80 wt % or at least 85 wt % or 90 wt % hypromellose. The coating material used to form the first coat may therefore comprise a dry weight percentage of hypromellose mentioned in the preceding sentence.

If it is desired for the first coat to use a mixture of hypromellose, titanium dioxide and polyethylene glycol, commercial products corresponding to such mixtures are available including Opadry White, a product commercialised by Colorcon. More generally, there may be mentioned various products commercialised under the trade name Opadry and Opadry II. Further non limiting examples include Opadry YS-1-7706-G white, Opadry Yellow 03B92357, Opadry Blue 03B90842). These compositions are available as dry film coating compositions that can be diluted in water shortly before use. Opadry and Opadry II formulations comprise a cellulosic film forming polymer (e.g., HPMC and/or HPC), and may contain polydextrose, maltodextrin, a plasticizer (e.g., triacetin, polyethylene glycol), polysorbate 80, a colorant (e.g., titanium dioxide, one or more dyes or lakes), and/or other suitable film-forming polymers (e.g., acrylate-methacrylate copolymers). Suitable OPADRY or OPADRY II formulations may comprise a plasticizer and one or more of maltodextrin, and polydextrose (including but not limited to a) triacetin and polydextrose or maltodextrin or lactose, or b) polyethylene glycol and polydextrose or maltodextrin). Particularly preferred commercial products are Opadry White (HPMC/HPC-based) and Opadry II White (PVA/PEG-based).

The first coating may also be applied as a simple solution comprising water and the polymer of the first coating. For example when the polymer is HPMC, for example Methocel, the first coating may be applied to the core as an aqueous solution or dispersion of the HPMC. Optionally the coating solution may include other solvents such as an alcohol. Alternatively the coating may be applied as a solution or dispersion in a volatile organic solvent.

Suitably the first coating is present in an amount corresponding to a weight gain of the composition due to the first coating of from 0.5% to 40% (for example from 0.5% to 30%; from 0.5% to 20%; from 1% to 25%; from 1 to 20%; from 1% to 15%; from 1% to 6%; from 1% to 4%; from 4% to 6%; from 6% to 10%; from 9% to 15%; or from 12% to 15%) by weight based upon the weight of the composition prior to applying the first coating.

The first coating may be present in an amount corresponding to a weight gain due to the first coating in a range selected from 9 to 30%, suitably 9% to 20%, or particularly 10% to 15% by weight based upon the weight of the composition prior to applying the first coating.

Suitably the first coating (sub-coating) provides a coating thickness on the composition of from about 10 μm to about 1 mm, for example, from about 10 μm to about 500 μm, from about 50 μm to about 1 mm, or about from about 50 μm to about 500 μm. The thickness may therefore be from about 100 μm to about 1 mm, e.g. 100 μm to about 750 μm or about 100 μm to about 500 μm. The thickness may be from about 250 μm to about 1 mm, e.g. about 250 μm to about 750 μm or 250 μm to about 500 μm. The thickness may be from about 500 μm to about 1 mm, e.g. about 750 μm to about 1 mm or about 500 μm to about 750 μm. The thickness may therefore be from about 10 μm to about 100 μm, e.g. from about 10 μm to about 50 μm or about 50 μm to about 100 μm.

It is preferred to dry the composition of the invention before the first coat is applied as is described in more detail below in relation to the coating process.

The second coating is outside the first coating and may be any on the modified release coatings described above. In particular, the second coating is or comprises a pH independent polymer modified release coating described above. For example the second coating may be or comprise an enteric coating or a pH independent coating. The second coating may comprise a mixture of polymers including a polymer degradable by bacterial or other enzymes. In a particular embodiment the second coating comprises ethyl cellulose (for example a Surelease™ coating). In another particular embodiment the second coating comprises ethyl cellulose and a water-soluble polysaccharide, in particular one susceptible to degradation by colonic bacteria, suitably pectin. Accordingly the second coating may comprise the Surelease-pectin mixture described above. The second coating may be or comprise ethyl cellulose (e.g. Surelease™) and a pore former, wherein the pore-former is a water-soluble excipient which acts to enhance the permeability of the coating when placed in an aqueous environment such as that found in the lower GI tract. Suitable pore formers include those described above. In embodiments the second coating does not comprise a pore former, for example, the second coating may comprise ethyl cellulose and no pore former such as pectin.

Accordingly the modified release composition may comprise a core, a first coating and a second coating outside the first coating; and wherein:

the core comprises a polymer matrix and cyclosporin;

the first coating is or comprises a water-soluble cellulose ether, particularly hydroxypropylmethyl cellulose;

the second coating is or comprises a modified release coating, particularly a pH independent modified release coating;

the first coating is present in an amount corresponding to a weight gain due to the first coating in a range selected from: (i) from 1% to 20%; (ii) from 8% to 12%, for example about 10%; (iii) from 4% to 6%, for example about 5%; or (iv) about 6% to about 10%, for example about 7%, about 7.5%, about 8%, about 8.5%, about 9% or about 9.5% by weight based upon the dry weight of the composition prior to applying the first coating; and wherein

the second coating is present in an amount corresponding to a weight gain of the composition due to the second coating selected from (a) from 5 to 40%; (b) from 10% to 12%, for example about 11% or about 11.5%; (c) from 16% to 18%, for example about 17%%; or (d) from about 8% to about 12%, for example about 8.5%, about 9%, about 9.5%, about 10%, about 10.5% or about 11% by weight based upon the weight of the composition prior to applying the second coating.

Suitably in this embodiment the core comprises a water-soluble polymer matrix and cyclosporin. More particularly the core comprises a hydrogel forming polymer matrix and cyclosporin as described in more detail below.

The first and second coatings in this embodiment are suitably any of the first and second coatings described above or below. Accordingly it is intended that the coatings described in this section may be applied to any of the compositions described herein to provide a modified release coating if required. The coatings are particularly useful to provide a modified release coating to the cores comprising a polymer matrix and cyclosporin described in this application.

The presence of a sub-coating, amongst other things, increases the amount of cyclosporin released from the composition during dissolution compared to compositions without a sub-coating. Accordingly the modified release composition comprising cyclosporin, may comprises a first coating (sub coating) and second coating (modified release coating) as described herein; wherein the first coating is present in an amount to provide a % release of the cyclosporin that is higher than a % release of the cyclosporin from a corresponding composition without the first coating throughout a time period from 8 hours to 18 hours, when measured in the two stage dissolution test described herein. For example the sub-coated composition provides a higher % release in the period between 10 hours and 16 hours, suitably between 10 hours and 14 hours and more particularly at about 10 hours, about 12, hours about 14 hours or about 16 hours in the two stage dissolution test. The sub-coated composition of the invention may, for example, provide 2% or higher, 5% or higher, 10% or higher, 20% or higher, or 30% or higher more cyclosporin release at a given time point during the two stage dissolution test compared to the same composition without the subcoating. For example 2 to 30%, particularly 2 to 20% more cyclosporin. In this embodiment it is to be understood that reference to a higher % release refers to an absolute percentage increase. By way of an example if an uncoated composition releases 10% cyclosporin at a particular time point and the coated composition releases 10% more cyclosporin, this means that the coated composition releases 20% cyclosporin at the same time point.

### Outer Barrier or Protective Coating

The compositions described herein may comprise a protective coating. The protective coating may help to protect the modified release coating from damage resulting from, for example formulating the composition into a final dosage form, or during the handling, transport or storage of the composition. The protective coating is suitably applied to the outer surface of the composition. When the composition has a modified release coating, the protective coating is suitably outside the modified release coating. The protective coating may be applied to an outer surface of the composition, for example the protective coating may be applied directly to the modified release coating such that the protective coating is in contact with the modified release coating. Alternatively the protective coating may be separated from the modified release coating by, for example one or more intermediate excipient and/or substance layers between the modified release coating and the protective coating. Optionally the protective coating may be applied to the outer surface of the composition after it has been formulated into a unit dosage form such as a tablet. The protective coating is suitably a water soluble coating which does not adversely affect the release of the cyclosporin from the composition when in use. Suitably the protective coating is or comprises a water-soluble polymer. The protective coating may comprise a water-soluble cellulosic or PVA film-forming polymer. Suitably the protective coating may be or comprise Opadry (HPMC/HPC-based), Opadry II (PVA/PEG-based) or polyvinyl alcohol-polyethylene glycol graft copolymers (Kollicoat IR) as described herein. The protective coating may be present as a layer of from about 2 to about 50 μm. Suitably the protective coating is applied to give a weight-gain of from about 0.5 to about 10%, based upon the weight of the composition prior to applying the protective coating.

### Polymer Matrix Core

Suitably the composition of the invention comprises a core wherein the core comprises a cyclosporin phase and a continuous phase or matrix phase to provide mechanical strength. In embodiments the cyclosporin phase is or comprises a disperse phase within the continuous phase or matrix. The continuous phase or matrix phase suitably comprises a water-soluble polymer matrix and in particular comprises a hydrogel-forming polymer matrix. The core may comprise a polymer matrix wherein the matrix-forming polymer is a hydrogel-forming polymer or a combination thereof. Optionally the core may be coated with a modified release coating or a sub-coating and a modified release coating as described above to provide a particular modified release profile.

The cyclosporin may be present as a disperse hydrophobic phase within the hydrogel-forming polymer matrix (continuous phase or aqueous phase) of the core. For example the disperse phase may comprise a lipid and cyclosporin. The cores may be prepared by dispersing the cyclosporin phase within the aqueous phase to form a colloid and then causing the composition to solidify (gel), thereby immobilising the cyclosporin within the hydrogel-forming polymer matrix.

The core may have the form of a solid colloid, the colloid comprising a continuous phase and a disperse phase, wherein the continuous phase is or comprises the hydrogel forming polymer and the disperse phase is or comprises cyclosporin. The disperse phase may comprise a vehicle containing the cyclosporin, for example containing it as a solution or a suspension. The vehicle may be hydrophobic, and may comprise or be a solution of cyclosporin or a suspension of cyclosporin. The disperse phase may by way of example be liquid, semi-solid or solid.

The core may have the characteristics of a dried colloid in which cyclosporin is dispersed within the hydrogel-forming polymer matrix. Thus, the core may have the form of a dried colloid, the colloid comprising a continuous phase and a disperse phase, wherein the continuous phase is or comprises the hydrogel-forming polymer and the disperse phase is or comprises cyclosporin. The disperse phase may comprise a vehicle containing the cyclosporin, for example containing it as a solution or a suspension. The vehicle may be hydrophobic, and may comprise or be a solution of cyclosporin or a suspension of cyclosporin. The disperse phase may by way of example be liquid, semi-solid or solid. The dried colloid may be a dried emulsion, i.e. the core may have the characteristics of a dried colloid.

Such cores comprising a water-soluble polymer, particularly a hydrogel-forming polymer and a disperse phase comprising cyclosporin are described in more detail below.

### Continuous Phase Polymer Matrix (Aqueous Phase)

This section of the specification relating to the polymer matrix recites amounts of constituents in terms of percent by weight of the formulation. In the context of this section of the specification, what is meant is percent by weight of the dry weight of the core, i.e.

excluding coating(s).

It will be recalled that the core may comprise a matrix or continuous phase and optionally, but not necessarily, also a disperse phase or discontinuous phase. Suitably the continuous phase of the core is or comprises a hydrogel-forming polymer. A hydrogel forming polymer is a polymer capable of forming a hydrogel. A hydrogel may be described as a solid or semi-solid material, which exhibits no flow when at rest, comprising a network (matrix) of hydrophilic polymer chains that span the volume of an aqueous liquid medium.

The core may comprise a hydrogel-forming polymer selected from the group consisting of: gelatin; agar; agarose; pectin; carrageenan; chitosan; alginate; starch; xanthan gum; gum Arabic; guar gum; locust bean gum; polyurethane; polyether polyurethane; cellulose; cellulose ester, cellulose acetate, cellulose triacetate; cross-bonded polyvinyl alcohol; polymers and copolymers of acrylic acid, hydroxyalkyl acrylates, hydroxyethyl acrylate, diethylene glycol monoacrylate, 2-hydroxypropylacrylate, 3-hydroxypropyl acrylate; polymers and copolymers of methacrylic acid, hydroxyethyl methacrylate, diethyleneglycol monomethacrylate, 2-hydroxypropyl methacrylate, 3-hydroxypropyl methacrylate, dipropylene glycol monomethylacrylate; vinylpyrrolidone; acrylamide polymers and copolymers, N-methylacrylamide, N-propylacrylamide; methacrylamide polymers and copolymers, N-isopropylmethacrylamide, N-2-hydroxyethylmethacrylamide; and vinyl pyrrolidone; and combinations thereof. In specific embodiments binary or tertiary etc combinations of any of the above substances are foreseen.

In a further embodiment the hydrogel-forming polymer is selected from the group consisting of gelatin, agar, a polyethylene glycol, starch, casein, chitosan, soya bean protein, safflower protein, alginates, gellan gum, carrageenan, xanthan gum, phthalated gelatin, succinated gelatin, cellulosephthalate-acetate, oleoresin, polyvinylacetate, hydroxypropyl methyl cellulose, polymerisates of acrylic or methacrylic esters and polyvinylacetate-phthalate and any derivative of any of the foregoing; or a mixture of one or more such a hydrogel forming polymers

The hydrogel-forming polymer may also be referred to as a hydrocolloid i.e. a colloid system wherein the colloid particles are dispersed in water and the quantity of water available allows for the formation of a gel. In embodiments it is preferred to use reversible hydrocolloids preferably thermo-reversible hydrocolloids (e.g. agar, agarose, gelatin etc) as opposed to irreversible (single-state) hydrocolloids. Thermo-reversible hydrocolloids can exist in a gel and sol state, and alternate between states with the addition or elimination of heat. Gelatin, agar and agarose are thermo-reversible, rehydratable colloids and are particularly preferred. Gelatin derivatives such as, for example, succinated or phthalated gelatins are also contemplated. Thermoreversible hydrocolloids which may be used according to the invention, whether individually or in combination, include those derived from natural sources such as, for example, carrageenan (extracted from seaweed), gelatin (extracted from bovine, porcine, fish or vegetal sources), agar (from seaweed), agarose (a polysaccharide obtained from agar) and pectin (extracted from citrus peel, apple and other fruits). A non-animal based hydrocolloid may be preferred for certain applications e.g. administration to vegetarians or to individuals not wishing to ingest animal products for religious or health reasons. In relation to the use of carrageenan, reference is made to US patent application 2006/0029660 A1 (Fonkwe et al), the entirety of which is incorporated herein by reference. The hydrogel-forming polymer may comprise or be a combination of gelatin with one or more other thermoreversible hydrocolloids, e.g. with one or more other of the thermoreversible hydrocolloids just listed. The hydrogel-forming polymer may comprise or be a combination of gelatin with agar; optionally, at least one further thermoreversible hydrocolloid may be included in the combination, for example one just listed.

Thermo-reversible colloids present a benefit over other hydrogel-forming polymers. Gelation or hardening of thermo-reversible colloids occurs by cooling the colloid, e.g. in a liquid cooling bath or by air flow. Gelation of other hydrogel-forming polymers, which is chemically driven, can lead to leakage of the composition contents into the gelation medium as the hardening process can take time to occur. Leakage of the content of the composition may lead to an inaccurate quantity of the active ingredient within the composition. Thermo-reversible colloids are also known as thermo-reversible gels, and it is therefore preferred that the hydrogel former be a thermo-reversible gelling agent.

Another term which may be applied to hydrogel formers which are advantageous is “thermotropic”: a thermotropic gelling agent (which the reader will infer is preferred as a hydrogel former used in the invention) is one caused to gel by a change in temperature and such gelling agents are able to gel more rapidly than those whose gelling is chemically induced, e.g. ionotropic gelling agents whose gelling is induced by ions, for example chitosan. In embodiments of the invention, therefore, the hydrogel former is a thermotropic gel-forming polymer or a combination of such polymers.

The manufacture of the composition to prepare a core may require that the hydrogel-forming polymer be present as a solution, which is preferably an aqueous solution. The hydrogel-forming polymer represents between 5% and 50%, preferably between 10% and 30%, still more preferably between 15% and 20% by weight of the aqueous phase during manufacture as described herein. In addition the hydrogel-forming polymer may comprise 8 to 35%, (for example 15-25%, preferably 17-18%) hydro-gel forming polymer; 65%-85% (preferably 77-82%) of water plus, optionally, from 1-5% (preferably 1.5 to 3%) sorbitol. When present surfactant (e.g. anionic surfactant) in the aqueous phase pre-mix may be present in an amount of 0.1 to 5% (preferably 0.5 to 4%) wherein all parts are by weight of the aqueous phase.

In embodiments the composition comprises at least 25%, suitably at least 40% by weight based upon the dry weight of the composition of the hydrogel-forming polymer. For example the hydrogel-forming polymer is present form 25 to 70%, for example 40 to 70% suitably 45 to 60% of the composition, wherein the % is by weight based upon the dry weight of the composition.

In embodiments the hydrogel-forming polymer is a pharmaceutically acceptable polymer.

In certain embodiments the hydrogel-forming polymer is gelatin. In certain embodiments the hydrogel-forming polymer comprises gelatin. In certain embodiments the gelatin comprises at least 40%, for example 40 to 70% suitably 45 to 60% of the composition, wherein the % is by weight based upon the dry weight of the composition.

The hydrogel-forming polymer may optionally comprise a plasticiser for example sorbitol or glycerine, or a combination thereof. In particular one or more plasticisers may be combined with gelatin.

In embodiments in which the hydrogel-forming polymer comprises or is, reference is hereby made to “Bloom strength”, a measure of the strength of a gel or gelatin developed in 1925 by O. T. Bloom. The test determines the weight (in grams) needed by a probe (normally with a diameter of 0.5 inch) to deflect the surface of the gel 4 mm without breaking it. The result is expressed in Bloom (grades) and usually ranges between 30 and 300 Bloom. To perform the Bloom test on gelatin, a 6.67% gelatin solution is kept for 17-18 hours at 10° C. prior to being tested.

When the hydrogel-forming polymer comprises or is gelatin the bloom strength of the gelatin may be in the range of 125 Bloom to 300 Bloom, 200 Bloom to 300 Bloom and preferably 250 Bloom to 300 Bloom. It should be appreciated that higher bloom strength gelatin can be replaced by lower bloom strength gelatin at higher concentrations.

According to the invention, in embodiments in which the hydrogel-forming polymer matrix comprises or is gelatin, the gelatin may be sourced by a variety of means. For example, it can be obtained by the partial hydrolysis of collagenous material, such as the skin, white connective tissues, or bones of animals. Type A gelatin is derived mainly from porcine skins by acid processing, and exhibits an isoelectric point between pH 7 and pH 9, while Type B gelatin is derived from alkaline processing of bones and animal (bovine) skins and exhibits an isoelectric point between pH 4.7 and pH 5.2. Type A gelatin is somewhat preferred. Gelatin for use in the invention may also be derived from the skin of cold water fish. Blends of Type A and Type B gelatins can be used in the invention to obtain a gelatin with the requisite viscosity and bloom strength characteristics for bead manufacture.

Lower temperature gelatin (or gelatin derivatives or mixtures of gelatins with melting point reducers) or other polymer matrices able to be solidified at lower temperatures (e.g. sodium alginate) may also be used. It is therefore believed that polymer which comprises or is low temperature gelatin is a preferred matrix polymer.

In embodiments in which the polymer comprises or is gelatin, the starting gelatin material is preferably modified before manufacture to produce “soft gelatin” by the addition of a plasticizer or softener to the gelatin to adjust the hardness of the composition of the invention. The addition of plasticizer achieves enhanced softness and flexibility as may be desirable to optimise dissolution and/or further processing such as, for example, coating. Useful plasticizers of the present invention for combination with gelatin or another hydrogel-forming polymer include glycerine (1,2,3-propanetriol), D-sorbitol (D-glucitol), sorbitol BP (a non-crystallizing sorbitol solution) or an aqueous solution of D-sorbitol, sorbitans (e.g. Andidriborb 85/70), mannitol, maltitol, gum arabic, triethyl citrate, tri-n-butyl citrate, dibutylsebacate. Other or similar low molecular weight polyols are also contemplated for example ethylene glycol and propylene glycol. Polyethylene glycol and polypropylene glycol may also be used although these are less preferred. Glycerine and D-sorbitol may be obtained from the Sigma Chemical Company, St. Louis, Mo. USA or Roquette, France. Some active agents and excipients included for other functions may act as plasticisers.

Softeners or plasticisers, if utilized, can be ideally incorporated in a proportion rising to 30%, preferably up to 20% and more preferably up to 10% by dry weight of the composition of the invention, even more preferably between 3 and 8%, and most preferably between 4% and 6%.

Although not essential, the hydrogel-forming polymer matrix may also optionally contain a disintegrant where it is particularly desired to enhance the rate of disintegration of the composition of the invention. Examples of disintegrants which may be included are alginic acid, croscarmellose sodium, crospovidone, low-substituted hydroxypropyl cellulose and sodium starch glycolate.

A crystallisation inhibitor (e.g. approximately 1% by dry weight of the composition) may also be included in the composition of the invention. An example is hydroxy propyl/methyl cellulose (HPC or HPMC, hypromellose etc) which may play other roles such as, for example, emulsifier.

In another embodiment, the hydrogel forming polymer matrix is chitosan which can exist in the form of biogels with or without additives as described e.g. in U.S. Pat. No. 4,659,700 (Johnson & Johnson); by Kumar Majeti N. V. Ravi in Reactive and Functional Polymers, 46, 1, 2000; and by Paul et al. in ST.P. Pharma Science, 10, 5, 2000 the entirety of all 3 of which is incorporated herein by reference. Chitosan derivatives e.g. thiolated entities are also contemplated.

The hydrogel-forming polymer matrix may be a non-hydrocolloid gum. Examples are the cross-linked salts of alginic acid. For example, aqueous solutions of sodium alginate gums extracted from the walls of brown algae have the well-known property of gelling when exposed to di- and trivalent cations. A typical divalent cation is calcium, often in the form of aqueous calcium chloride solution. It is preferred in this embodiment that the cross-linking or gelling have arisen through reaction with such a multivalent cation, particularly calcium.

The hydrogel-forming polymer matrix may have a low water content, therefore the composition may have a low water content. As described below during manufacture of a core the disperse phase comprising cyclosporin is mixed with an aqueous solution of the hydrogel-forming polymer and composition is gelled, for example to provide cores which are minibeads. Suitably the cores are dried following formation to reduce the water content present in the core.

In certain embodiments the composition does not comprise compounds containing a disulphide bond. In embodiments the hydrogel-forming polymer does not comprise compounds containing a disulphide bond.

The hydrogel-forming polymer matrix forming the continuous phase of the core (aqueous phase) may further comprise a surfactant. Surfactants which may be used in the composition are described in the section “surfactants” below.

Surfactant which may be present in the continuous aqueous phase of the core include, for example, a surfactant selected from the group consisting of: cationic; amphoteric (zwitterionic); anionic surfactants, for example perfluoro-octanoate (PFOA or PFO), perfluoro-octanesulfonate (PFOS), sodium dodecyl sulfate (SDS), ammonium lauryl sulfate, and other alkyl sulfate salts, sodium laureth sulfate, also known as sodium lauryl ether sulfate (SLES) and alkyl benzene sulfonate; and non-ionic surfactants for example perfluorocarbons, polyoxyethyleneglycol dodecyl ether (e.g. Brij such as, for example, Brij 35), Myrj (e.g. Myrj 49, 52 or 59), Tween 20 or 80 (also known as Polysorbate) (Brij, Myrj and Tween products are available commercially from Croda), poloxamers which are nonionic triblock copolymers composed of a central hydrophobic chain of polyoxypropylene (poly(propylene oxide)) flanked by two hydrophilic chains of polyoxyethylene (poly(ethylene oxide)), or a combination of the foregoing. In particular, the surfactant may be selected from, or comprise, anionic surfactants and combinations thereof, the anionic surfactants optionally being those mentioned in this paragraph. A particular class of surfactant comprises sulfate salts. A preferred anionic surfactant in the aqueous phase is SDS. Mixtures of further surfactants are also contemplated, e.g. mixtures comprising perfluorocarbons.

In embodiments of the invention, the core comprises a hydrophilic surfactant which, without being bound by theory, is believed at least partially to partition the aqueous phase (polymer matrix).

Such surfactants intended for such inclusion in the aqueous phase of the core are preferably readily diffusing or diffusible surfactants to facilitate manufacturing and processing of the composition of the invention. The surfactant may have an HLB of at least 10 and optionally of at least 15, e.g. at least 20, or at least 30 and optionally of 38-42, e.g. 40. Such surfactants can be of any particular type (ionic, non-ionic, zwitterionic) and may comprise as a proportion of dry weight of the composition from 0.1% to 6%, e.g. 0.1% to 5%. 0.1% to 4% or 0.1% to 3%, more preferably in a proportion of at least 1% and in particular between 1.0 and 4.5 or 5%, ideally within or just outside the 2-4% range, for example from 2 to 3% or approximately 2% or approximately 4%.

Unless otherwise stated or required, all percentages and ratios are by weight.

In one embodiment the anionic surfactant may be an anionic surfactant selected from alkyl sulfates, carboxylates or phospholipids, or combinations thereof.

The physical form of the surfactant at the point of introduction into the aqueous phase during preparation of the core plays a role in the ease of manufacture of the core.

As such, although liquid surfactants can be employed, it is preferred to utilize a surfactant which is in solid form (e.g. crystalline, granules or powder) at room temperature, particularly when the aqueous phase comprises gelatin.

In general, mixtures of surfactants can be utilised e.g. to achieve optimum long term stability of the composition of the invention with shorter chain surfactants in general facilitating shorter term stability (an aid to processing) and longer chain surfactants facilitating longer term stability (an aid to shelf life). In some embodiments, shorter chain surfactants have up to C₁₀ alkyl (e.g. C₆-C₁₀ alkyl) as the hydrophobic portion of the surfactant whilst longer chain surfactants have C₁₀ or higher alkyl (e.g. C₁₀-C₂₂ alkyl) as the hydrophobic portion of the surfactant. It is envisaged that C₁₀ alkyl surfactants may facilitate processing or facilitate prolongation of shelf life, or both, depending on the identity of the other excipients and of the active principle(s). Higher alkyl may in particular implementations of the invention be C₁₁-C₂₂ or C₁₂-C₂₂ alkyl, and in some embodiments has a length of no greater than C₁₈.

### Disperse Phase

The polymer matrix of the core described above (for example a hydrogel-forming polymer) further comprises a disperse phase. Suitably the disperse phase is or comprises cyclosporin. In embodiments the disperse phase comprises cyclosporin. In such embodiments the cyclosporin is preferably soluble in the disperse phase. Embodiments wherein the cyclosporin is soluble in the disperse phase are preferred, because such compositions release the cyclosporin in a solubilised form, which may enhance the therapeutic effect of the drug at the site of release, for example by enhancing absorption into the colonic mucosa.

In embodiments the cyclosporin is or is comprised in the disperse phase.

Suitably the disperse phase comprises an oil phase and optionally the oil phase is or comprises a liquid lipid and optionally a solvent miscible therewith. Cyclosporin, may be present in the oil phase. Suitably the cyclosporin is soluble in the oil phase.

The disperse phase may comprise a combination of oils. The liquid lipid may be a short-, medium- or long-chain triglyceride composition, or a combination thereof. A medium chain triglyceride(s) (MCT) comprises one or more triglycerides of at least one fatty acid selected from C₆, C₇, C₈, C₉, C₁₀ C₁₁ and C₁₂ fatty acids. It will be understood that commercially available triglyceride, in particular MCT, compositions useful in the invention are mixtures derived from natural products and usually or always contain minor amounts of compounds which are not MCTs; the term “medium chain triglyceride composition” is therefore to be interpreted to include such compositions. A short chain triglyceride(s) comprises one or more triglycerides of at least one short chain fatty acid selected from C₂-C₅ fatty acids. A long chain triglyceride(s) comprises one or more triglycerides of at least one long chain fatty acid having at least 13 carbon atoms.

The liquid lipid may be or comprise triglycerides and/or diglycerides. Such glycerides may be selected from medium chain glycerides or short chain triglycerides or a combination thereof.

The liquid lipid may be a caprylic/capric triglyceride, i.e. a caprylic/capric triglyceride composition (which it will be understood may contain minor amounts of compounds which are not caprylic/capric triglycerides).

Said solvent which is optionally included in an oil phase may be miscible with both the liquid lipid and with water. Examples of suitable solvents are 2-(2-ethoxyethoxy)ethanol available commercially under trade names Carbitol™, Carbitol cellosolve, Transcutol™, Dioxitol™, Poly-solv DE™, and Dowanal DE™; or the purer Transcutol™ HP (99.9). Transcutol P or HP, which are available commercially from Gattefosse, are preferred. Another possible solvent is poly(ethylene glycol). PEGs of molecular weight 190-210 (e.g. PEG 200) or 380-420 (e.g. PEG 400) are preferred in this embodiment. Suitable PEGs can be obtained commercially under the name “Carbowax” manufactured by Union Carbide Corporation although many alternative manufacturers or suppliers are possible.

The disperse phase, may represent from 10-85% by dry weight of the core.

As discussed above the disperse phase may be an oil phase comprising any pharmaceutically suitable oil, e.g. a liquid lipid. The oil phase may be present as oil drops. In terms of dry weight of the core, the oil phase may comprise a proportion from 10% to 85%, e.g. 15% to 50%, for example 20% to 30% or from 35% to 45%. The term “oil” means any substance that is wholly or partially liquid at ambient temperature or close-to-ambient temperature e.g. between 10° C. and 40° C. or between 15° C. and 35° C., and which is hydrophobic but soluble in at least one organic solvent. Oils include vegetable oils (e.g. neem oil) and petrochemical oils.

Oils which may be included in the oil phase include poly-unsaturated fatty acids such as, for example, omega-3 oils for example eicosapentanoic acid (EPA), docosohexaenoic acid (DHA), alpha-linoleic acid (ALA), conjugated linoleic acid (CLA). Preferably ultrapure EPA, DHA or ALA or CLA are used e.g. purity up to or above 98%. Omega oils may be sourced e.g. from any appropriate plant e.g. sacha inchi. Such oils may be used singly e.g. EPA or DHA or ALA or CLA or in any combination. Combinations of such components including binary, tertiary etc combinations in any ratio are also contemplated e.g. a binary mixture of EPA and DHA in a ratio of 1:5 available commercially under the trade name Epax 6000. The oil part of the oil phase may comprise or be an oil mentioned in this paragraph.

Oils which may be included in the oil phase are particularly natural triglyceride-based oils which include olive oil, sesame oil, coconut oil, palm kernel oil, neem oil. The oil may be or may comprise saturated coconut and palm kernel oil-derived caprylic and capric fatty acids and glycerin e.g. as supplied under the trade name Miglyol™ a range of which are available and from which one or more components of the oil phase of the invention may be selected including Miglyol™ 810, 812 (caprylic/capric triglyceride); Miglyol™ 818: (caprylic/capric/linoleic triglyceride); Miglyol™ 829: (caprylic/capric/succinic triglyceride; Miglyol™ 840: (propylene glycol dicaprylate/dicaprate). Note that Miglyol™ 810/812 are MCT compositions which differ only in C₈/C₁₀-ratio and because of its low C₁₀-content, the viscosity and cloud point of Miglyol™ 810 are lower. The Miglyol™ range is available commercially from Sasol Industries. As noted above, oils which may be included in the oil phase need not necessarily be liquid or fully liquid at room temperature. Waxy-type oils are also possible: these are liquid at manufacturing temperatures but solid or semi-solid at normal ambient temperatures. The oil part of the oil phase may comprise or be an oil mentioned in this paragraph.

Alternative or additional oils which may be included in the oil phase according to the invention are other medium chain triglyceride compositions such as for example Labrafac™ Lipophile manufactured by Gattefosse in particular product number WL1349. Miglyol™ 810, 812 are also medium chain triglyceride compositions.

Accordingly the oil phase may be or comprise medium chain mono-di- or tri-glycerides.

The medium chain glyceride(s) (e.g. mono- di- or tri-glyceride(s)) mentioned herein are those which comprise one or more triglycerides of at least one fatty acid selected from fatty acids having 6, 7, 8, 9, 10, 11 or 12 carbon atoms, e.g. C₈-C₁₀ fatty acids.

Other possible (alternative or additional) oils include linoleoyl macrogolglycerides (polyoxylglycerides) such as, for example, Labrafil (e.g. product number M2125CS by Gattefosse) and caprylocaproyl macrogolglycerides such as, for example, Labrasol by Gattefosse.

The oil phase may further comprise one or more surfactants as described below under the section “surfactants”. For example the oil phase may comprise one or more non-ionic or amphoteric surfactants. Particularly the oil phase may comprise a one or more non-ionic surfactant listed under “surfactants” below. The presence of a surfactant in the oil phase may also provide enhanced solubilisation of the cyclosporin (i.e. act as a solubiliser) and/or may provide enhance emulsification when the disperse phase is mixed with the aqueous polymer phase during preparation of the core (i.e. act as an emulsifier).

Surfactant in the oil phase may for example include polyethoxylated castor oils (polyethylene glycol ethers) which can be prepared by reacting ethylene oxide with castor oil. Commercial preparations may also be used as a surfactant/solubilizer e.g. those commercial preparations which contain minor components such as, for example, polyethyelene glycol esters of ricinoleic acid, polyethyelene glycols and polyethyelene glycol ethers of glycerol. A preferred example is Kolliphor™ EL, previously known as Cremophor™ EL. Another surfactant which may be present in the oil phase is for example a phospholipid.

In embodiments the surfactant in the oil phase may be or comprise a non-ionic surfactant selected from sorbitan-based surfactants, PEG-fatty acids, glyceryl fatty acids, or poloxamers.

Within embodiments, the HLB of the oil may be in the range 0-10 (optionally 1-8, e.g. 1-6 and sometimes 1-5).

In one embodiment the oil phase comprises an oil with an HLB in the range 0-10 (preferably 1-5) and a surfactant (suitably a non-ionic surfactant) with an HLB in the range 1-20 and optionally 1 to 15.

In another embodiment the oil phase comprises an oil with an HLB in the range 0-10 (preferably 1-5) and a surfactant (suitably a non-ionic surfactant) with an HLB in the range 10-20 and optionally 11-20 (preferably 11-15).

In another embodiment the oil phase comprises an oil and a surfactant (suitably a non-ionic surfactant) wherein the oil and the surfactant both have an HLB in the range 0-10. For example the oil has an HLB of 1-5, for example 1 to 4 or 1-2 and the surfactant has an HLB 2-8, for example 3-7, 2-6, or 3-4).

Suitable oils with a low HLB (HLB less than 10) include medium chain triglycerides, linoleoyl macrogolglycerides (polyoxylglycerides), caprylocaproyl macrogolglycerides and caprylic/capric triglyceride. In terms of commercial products, particularly preferred oils in the lower HLB range are Labrafac™ Lipophile (e.g. 1349 WL), Labrafil, Labrasol, Captex 355 and Miglyol 810.

One example of a surfactant with high HLB which may be used in a low HLB oil includes polyethoxylated castor oils (polyethylene glycol ethers), for example the commercial product Kolliphor™ EL.

In an embodiment the oil phase comprises of a surfactant of high HLB and an oil of low HLB in a ratio of 1-4:1 by weight, e.g. 1.2-3.0:1 by weight, preferably 1.5-2.5:1 by weight and most preferably 1.8-2.2:1 by weight (high HLB:low HLB) advantageously stabilizes the emulsion before and after immobilization of the oil droplets in the aqueous phase during the preparation of the cores. In this context “stabilize” means in particular that the embodiment improves dissolution and/or dispersion of the composition in vitro. In this embodiment “high” HLB is generally intended above 10, preferably from 10-14, more preferably between 12 and 13. By “low” HLB is generally intended below 10, preferably in the range 1 to 4, more preferably 1 to 2.

It is to be understood that the oil phase in the embodiments above may further comprise or more solvents (co-solvents), for example 2-(2-ethoxyethoxy)ethanol or low molecular weight PEG as mentioned above.

A particular oil phase comprises an oil (low HLB), a high HLB non-ionic surfactant and a co-solvent. For example the following three commercial products: Transcutol P or HP (as co-solvent), Miglyol 810 (as oil) and Kolliphor™ EL (surfactant). Miglyol has a low HLB and Kolliphor™ EL has a high HLB. An oil phase may therefore comprise or consist of a combination of the following and optionally a pharmaceutically active ingredient: 2-ethoxyethanol, an MCT and particularly a caprylic/capric triglyceride formulation, and a polyethoxylated castor oil.

The cyclosporin is preferably soluble in the oil phase. As discussed below in relation to preparation of the core, the cyclosporin is suitably dissolved in the oil phase and the oil phase in mixed with an aqueous phase comprising the hydrogel forming polymer.

The disperse phase (oil phase) may be or comprise a glyceride composition, optionally wherein the disperse phase is or comprises a fatty acid monoglyceride, diglyceride or triglyceride or a combination thereof, or the disperse phase is or comprises a caprylic/capric triglyceride composition.

In embodiments the disperse phase may be or comprise a surfactant. Suitable surfactants include surfactants comprising a hydrophobic chain and a hydrophilic chain can be selected from the group consisting of: macrogol esters; macrogol ethers; diblock copolymers; triblock copolymers; and amphiphilic polymers. Macrogol esters which are suitable for use in the present invention are macrogol esters of fatty acids having at least 6 carbon atoms and optionally at least 10 carbon atoms, and particularly of at least 12 carbon atoms; some fatty acids have no more than 22 carbon atoms, for example C₁₀-C₂₀, C₁₂-C₂₀ or C₁₅-C₂₀ fatty acids. The fatty acids may be saturated or unsaturated but are in particular saturated. To be mentioned are macrogol 25 cetostearyl ether (Cremophor™ A25); macrogol 6 cetostearyl ether (Cremophor™ A6); macrogol glycerol ricinoleate 35 (Kolliphor™ EL); macrogol-glycerol hydroxystearate 40 (Kolliphor™ RH 40); macrogol-15-hydroxystearate (polyoxyl-15-hydroxystearate US Pharmacopoeia and National Formulary, European Pharmacopoeia, e.g. Kolliphor HS 15, previously known as Solutol™ HS 15). Examples of macrogol ethers which are suitable for use in the present invention are macrogol ethers of fatty alcohols having at least 6 carbon atoms and optionally at least 10 carbon atoms, and particularly of at least 12 carbon atoms; some fatty alcohols have no more than 22 carbon atoms, for example C₁₀-C₂₀, C₁₂-C₂₀ or C₁₅-C₂₀ fatty alcohols. The fatty alcohols may be saturate or unsaturated but are in one embodiment saturated. Kolliphor™ HS 15 is obtained by reacting 15 moles of ethylene oxide with 1 mole of 12-hydroxy stearic acid; the surfactant may therefore be or comprise a surfactant obtainable by (having the characteristics of a surfactant obtained by) reacting 10-25 moles of ethylene oxide with 1 mole of 12-hydroxy stearic acid; the number of moles of ethylene oxide may, from 12-25 and optionally from 15-20, e.g. 15 or 20.

Kolliphor™ HS 15 consists of polyglycol mono- and di-esters of 12-hydroxystearic acid and about 30% of free polyethylene glycol. The main components of the ester part have the following chemical structures:

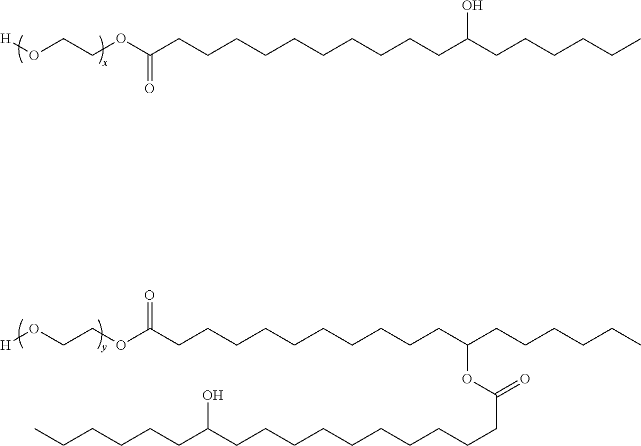

where x and y are integers and a small part of the 12-hydroxy group can be etherified with polyethylene glycol.

A disperse phase which is or comprises a surfactant may enhance the absorption of cyclosporin into the tissue of the GIT, for example by forming self-assembly structures, such as micelles, which are associated with the cyclosporin and thus present the drug to the mucosa tissue of the GI tract in a form which enhances uptake/absorption in the tissue.

The term “self-assembly structure” refers to any type of micelle, vesicle, microemulsion, lyotropic phase, laminar or other self-organised structure that forms spontaneously in the presence of an aqueous environment, or combination thereof. As is known, such self-assembly structures form when a self-assembly structure-forming substance, e.g. comprising or consisting of a surfactant, is present above a certain critical concentration. The term includes, for example, micelles, inverted micelles and liposomes, and combinations thereof. The self-assembly structures referred to in this specification may comprise, or be, micelles. More information on self-assembly structures can be found in “Dynamics of Surfactant Self-assemblies Micelles, Microemulsions, Vesicles and Lyotropic Phases” by Raoul Zana, particularly Chapter 1, all of which is incorporated herein by reference. The release of self-assembly structures from a bead or other composition of the invention may be determined by contacting the composition with water and observing for such structures using a suitable analytical method such as dynamic light scattering.

The oil phase may also include one or more volatile or non-volatile solvents, which may be the same or different from the solvent or co-solvent previously mentioned. Such solvents may for example remain in the composition of the invention following processing e.g. initial dissolution of the components present in the core, and have no particular function in the core composition. Alternatively, such solvents if present may function to maintain the cyclosporin dissolved state (in solution) within the oil phase or to facilitate dispersion, egress etc. In other embodiments, the solvent may have partly or fully evaporated during processing and therefore be present in only minor quantities if at all. In a related embodiment, the solvent, particularly when a solvent which is both oil and water-soluble is used, may be partly or completely present in the aqueous phase of the core. An example of such a solvent is ethanol. Another example is Transcutol P or HP (2-(ethoxyethoxy)ethanol), which is already mentioned as a co-solvent.

Accordingly, the core may comprise a hydrogel-forming polymer matrix which forms a continuous phase and a disperse phase comprising cyclosporin, a high HLB non-ionic surfactant compound, a low HLB oil, and optionally a co-solvent.

The core may comprise a continuous phase which is or comprises a hydrogel-forming polymer and a disperse phase which is or comprises cyclosporin and an oil phase, the oil phase comprising an oil and one or more surfactants, wherein the oil and the surfactant have an HLB of up to 10. The presence of a surfactant with an HLB of up to 10 has been found to provide advantageous effects during the manufacture of the composition by for example inhibiting crystallisation of cyclosporin from the oil phase when the disperse phase is mixed with the continuous phase to form a colloid, for example an oil in water emulsion. Such compositions form a further aspect of the invention.

The presence of a surfactant with an HLB of up to 10 in the oil phase may enhance the rate and or extent of release of cyclosporin from the composition following oral administration. The presence of the surfactant may act to maintain a high proportion of the cyclosporin in a solubilised form after it has been released from the composition into an aqueous medium such as that found in the lower GI tract, particularly the colon.

Accordingly, the oral modified release composition may comprise a core having the form of a solid colloid, the colloid comprising a continuous phase being or comprising a hydrogel forming polymer and a disperse phase being or comprising cyclosporin, and an oil phase, the oil phase comprising an oil and one or more surfactants, wherein the surfactant has an HLB of up to 10, for example an HLB in the range 1-10.

The HLB value of the surfactant present in the oil phase may be may be up to 8, up to 7, 1-8, 1-7, 1-5, 2-5, 1-4, 1-3, 1-2, 2-4, 3-4, 5-8, 6-8 or 6-7, for example the HLB value may be about 1, about 2, about 3, about 4, about 5, about 6 or about 7. The surfactant may be any surfactant having an HLB value with the ranges described above, for example any of the surfactants described herein under the section “surfactants” herein or elsewhere in the description and examples. The surfactant is suitably a non-ionic surfactant. The cyclosporin may be soluble in the surfactant, for example the cyclosporin may have a solubility of more than about 200 mg/g in the surfactant. Thus, the surfactant may have a cyclosporin solubility of more than about 200 mg/g, optionally more than about 250 mg/g. The surfactant may have a cyclosporin solubility of from about 200 mg/g to about 500 mg/g, optionally from about 250 mg/g to about 500 mg/g, about 200 mg/g to about 400 mg/g, from about 225 mg/g to about 375 mg/g, from about 250 mg/g to about 375 mg/g, from about 200 mg/g to about 300 mg/g, from about 300 mg/g to about 400 mg/g, from about 250 mg/g to about 350 mg/g, from about 225 mg/g to about 275 mg/g, from about 350 mg/g to about 400 mg/g. Preferably, the surfactant has a cyclosporin solubility of from about 200 mg/g to about 400 mg/g or from about 225 mg/g to about 375 mg/g. Solubility of cyclosporin in a surfactant may be carried out following the protocol described in Development of a Self Micro-Emulsifying Tablet of Cyclosporin by the Liquisolid Compact Technique, Zhao et al (International Journal of Pharmaceutical Sciences and Research, 2011, Vol. 2(9), 2299-2308) which is incorporated herein by reference.

The surfactant may have an HLB of up to 6 and a cyclosporin solubility of from 200 mg/g to 400 mg/g. The surfactant may have an HLB value of 2-6 (optionally 3-6) and a cyclosporin solubility of from about 200 mg/g to about 400 mg/g. The surfactant may have an HLB value of 2-6 (optionally 3-6) and a cyclosporin solubility of from about 250 mg/g to about 400 mg/g. The surfactant may have an HLB value of 2-6 (optionally 3-6) and a cyclosporin solubility of from about 225 mg/g to about 275 mg/g. The surfactant may have an HLB value of 2-6 (optionally 3-6) and a cyclosporin solubility of from about 250 mg/g to about 350 mg/g.

The surfactant may be or comprise a surfactant selected from: fatty acid glycerides, polyethylene glycol fatty acid esters, propylene glycol fatty acid esters, fatty acid lactic acid ester, sucrose fatty acid esters, polyethylene glycol fatty alcohol ethers, ethylene oxide-propylene oxide block co-polymers and polyoxyethylene ethers; wherein the surfactant has an HLB value of up to 10, up to 8, or particularly a HLB value described above for example 1 to 8, or 1 to 4.

The surfactant may be or comprise a surfactant selected from: fatty acid glycerides, polyethylene glycol fatty acid esters, propylene glycol fatty acid esters, fatty acid lactic acid esters or sucrose fatty acid esters, wherein the surfactant has an HLB value of up to 10, up to 8, or particularly a HLB value described above for example 1 to 8 or 1 to 4.

The surfactant may be or comprise a fatty acid glyceride, wherein the surfactant has an HLB value of up to 10, up to 8, or particularly a HLB value described above, for example 1 to 8 or 1 to 4.

The surfactant may be or comprise a sorbitan fatty acid ester, for example a sorbitan mono, di- or tri-fatty acid ester and wherein the surfactant has an HLB value described above, for example 1 to 8 or 1 to 4. The fatty acid may be or comprise for example one or more C₁₀-C₂₀, C₁₂-C₂₀ or C₁₅-C₂₀ fatty acids more particularly a C₁₆ or C₁₈ fatty acid. The fatty acids may be saturated or unsaturated. A particular surfactant is or comprises sorbitan trioleate (commercially available as Span 85), Another particular surfactant is or comprises sorbitan monopalmitate (commercially available as Span 40).

The surfactant may be or comprise polyethylene glycol fatty acid esters, suitably esters with for example one or more C₁₀-C₂₀, C₁₂-C₂₀ or C₁₅-C₂₀ fatty acid, which acid may be saturated or unsaturated. Suitably the surfactant is or comprises a mixture comprising polyethylene glycol fatty acid esters and fatty acid glycerides, wherein the fatty acid is a C₁₅-C₂₀ fatty acid, which may be saturated or unsaturated. A particular surfactant is or comprises a mixture of oleoyl polyethylene glycol and oleoyl glycerides, for example oleoyl macrogol-6 glycerides (commercially available as Labrafil M1944CS).

The surfactant may be or comprise a polyglycerised fatty acid for example polyglyceryl dioleate. Accordingly the surfactant may act as an emulsifier and may be polyglyceryl-3 dioleate (for example products sold under the trade mark Plurol® Oleique).

The weight ratio of surfactant having a HLB value of up to 10:oil may be from about 5:1 to about 1:5, from about 3:1 to about 1:2, from about 3:1 to about 1:1 or from about 2.5:1 to 1.5:1. Suitably the weight ratio may be about 1:1, about 2:1, about 2.5:1, about 3:1, about 1:1.5 or about 1:2.

The surfactant having a HLB value of up to 10 may be present in the composition in an amount of from about 5% to about 20%, from about 8% to about 15%, or from about 10% to about 14% by weight based upon the dry weight of the core. It is to be understood that reference to the “dry weight of the core” means the weight of the components present in the uncoated core other than water.

The oil may be any of the oils described herein, particularly the oils described in the section “Disperse Phase”. The oil may be or comprise a short-, medium- or long-chain triglyceride composition, or a combination thereof. A medium chain triglyceride(s) (MCT) comprises one or more triglycerides of at least one fatty acid selected from C₆, C₇, C₈, C₉, C₁₀, C₁₁ and C₁₂ fatty acids. A particular oil phase is, or comprises a triglyceride based oil, such as those commercially available as Miglyol™, for example Miglyol™ 810, 812 (caprylic/capric triglyceride); Miglyol™ 818: (caprylic/capric/linoleic triglyceride); Miglyol™ 829: (caprylic/capric/succinic triglyceride).

The oil may be present in the composition in an amount of from about 2% to about 25%, from about 3% to about 20%, from about 3% to about 10% or from about 5% to about 10% by weight based upon the dry weight of the core.

The oil phase may also comprise a solvent. Suitable solvents are as described herein in relation to the disperse phase and are suitable miscible with both the oil and water. The solvent may be presently in the composition in an amount of form about 1% to 30%, for about 5% to about 30%, for about 10% to about 25%, or from about 12% to about 22% by weight based upon the dry weight of the core. A particular solvent is 2-(2-ethoxyethoxy)ethanol (available commercially as for example Transcutol™ P or HP).

The hydrogel-forming polymer may be or comprise one or more of the hydrogel-forming polymers described herein, particularly those described under “Continuous Phase Polymer Matrix”. Suitably the hydrogel-forming polymer is or comprises a hydrogel-forming polymer selected from the group consisting of gelatin, agar, a polyethylene glycol, starch, casein, chitosan, soya bean protein, safflower protein, alginates, gellan gum, carrageenan, xanthan gum, phthalated gelatin, succinated gelatin, cellulosephthalate-acetate, oleoresin, polyvinylacetate, hydroxypropyl methyl cellulose, polymerisates of acrylic or methacrylic esters and polyvinylacetate-phthalate and any derivative of any of the foregoing; or a mixture of one or more such a hydrogel forming polymers. A particular hydrogel-forming polymer is selected from carrageenan, gelatin, agar and pectin, or a combination thereof, particularly gelatin and/or agar, more particularly gelatin. The hydrogel forming polymer is suitably present in the core in a gelled state such that the polymer forms a solid matrix within which the disperse phase is dispersed to provide for example a solid colloid. The hydrogel-forming polymer is preferably sufficiently gelled to provide a core which is sufficiently rigid to enable to be handled and further processed into a dosage form or to be coated with for example a modified release coating as described herein.

The hydrogel-forming polymer may be present in an amount of from about 20% to about 70%, about 20% to about 55%, about 25% to about 50%, about 30% to about 50%, or about 40% to about 45% by weight based upon the dry weight of the core.

The continuous phase may comprise a suitably plasticiser, particularly when the hydrogel-forming polymer is or comprises gelatin. A particular plasticiser is Sorbitol. When present the plasticiser may be present at for example up to about 20% or up to about 10%, suitably from about 3% to about 8%, or from about 4% to about 6% by weight based upon the dry weight of the core.

The continuous phase may comprise a surfactant. The surfactant present in the continuous phase is preferably different to the surfactant present in the oil phase. Suitable surfactants which may be present in the continuous phase are as described herein under the section “Continuous Phase Polymer Matrix”. Accordingly particular surfactants which may be present in the continuous phase may be cationic, amphoteric (zwitterionic) or anionic surfactants. Suitably the surfactant present in the continuous phase is or comprises an anionic surfactant, more particularly a hydrophilic anionic surfactant. The surfactant in the continuous phase may be or comprise at least one surfactant selected from fatty acid salts, alkyl sulfates and bile salts, particularly an alkyl sulfate, for example a C₁₀-C₂₂ alkyl sulphate suitably sodium dodecyl sulphate. The surfactant present in the continuous phase, particularly anionic surfactant is present in the composition in an amount of from 0.1% to 6%, e.g. 0.1% to 5%. 0.1% to 4%, 0.1% to 3%, 1% to 4%, 1.5% to 4.5%, or 2.5% to 4.5% preferably in an amount 2-4% by weight based upon the dry weight of the core.

The cyclosporin is suitably present in the composition in an amount for from about 5% to about 20%, from about 8% to about 15%, or from about 9% to about 14% % by weight based upon the dry weight of the core.

In a particular embodiment the oral modified release composition comprises a core having the form of a solid colloid, the colloid comprising a continuous phase being or comprising a hydrogel forming polymer and a disperse phase;

wherein the disperse phase is or comprises:
cyclosporin;
an oil being or comprising: a short-, medium- or long-chain triglyceride composition, or a combination thereof, for example a caprylic/capric triglyceride, a caprylic/capric/linoleic triglyceride; and a caprylic/capric/succinic triglyceride;
one or more non-ionic surfactants with an value HLB of up to 10, up to 8, up to 7, 1-8, 1-7, 1-5, 2-5, 1-4, 1-3, 1-2, 2-4, 3-4, 5-8, 6-8 or 6-7, for example about 1, about 2, about 3, about 4, about 5, about 6 or about 7; optionally wherein the surfactant is or comprises a fatty acid glyceride, a sorbitan fatty acid ester, or a polyethylene glycol fatty acid ester; and
optionally a solvent, wherein the solvent is miscible with the oil and with water, for example 2-(2-ethoxyethoxy)ethanol;
   wherein the continuous phase is or comprises:
   a hydrogel-forming polymer, for example a hydrogel forming polymer being or comprising carrageenan, gelatin, agar and pectin, or a combination thereof, optionally gelatin or agar or a combination thereof, more optionally the polymer of the a hydrogel forming polymer matrix is or comprises gelatin;
   an anionic surfactant, optionally an anionic surfactant is selected from fatty acid salts, alkyl sulphates and bile salts, particularly an alkyl sulfate, for example a C₁₀-C₂₂ alkyl sulphate suitably, sodium dodecyl sulphate; and optionally a plasticiser, for example sorbitol.

In another embodiment the oral modified release composition comprises a core having the form of a solid colloid, the colloid comprising a continuous phase being or comprising a hydrogel forming polymer and a disperse phase;

wherein the disperse phase is or comprises:
from about 8% to about 15% cyclosporin;
from about 2% to about 20%, for example about 3% to about 10% of oil being or comprising a caprylic/capric triglyceride, a caprylic/capric/linoleic triglyceride; and a caprylic/capric/succinic triglyceride, preferably a caprylic/capric triglyceride;
one or more non-ionic surfactants with an value HLB of up to 10, up to 8, up to 7, 1-8, 1-7, 1-5, 2-5, 1-4, 1-3, 1-2, 2-4, 3-4, 5-8, 6-8 or 6-7, for example about 1, about 2, about 3, about 4, about 5, about 6 or about 7; optionally wherein the surfactant is or comprises a fatty acid glyceride, a sorbitan fatty acid ester, or a polyethylene glycol fatty acid ester, optionally wherein the non-ionic surfactant is present in an amount of from about 8% to about 15%; and
optionally from about 12% to about 22% solvent, wherein the solvent is miscible with the oil and with water, for example 2-(2-ethoxyethoxy)ethanol;
   wherein the continuous phase is or comprises:
from about 30% to about 70%, for example about 30% to about 50% hydrogel-forming polymer, optionally wherein the hydrogel forming polymer is or comprises carrageenan, gelatin, agar and pectin, or a combination thereof, optionally gelatin or agar or a combination thereof, more optionally wherein the hydrogel forming polymer matrix is or comprises gelatin;
an anionic surfactant, optionally an anionic surfactant is selected from fatty acid salts, alkyl sulphates and bile salts, particularly an alkyl sulfate, for example a C₁₀-C₂₂ alkyl sulphate suitably sodium dodecyl sulphate, optionally wherein the anionic surfactant is present in an amount of from about 0.1% to about 5%, suitably from 2% to 4%; and
optionally up to about 10% plasticiser, for example sorbitol;
   wherein all % are % by weight based upon the dry weight of the core.

In another embodiment the an oral modified release composition comprises a core having the form of a solid colloid, the colloid comprising a continuous phase being or comprising a hydrogel forming polymer and a disperse phase;

wherein the disperse phase is or comprises:
from about 8% to about 15% cyclosporin;
from about 3% to about 10% of oil being or comprising a caprylic/capric triglyceride;
one or more non-ionic surfactants with an value HLB of up to 7, for example 1-7, or 2-4 wherein the surfactant is or comprises a fatty acid glyceride, a sorbitan fatty acid ester, or a polyethylene glycol fatty acid ester, optionally wherein the non-ionic surfactant is present in an amount of from about 8% to about 15%; and
optionally from about 12% to about 22% solvent, wherein the solvent is miscible with the oil and with water, for example 2-(2-ethoxyethoxy)ethanol;
   wherein the continuous phase is or comprises:
from about 30% to about 50% hydrogel-forming polymer selected from gelatin or agar or a combination thereof, optionally wherein the hydrogel forming polymer matrix is or comprises gelatin;
0.1% to about 5%, suitably from 2% to 4% anionic surfactant for example sodium dodecyl sulphate; and
optionally up to about 10% plasticiser, for example sorbitol;
   wherein all % are % by weight based upon the dry weight of the core.

In another particular embodiment the core is in the form of a solid colloid, the colloid comprising a continuous phase and a disperse phase, wherein the continuous phase comprises the hydrogel forming polymer; wherein

the disperse phase is or comprises:
cyclosporin;
a medium chain mono-, di- or tri-glyceride, for example a medium chain triglyceride, particularly caprylic/capric triglyceride;
a non-ionic surfactant (for example a polyethoxylated castor oil); and
a solvent (for example 2-(ethoxyethoxy)ethanol);
   and wherein the continuous phase is or comprises:
a hydrogel forming polymer matrix which is or comprises a hydrocolloid selected from carrageenan, gelatin, agar and pectin, or a combination thereof optionally selected from gelatin and agar or a combination thereof, more optionally the polymer of the a hydrogel forming polymer matrix is or comprises gelatin;
optionally a plasticiser, for example a plasticiser selected from glycerin, a polyol for example sorbitol, polyethylene glycol and triethyl citrate or a mixture thereof, particularly sorbitol; and
an anionic surfactant, for example at least one surfactant selected from fatty acid salts, alkyl sulphates and bile salts, particularly an alkyl sulfate, for example sodium dodecyl sulfate.

In a further specific embodiment the core comprises a hydrogel forming polymer matrix comprising gelatin in an amount of 300 to 700 mg/g, the core further comprising cyclosporin, medium chain mono-, di- or tri-glycerides (for example a medium chain triglyceride, particularly caprylic/capric triglyceride) in an amount of 20 to 200 mg/g, and the core further comprises the following components:
solvent (for example 2-(ethoxyethoxy)ethanol) in an amount of 150 to 250 mg/g;
non-ionic surfactant in an amount of 80 to 200 mg/g; and
anionic surfactant in an amount of 15 to 50 mg/g,
   wherein weights are based upon the dry weight of the core.

Suitably in the embodiment of the above paragraph the cyclosporin may be present in an amount of 60 to 150 mg/g, for example 80 to 120 mg/g or particularly 80 to 100 mg/g. The non-ionic and anionic surfactants are as defined herein, for example an anionic surfactant selected from alkyl sulfates, carboxylates or phospholipids (particularly SDS); or a non-ionic surfactant selected from sorbitan-based surfactants, PEG-fatty acids, or glyceryl fatty acids or poloxamers. A particular non-ionic surfactant is a polyethoxylated castor oil (for example Kolliphor™ EL).

The cores described above comprising hydrogel-forming polymer matrix and cyclosporin are coated as described herein to provide a modified release composition according to the invention. A particular coating for these embodiments is a coating comprising a first coating (sub-coating) which is or comprises a water-soluble cellulose ether, particularly hydroxypropylmethyl cellulose;

a second coating outside the first coating which is or comprises a modified release coating, particularly a pH independent modified release coating, more especially a coating comprising ethyl cellulose (e.g. Surelease™) still more particularly a coating comprising ethyl cellulose and a water-soluble polysaccharide such as pectin (e.g. a Surelease™ pectin coating as described herein); and wherein

the first coating is present in an amount corresponding to a weight gain due to the first coating in a range selected from: (i) from 1% to 20%; (ii) from 8% to 12%, for example about 10%; or (iii) from 4% to 6%, for example about 5% by weight based upon the weight of the composition prior to applying the first coating; and wherein

the second coating is present in an amount corresponding to a weight gain of the composition due to the second coating selected from (a) from 5 to 40%; (b) from 10% to 12%, for example about 11% or about 11.5%; or (c) from 16% to 18%, for example about 17% by weight based upon the weight of the composition prior to applying the second coating.

The compositions described herein are optionally further coated with a suitable protective coating as described herein. For example the protective coating may comprise a water-soluble cellulosic or PVA film-forming polymer such as an Opadry™ coating. The protective coating is suitably applied to the outer surface of the composition as described above.

### Surfactant

The composition may contain one or more surfactant, for example surfactants may be present in the core (including in the hydrogel-forming polymer matrix, and in the disperse phase or both). Surfactants may also be present in one or more of the coatings applied to the core.

Suitable surfactants can be anionic, cationic, zwitterionic, or non-ionic. In the description and claims of this specification, the term “surfactant” is employed as a contraction for “surface active agent”. For the purposes of this description and claims, it is assumed that there are four major classifications of surfactants; therefore the surfactant may be: anionic, cationic, non-ionic, and amphoteric (zwitterionic). The non-ionic surfactant remains whole, has no charge in aqueous solutions, and does not dissociate into positive and negative ions. Anionic surfactants are water-soluble, have a negative charge and dissociate into positive and negative ions when placed in water. The negative charge lowers the surface tension of water and acts as the surface-active agent. Cationic surfactants have a positive charge, and also dissociate into positive and negative ions when placed in water. In this case, the positive ions lower the surface tension of the water and act as the surfactant. The amphoteric (zwitterionic) surfactant assumes a positive charge in acidic solutions and performs as a cationic surfactant, or it assumes a negative charge in an alkaline solution and acts as an anionic surfactant.

The surfactant(s) may be selected from: anionic surfactants and combinations thereof; from non-ionic surfactants and combinations thereof; and from combination of an anionic surfactant (e.g. a single such surfactant or a plurality thereof) and a non-ionic surfactant (e.g. a single such surfactant or a plurality thereof).

Surfactants can also be classified according to their hydrophilic-lipophilic balance (HLB) which is a measure of the degree to which the surfactant is hydrophilic or lipophilic, determined by calculating values for the different regions of the molecule, as described (originally for non-ionic surfactants) by Griffin in 1949 and 1954 and later by Davies. The methods apply a formula to the molecular weight of the whole molecule and of the hydrophilic and lipophilic portions to give an arbitrary (semi-empirical) scale up to 40 although the usual range is between 0 and 20. An HLB value of 0 corresponds to a completely hydrophobic molecule, and a value of 20 would correspond to a molecule made up completely of hydrophilic components. The HLB value can be used to predict the surfactant properties of a molecule:

| HLB Value | Expected properties |
|---|---|
| 0 to 3 | antifoaming agent |
| from 4 to 6 | W/O emulsifier |
| from 7 to 9 | wetting agent |
| from 8 to 18 | an O/W emulsifier |
| from 13 to 15 | typical of detergents |
| 10 to 18 | solubiliser or hydrotrope |

Although HLB numbers are assigned to surfactants other than the non-ionic, for which the system was invented, HLB numbers for anionic, cationic, non-ionic, and amphoteric (zwitterionic) surfactants can have less significance and often represent a relative or comparative number and not the result of a mathematical calculation. This is why it is possible to have surfactants above the “maximum” of 20. HLB numbers can however be useful to describe the HLB requirement of a desired application for a given emulsion system in order to achieve good performance.

### Non-Ionic Surfactants

The surfactant may be or comprise at least one surfactant selected from the following non-ionic surfactants.

PEG-fatty acid monoester surfactants, PEG-fatty acid diester surfactants, PEG-fatty acid monoester and diester surfactant mixtures, PEG glycerol fatty acid esters, transesterified products of oils and alcohols, lower alcohol fatty acid esters, polyglycerised fatty acids, propylene glycol fatty acid esters, mono and diglyceride surfactants, sterol and sterol derivative surfactants, PEG-sorbitan fatty acid esters, sorbitan fatty acid esters, polyethylene glycol alkyl ethers, sugar ester surfactants, polyethylene glycol alkyl phenol surfactants, POE-POP block copolymers, fatty acid salts, bile salts, phospholipids, phosphoric acid esters, carboxylates, acyl lactylates, sulfates and sulfonates, and cationic surfactants.

A PEG-fatty acid mono ester surfactant for example PEG 4-100 monolaurate, PEG 4-100 monooleate, PEG 4-100 monostearate, PEG-laurate, PEG-oleate, PEG stearate, and PEG ricinoleate. A PEG-fatty acid diester surfactant for example PEG dilaurate; PEG dioleate, PEG distearate, PEG dipalmitate. A mixture of PEG-fatty acid mono- and diesters.

A PEG glycerol fatty acid ester for example PEG glyceryl laurate, PEG glyceryl stearate, PEG glyceryl oleate.

PEG-sorbitan fatty acid esters for example PEG sorbitan laurate, PEG sorbitan monolaurate, PEG sorbitan monopalmitate, PEG sorbitan monostearate, PEG sorbitan tristearate, PEG sorbitan tetrastearate, PEG sorbitan monooleate, PEG sorbitan oleate, PEG sorbitan trioleate, PEG sorbitan tetraoleate, PEG sorbitan monoisostearate, PEG sorbitol hexaoleate, PEG sorbitol hexastearate.

Propylene glycol fatty acid esters for example propylene glycol monocaprylate, propylene glycol monolaurate, propylene glycol oleate, propylene glycol myristate, propylene glycol monostearate, propylene glycol hydroxy stearate, propylene glycol ricinoleate, propylene glycol isostearate, propylene glycol monooleate, propylene glycol dicaprylate/dicaprate, propylene glycol dioctanoate, propylene glycon caprylate/caprate, propylene glycol dilaurate, propylene glycol distearate, propylene glycol dicaprylate, propylene glycol dicaprate.

A sorbitan fatty acid ester for example sorbitan monolaurate, sorbitan monopalmitate, sorbitan monooleate, sorbitan monostearate, sorbitan trioleate, sorbitan sesquioleate, sorbitan tristearate, sorbitan monoisostearate, sorbitan sesquistearate.

Lower alcohol fatty acid esters for example ethyl oleate, isopropy myristate, isopropyl palmitate, ethyl linoleate, isopropyl linoleate.

Polyoxyethylene-polyoxypropylene block copolymers for example poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407.

Polyglycerised fatty acids for example polyglyceryl stearate, polyglyceryl oleate, polyglyceryl isostearate, polyglyceryl laurate, polyglyceryl ricinoleate, polyglyceryl linoleate, polyglyceryl pentaoleate, polyglyceryl dioleate, polyglyceryl distearate, polyglyceryl trioleate, polyglyceryl septaoleate, polyglyceryl tetraoleate, polyglyceryl decaisostearate, polyglyceryl decaoleate, polyglyceryl monooleate, dioleate, polyglyceryl polyricinoleate.

PEG alkyl ethers for example PEG oleyl ether, PEG lauryl ether, PEG cetyl ether, PEG stearyl ether.

PEG alkyl phenols for example PEG nonyl phenol, PEG octyl phenol ether.

Transesterification products of alcohol or polyalcohol with natural or hydrogenated oils for example PEG castor oil, PEG hydrogenated castor oil, PEG corn oil, PEG almond oil, PEG apricot kernel oil, PEG olive oil, PEG-6 peanut oil, PEG hydrogenated palm kernel oil, PEG palm kernel oil, PEG triolein, PEG corn glycerides, PEG almond glycerides, PEG trioleate, PEG caprylic/capric triglyceride, lauroyl macrogol glyceride, stearoyl macrogol glyceride, mono, di, tri, tetra esters of vegetable oils and sorbitol, pentaerythrityl tetraisostearate, pentaerythrityl distearate, pentaerythrityl tetraoleate, pentaerythrityl tetrastearate, pentaerythrityl tetracaprylate/tetracaprate, pentaerythrityl tetraoctanoate.

Oil-soluble vitamins for example vitamins A, D, E, K, and isomers, analogues, and derivatives thereof. The derivatives include, for example, organic acid esters of these oil-soluble vitamin substances, for example the esters of vitamin E or vitamin A with succinic acid. Derivatives of these vitamins include tocopheryl PEG-1000 succinate (Vitamin E TPGS) and other tocopheryl PEG succinate derivatives with various molecular weights of the PEG moiety, for example PEG 100-8000.

Sterols or sterol derivatives (e.g. esterified or etherified sterols as for example PEGylated sterols) for example cholesterol, sitosterol, lanosterol, PEG cholesterol ether, PEG cholestanol, phytosterol, PEG phytosterol.

Sugar esters for example sucrose distearate, sucrose distearate/monostearate, sucrose dipalmitate, sucrose monostearate, sucrose monopalmitate, sucrose monolaurate, alkyl glucoside, alkyl maltoside, alkyl maltotrioside, alkyl glycosides, derivatives and other sugar types: glucamides.

Carboxylates (in particular carboxylate esters) for example ether carboxylates, succinylated monoglycerides, sodium stearyl fumarate, stearoyl propylene glycol hydrogen succinated, mono/diacetylated tartaric acid esters of mono- and diglycerides, citric acid esters of mono-, diglycerides, glyceryl-lacto esters of fatty acids; acyl lactylates: lactylic esters of fatty acids, calcium/sodium stearoyl-2-lactylate calcium/sodium stearoyl lactylate, alginate salts, propylene glycol alginate.

A fatty acid monoglyceride, diglyceride or triglyceride or a combination thereof.

### Anionic Surfactants

Anionic surfactants may be selected from following anionic surfactants.

Fatty acid salts and bile salts for example sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate, sodium tetradecyl sulfate, sodium lauryl sarcosinate, sodium dioctyl sulfosuccinate; sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco chenodeoxycholate, sodium cholylsarcosinate, sodium N-methyl taurocholate

Phospholipids for example egg/soy lecithin, cardiolipin, sphingomyelin, phosphatidylcholine, phosphatidyl ethanolamine, phosphatidic acid, phosphatidyl glycerol, phosphatidyl serine.

Phosphoric acid esters having the general formula RO—PO₃⁻M⁺ where the R group is an ester forming group, e.g. an alkyl, alkenyl or aryl group optionally substituted by a PEG moiety through which the alkyl, alkenyl or aryl group is coupled to the phosphate moiety. R may be a residue of a long chain (e.g. >C₉) alcohol or a phenol. Specific examples include diethanolammonium polyoxyethylene-10 oleyl ether phosphate, esterification products of fatty alcohols or fatty alcohol ethoxylates with phosphoric acid or anhydride.

Sulfates and sulfonates (in particular esters thereof) for example ethoxylated alkyl sulfates, alkyl benzene sulfones, α-olefin sulfonates, acyl isethionates, acyl taurates, alkyl glyceryl ether sulfonates, octyl sulfosuccinate disodium, disodium undecylenamideo-MEA-sulfosuccinate, alkyl phosphates and alkyl ether phosphates.

Particular anionic surfactants include alkyl sulfates, for example. C₁₀-C₂₂ alkyl sulfates such as sodium dodecyl sulfate.

The anionic surfactant may be perfluoro-octanoate (PFOA or PFO), perfluoro-octanesulfonate (PFOS), sodium dodecyl sulfate (SDS), ammonium lauryl sulfate, and other alkyl sulfate salts, sodium laureth sulfate, also known as sodium lauryl ether sulfate (SLES) and alkyl benzene sulphonate. A particular class of surfactant comprises alkyl sulfate salts. A preferred anionic surfactant is SDS.

### Cationic Surfactants

Cationic surfactants may be selected from the following cationic surfactants.

Hexadecyl triammonium bromide, dodecyl ammonium chloride, alkyl benzyldimethylammonium salts, diisobutyl phenoxyethoxydimethyl benzylammonium salts, alkylpyridinium salts; betains (trialkylglycine): lauryl betaine (N-lauryl,N,N-dimethylglycine); ethoxylated amines: polyoxyethylene-15 coconut amine, alkyl-amines/diamines/quaternary amines and alkyl ester.

### Emulsifiers

The surfactant may act as an emulsifier such surfactants include non-ionic emulsifiers, for example selected from: a mixture of triceteareth-4 phosphate, ethylene glycol palmitostearate and diethylene glycol palmitostearate (for example sold under the trade mark SEDFOS™ 75); sorbitan esters, e.g. sorbitan monooleate, sorbitan monolaurate, sorbitan monpalmitate, sorbitan monostearate (for example products sold under the trade mark Span™), PEG-8 beeswax e.g. sold under the trade mark Apifil®; a mixture of cetyl alcohol, ceteth-20 and steareth-20 (for example Emulcire™ 61 WL 2659); a mixture of glyceryl monostearate EP/NF and PEG-75 palmitostearate (for example Gelto™ 64); a mixture of PEG-6 stearate and PEG-32 stearate (for example Tefose® 1500); a mixture of PEG-6 palmitostearate, ethylene glycol palmitostearate, and PEG-32 palmitostearate (e.g. Tefose® 63); triglycerol diisostearate (for example products sold under the trade mark Plurol Diisosteariquel; polyglyceryl-3 dioleate (for example products sold under the trade mark Plurol® Oleique).

### Other Excipients

The modified release composition optionally contains one or more of the following additional substances or categories of substances. For example, the composition may contain a protectant such as, for example, a proteolytic enzyme inhibitor or a protector against acid degradation or both (e.g. an alkali for example sodium hydroxide); an adhesive entity such as, for example, a muco- or bio-adhesive; excipients to maximize solubility of the cyclosporin; excipients to maximize permeability of the cyclosporin in the GIT. Typical excipients for enhancing the permeability of the epithelial barrier include but are not limited to sodium caprate, sodium dodecanoate, sodium palmitate, SNAC, chitosan and derivatives thereof, fatty acids, fatty acid esters, polyethers, bile salts, phospholipids, alkyl polyglucosides, antioxidants (e.g. ascorbic acid) and/or nitric oxide donors. The preceding list is of particular interest to enhance permeability in the ileum.

To enhance permeability in the colon, typical excipients include, but not limited to sodium caprate, sodium dodecanoate, sodium palmitate, SNAC, chitosan and derivatives thereof, fatty acids, fatty acid esters, polyethers, bile salts, phospholipids, alkyl polyglucosides, antioxidants and/or nitric oxide donors, including nitric oxide donor groups covalently attached to various active pharmaceutical ingredients.

The composition may further comprise excipients to enhance the therapeutic potential of the cyclosporin in the ileum and colon including, but not limited to absorption limiters, essential oils such as, for example, omega 3 oils, natural plant extracts such as, for example, neem, ion-exchange resins, bacteria degradable conjugation linkers such as, for example, azo bonds, polysaccharides such as, for example, amylose, guar gum, pectin, chitosan, inulin, cyclodextrins, chondroitin sulphate, dextrans, guar gum and locust bean gum, nuclear factor kappa B inhibitors, acids such as, for example, fumaric acid, citric acid and others, as well as modifications thereof.

The composition may further comprise excipients to reduce systemic side effects associated with absorption in the GIT, such as the small intestine, including, but not limited to, antioxidants, such as, for example, curcuminoids, flavanoids or more specifically including curcumin, beta-carotene, α-tocopherol, ascorbate or lazaroid.

The composition may further or separately comprise antioxidants (such as, for example, ascorbic acid or BHT—butyl hydroxy toluene) taste-masking or photosensitive components or photoprotective components. Antioxidants may be incorporated in the aqueous phase (e.g. hydrophilic antioxidants) or in the disperse phase of the core (e.g. hydrophobic antioxidants such as, for example, vitamin E) for example up to 1% by weight, preferably between 0.01 and 0.50% by weight, more preferably between 0.10 to 0.20% by weight.

The composition may further comprise immune-enhancing nutrients such as vitamins A/B/C/E; carotenoids/beta-carotene and iron, manganese, selenium or zinc. Such nutrients may be present in composition, or if the composition has a coating, for example if it is the form of a bead, the nutrients may be included in the coating.

The composition may also include other well know excipients used in pharmaceutical compositions including colorants, taste masking agents, diluents, fillers, binders etc. The presence of such optional additional components will of course depend upon the particular dosage form adopted.

### Shape, Size and Geometry

The composition of the invention can be formed into a limitless number of shapes and sizes. In the section below describing the process for making the composition, various methods are given including pouring or introducing a fluid dispersion into a mould where it hardens or can be caused to harden. Thus the composition can be created in whichever form is desired by creating an appropriate mould (e.g. in the shape of a disc, pill or tablet). However, it is not essential to use a mould. For example, the composition may be formed into a sheet e.g. resulting from pouring a fluid dispersion onto a flat surface where it hardens or can be caused to harden.

Preferably, the composition may be in the form of spheres or spherical-like shapes made as described below. Preferably, the composition of the invention is in the form of substantially spherical, seamless minibeads. The absence of seams on the minibead surface is an advantage e.g. in further processing, for example coating, since it allows more consistent coating, flowability etc. The absence of seams on the minibeads also enhances consistency of dissolution of the beads.

The preferred size or diameter range of minibeads according to the invention can be chosen to avoid retention in the stomach upon oral administration of the minibeads. Larger dosage forms are retained for variable periods in the stomach and pass the pyloric sphincter only with food whereas smaller particles pass the pylorus independently of food. Selection of the appropriate size range (see below) thus makes the therapeutic effect post-dosing more consistent. Compared to a single large monolithic oral format such as, for example, a traditional compressed pill, a population of beads released into the GI tract (as foreseen by the dosage form of the present invention) permits greater intestinal lumen dispersion so enhancing absorption via exposure to greater epithelial area, and achieves greater topical coating in certain parts of the GI tract for example the colon). Reduction of residence time in the ileo-caecal junction is another potential advantage.

The composition of the invention is preferably monolithic meaning internally (i.e. cross-sectionally) homogeneous, excluding a possible thin skin of matrix material and excluding any coating layers.

The minibeads provided for by the composition of the present invention generally range in diameter from 0.5 mm to 10 mm with the upper limit preferably 5 mm, e.g. 2.5 mm A particularly convenient upper limit is 2 mm or 1.7 mm. The lower limit can preferably be 1 mm, e.g. 1.2 mm, more preferably from 1.3 mm, most preferably from 1.4 mm. In one embodiment the diameter is from 0.5 to 2.5 mm, for example from 1 mm to 3 mm, 1 mm to 2 mm, 1.2 mm to 3 mm or 1.2 mm to 2 mm. The minibeads may have a diameter of no more than 2.5 mm, irrespective of their minimum size. The beads may have a diameter of no more than 2 mm, irrespective of their minimum size.

A minibead as described herein may have an aspect ratio of no more than 1.5, e.g. of no more than 1.3, for example of no more than 1.2 and, in particular, of from 1.1 to 1.5, 1.1 to 1.3 or, 1.1 to 1.2. A population of minibeads as described herein, e.g. at least 10 beads, may have an average aspect ratio of no more than 1.5, e.g. of no more than 1.3, for example of no more than 1.2 and, in particular, of from 1 to 1.5, 1 to 1.3 or 1 to 1.2. The aspect ratios mentioned in this paragraph optionally apply to coated minibeads and optionally apply to uncoated minibeads. Average aspect ratio is suitably determined for a population of minibeads, e.g. at least 10 minibeads, using a particle size analyser, for example an Eyecon™ particle characteriser of Innopharma Labs, Dublin 18, Ireland.

The minibeads of the disclosure may, therefore, have a size as disclosed above and an aspect ratio of from 1 to 1.5. The beads of the disclosure may have a size as disclosed above and an aspect ratio of no more than 1.3, for example of no more than 1.2 and, in particular, of from 1.1 to 1.5, 1.1 to 1.3 or, 1.1 to 1.2.

Bead size (diameter) may be measured by any suitable technique, for example microscopy, sieving, sedimentation, optical sensing zone method, electrical sensing zone method or laser light scattering. For the purposes of this specification, bead size is measured by analytical sieving in accordance with USP General Test <786> Method I (USP 24-NF 18, (U.S. Pharmacopeial Convention, Rockville, Md., 2000), pp. 1965-1967).

In embodiments, minibeads of the invention are monodisperse. In other embodiments, minibeads of the invention are not monodisperse. By “monodisperse” is meant that for a population of beads (e. g. at least 100, more preferably at least 1000) the minibeads have a coefficient of variation (CV) of their diameters of 35% or less, optionally 25% or less, for example 15% or less, such as e.g. of 10% or less and optionally of 8% or less, e.g. 5% or less. A particular class of polymer beads has a CV of 25% or less. CV when referred to in this specification is defined as 100 times (standard deviation) divided by average where “average” is mean particle diameter and standard deviation is standard deviation in particle size. Such a determination of CV is performable using a sieve.

The invention includes minibeads having a CV of 35% and a mean diameter of 1 mm to 2 mm, e.g. 1.5 mm. The invention also includes minibeads having a CV of 20% and a mean diameter of 1 mm to 2 mm, e.g. 1.5 mm, as well as minibeads having a CV of 10% and a mean diameter of 1 mm to 2 mm, e.g. 1.5 mm. In one class of embodiments, 90% of minibeads have a diameter of from 0.5 mm to 2.5 mm, e.g. of from 1 mm to 2 mm.

### Dosage Forms

The modified release composition of the invention is prepared as an orally administrable dosage form suitable for pharmaceutical use. In those embodiments where the composition is in the form of a minibead, the present invention provides for a dosage form comprising a plurality of the minibeads for example as a capsule, a tablet, a sprinkle or a sachet.

In embodiments the dosage form comprising a population of beads may be presented in a single unit dosage form e.g. contained in a single hard gel or HPMC capsule which releases the beads e.g. in the stomach. Alternatively the beads may be presented in a sachet or other container which permits the beads to be sprinkled onto food or into a drink or to be administered via a feeding tube for example a naso-gastric tube or a duodenal feeding tube. Alternatively, the beads may be administered as a tablet for example if a population of beads is compressed into a single tablet as described below. Alternatively, the beads may be filled e.g. compressed into a specialist bottle cap or otherwise fill a space in a specialised bottle cap or other element of a sealed container (or container to be sealed) such that e.g. on twisting the bottle cap, the beads are released into a fluid or other contents of the bottle or vial such that the beads are dispersed (or dissolve) with or without agitation in such contents. An example is the Smart Delivery Cap manufactured by Humana Pharma International (HPI) S.p.A, Milan, Italy.

The dosage form may be formulated in such a way so that the beads of the invention can be further developed to create a larger mass of beads e.g. via compression (with appropriate oil or powder-based binder and/or filler known to persons skilled in the art. The larger (e.g. compressed) mass may itself take a variety of shapes including pill shapes, tablet shapes, capsule shapes etc. A particular problem which this version of the bead embodiment solves is the “dead space” (above the settled particulate contents) and/or “void space” (between the particulate content elements) typically found in hard gel capsules filled with powders or pellets. In such pellet- or powder-filled capsules with dead/void space, a patient is required to swallow a larger capsule than would be necessary if the capsules contained no such dead space. The beads of this embodiment of the invention may readily be compressed into a capsule to adopt the inner form of whichever capsule or shell may be desired leaving much reduced, e.g. essentially no, dead/void space. Alternatively the dead or void space can be used to advantage by suspending beads in a vehicle such as, for example, an oil which may be inert or may have functional properties such as, for example, permeability enhancement or enhanced dissolution or may comprise an active ingredient being the same or different from any active ingredients in the bead. For example, hard gelatin capsules may be filled with a liquid medium combined with uncoated and/or coated beads. The liquid medium may be one or more of the surfactant phase constituents described herein or it may be one or more surfactants. Particularly preferred but non-limiting examples are corn oil, sorbitane trioleate (sold under the trade mark SPAN 85), propylene glycol dicaprylocaprate (sold under the trade mark Labrafac), 2-(2-ethoxyethoxy)ethanol (sold under the trade mark Transcutol P or HP) and polysorbate 80 (sold under the trade mark Tween 80).

In a representative embodiment the bead of the dosage form is prepared as described herein for example by mixing together at least the following materials: a hydrogel-forming polymer; and cyclosporin, suitably cyclosporin dissolved in a hydrophobic material, such as an oil to form a dispersion of the cyclosporin in the hydrogel-forming polymer. The dispersion is immobilized within the solidified bead by ejection from a single orifice nozzle into a suitable cooling liquid. Following removal of the drying liquid the bead is coated with a modified release coating (suitably with a sub-coat under the modified release coating), the coated bead is then formulated into the desired dosage form, for example filled into a capsule or sachet suitable for pharmaceutical use, for example a hard-gel, gelatin or HPMC capsule. Alternatively the beads may be formulated into a tablet composition together with suitable tablet excipients such as binders, fillers, and lubricants. The tablets may be prepared using conventional methods such as blending, granulation and/or and direct compression. The final dosage form may be packaged in a suitable format for use, for example in a blister pack or other suitable container such as a bottle containing tablets or capsules. Where the final dosage form is a sachet, the beads may be packaged in a suitable sachet material which provides a barrier to protect the beads contained in the sachet from, for example moisture and light and to maintain sterility prior to use. Such materials are well known and include laminates, for example paper laminates, foil laminates, polymeric films. The sachet material is optionally a gas permeable material, for example comprising a flashspun polyethylene fibre matrix, commercially available as Tyvek™.

Suitably the dosage form is prepared as a unit dosage form containing from for oral administration comprising from 0.1 mg to 1000 mg, optionally from 1 mg to 500 mg, for example 10 mg to 300 mg, or 25 to 250 mg, suitably about 25 mg, about 35 mg, about 37.5 mg, about 50 mg, about 75 mg, about 100 mg, about 105 mg, about 112.5 mg, about 125 mg, about 150 mg, 175 mg, about 180 mg, about 187.5 mg, about 200 mg, about 210 mg or about 250 mg cyclosporin.

### Manufacturing Processes

Various methods may be used to prepare the modified release compositions of the invention.

In those embodiments where the modified release composition comprises cyclosporin in a water-insoluble polymer matrix a basic method for making the composition is to mix a fluid form of the matrix material, for example a water-insoluble polymer matrix material (e.g. poly(amides), poly(amino-acids), hyaluronic acid; lipo proteins; poly(esters), poly(orthoesters), poly(urethanes) or poly(acrylamides), poly(glycolic acid), poly(lactic acid) and corresponding co-polymers (poly(lactide-co-glycolide acid; PLGA); siloxane, poly siloxane; dimethylsiloxane/methylvinylsiloxane copolymer; poly(dimethylsiloxane/methylvinylsiloxane/methylhydrogensiloxane) dimethylvinyl or trimethyl copolymer; silicone polymers; alkyl silicone; silica, aluminium silicate, calcium silicate, aluminium magnesium silicate, magnesium silicate, diatomaceous silica etc. as described more generally elsewhere herein), with cyclosporin to form mixture that may take the form of a suspension, solution or a colloid. The mixture is processed to form the composition, for example a minibead. For example the composition may be shaped into the desired form using a molding or hot-melt extrusion process to form beads.

Methods for preparing cores comprising cyclosporin and a water-soluble polymer matrix are described below. Generally these cores are coated with a modified release coating (and suitably sub-coating) to give the final modified release composition of the invention. The compositions may be prepared using known methods, for example methods analogous to those described in WO2010/133609, WO2011/018504, WO2012/069658 and PCT application number PCT/EP2013/064327, the disclosures of which are incorporated herein by reference.

Generally, the manufacturing processes described herein comprise mixing of liquids. Such mixing processes must be performed at temperatures at which the substances to be mixed in the liquid state are in liquid form. For example, thermoreversible gelling agents must be mixed at a temperature where they are in the liquid state, for example at a temperature of 50 to 75° C., for example 50 to 70° C., or 55-75° C., e.g. 60-70° C. and in particular embodiments about 55° C. or 65° C. in the case of mixing compositions comprising aqueous gelatin. Similarly other components of the composition may need to be heated to melt the component for example waxes or surfactants which may be used in the disperse phase.

Cores comprising a hydrogel-forming polymer and cyclosporin as disclosed herein may be made by mixing materials comprising for example water, a hydrogel-forming polymer and a surfactant to form an aqueous continuous phase, and mixing a disperse phase. At least one of the aqueous phase and the disperse phase comprises cyclosporin. Suitably both phases may be a clear liquid before they are mixed together. For example, the disperse phase may comprise cyclosporin (for example a disperse phase comprising an oil, an optional surfactant, cyclosporin and a surfactant) with the aqueous phase to form a colloid. The colloid may have the form of an emulsion or microemulsion wherein the cyclosporin disperse phase is dispersed in the aqueous continuous phase. The hydrogel-forming polymer is then caused or allowed to gel. Suitably, the process includes formulating or processing the core composition into a desired form, e.g. a minibead, which forming process may comprise moulding but preferably comprises ejecting the aqueous colloid through a single orifice nozzle to form droplets which are caused or allowed to pass into a cooling medium, e.g. a water-immiscible cooling liquid, in which the droplets cool to form for e.g. minibeads.

The mixing of the materials may comprise mixing an aqueous pre-mix (or aqueous phase) and a disperse phase pre-mix (e.g. oil phase pre-mix), wherein the aqueous pre-mix comprises water and water-soluble substances whilst the disperse phase pre-mix may comprise for example a vehicle containing an active ingredient. The vehicle may be a hydrophobic liquid, for example a liquid lipid, or it may be or comprise a material, for example a surfactant, for forming self-assembly structures. In particular, a disperse phase pre-mix may comprise cyclosporin, oil and other oil soluble components for example surfactant and optional solvents. The pre-mixes may contain one or more surfactants suitable for the phase they are to form, as previously mentioned.

The aqueous pre-mix comprises, or usually consists of, a solution in water of water-soluble constituents, namely the hydrogel-forming polymer and water-soluble excipient(s). The aqueous pre-mix may include a plasticiser for the hydrogel-forming polymer, as described elsewhere in this specification. The aqueous pre-mix may include a surfactant, e.g. to increase polymer viscosity and improve emulsification and thereby help prevent precipitation of active agent during processing. SDS is an example of such a surfactant. In any event, the constituents of the aqueous pre-mix may be agitated for a period sufficient to dissolve/melt the components, for example, from 1 hour to 12 hours to form the completed aqueous pre-mix.

The disperse phase pre-mix may comprise cyclosporin as a dispersion or preferably a solution in a vehicle as described above, for example in a liquid comprising an oil and/or surfactant as described above. For example the oil phase pre-mix may therefore be a liquid lipid, for example a medium chain triglyceride (MCT) composition, the medium chain triglyceride(s) being one or more triglycerides of at least one fatty acid selected from C₆-C₁₂ fatty acids and cyclosporin. Suitably the oil phase pre-mix is stirred at ambient temperature to form a solution of the cyclosporin in the oil. In some embodiments, the components of the oil phase pre-mix are mixed (or otherwise agitated) for a period of, for example, 10 minutes to 3 hours to form the pre-mix.

The two pre-mixes may be combined and agitated, for example for a period of a few seconds to an hour, for example from 30 seconds to 1 hour, suitably 5 mins to an hour, to form a dispersion of the disperse phase in an aqueous hydrogel-forming polymer, which dispersion may then be further processed to form the final formulation. The two pre-mixes may be combined into the dispersion by agitation in a mixing vessel; they may additionally or alternatively be combined in a continuous flow mixer.

The basic method for making a core comprising cyclosporin and hydrogel-forming polymer matrix, therefore, is to mix a liquid form (preferably a solution) of the hydrogel-forming polymer (or mixture of polymers) with the cyclosporin (and other disperse phase components) to form a dispersion in the polymer, which later in the process forms a hydrogel. The method normally comprises mixing together an aqueous polymer phase pre-mix and a disperse phase pre-mix. Taking account of the final composition required (as described elsewhere herein), the disperse phase pre-mix and the fluidic hydrogel-forming polymer (i.e. the solution or suspension of hydrogel-forming polymer) may be mixed in a weight ratio of from 1:1 to 1:10, particularly 1:4 to 1:9, e.g. 1:5 to 1:8, preferably approximately 1:7. In general, only gentle stirring of the components is required using a magnetic or mechanical system e.g. overhead stirrer as would be familiar to a person skilled in the art to achieve a dispersion of the disperse phase in the aqueous phase to form a colloid (which may be in the form of for example an emulsion or micro emulsion in which the aqueous hydrogel is the continuous phase). Continuous stirring is preferred. Mixing may also be achieved using an in-line mixing system. Any appropriate laboratory stirring apparatus or industrial scale mixer may be utilized for this purpose for example the Magnetic Stirrer (manufactured by Stuart) or Overhead Stirrer (by KNF or Fisher). It is preferred to set up the equipment in such a way as to minimise evaporation of contents such as, for example, water. In one embodiment of the process of the invention, it is preferred to utilise a closed system for stirring in order to achieve this aim. In-line mixing may be particularly suitable for closed system processing. Suitably mixing of the two components takes place at a temperature of 50 to 70° C., or 55-75° C., e.g. 60-70° C.

The mixing of the two phases results in a colloid wherein the aqueous hydrogel-forming polymer is an aqueous continuous phase and the component(s) not soluble in the aqueous phase, including cyclosporin are a disperse phase. The colloid may have the form of an emulsion or microemulsion.

In embodiments where the disperse phase is or comprises a surfactant, the amount of the surfactant in the disperse phase pre-mix may be selected such that upon combination of the disperse phase pre-mix with the aqueous pre-mix the surfactant concentration in the combined mixture exceeds the critical micelle concentration (CMC) for the surfactant used such that micelles are formed in the aqueous phase comprising the hydrogel-forming polymer. Depending on the concentration of surfactant used self-assembly structures other than micelles may also form. The CMC for a particular surfactant may be determined using well known methods, for example as described in Surfactants and Polymers in Aqueous Solutions Second Edition, Chapter 2, Holmberg et al. In embodiments mixing of the aqueous and disperse phase which is or comprises a surfactant may result in the formation of a clear liquid, for example a microemulsion, in which the aqueous phase comprising the hydrogel-forming polymer is the continuous phase. Microemulsions are a thermodynamically stable dispersion of self-assembly structures in the aqueous phase, the size of the self-assembly structures being sufficiently small to give a transparent appearance. The size of the self-assembly structures present as the disperse phase resulting from the mixing of the aqueous and surfactant phases may be from about 0.5 nm to 200 nm, for example about 1 nm to 50 nm, or about 5 nm to 25 nm. The size of the self-assembly structures formed and other characteristics such as the optical isotropicity of the composition (for example a microemulsion) may be determined using well known techniques such as dynamic light scattering.

Where the polymer matrix substantially consists of gelatin with the addition of sorbitol, the aqueous phase of polymer matrix is prepared by adding the appropriate quantities of sorbitol (and surfactant if desired) to water, heating to approximately 50 to 75° C., for example 60-75° C. until in solution and then adding gelatin although the precise order and timing of addition is not critical. A typical “gelatin solution” comprises 8 to 35%, (for example 15-25%, preferably 17-18%) gelatin; 65%-85% (preferably 77-82%) of water plus, optionally, from 1-5% (preferably 1.5 to 3%) sorbitol. When present surfactant (e.g. anionic surfactant) in the aqueous phase pre-mix may be present in an amount of 0.1 to 5% (preferably 0.5 to 4%) wherein all parts are by weight of the aqueous phase.

Optionally the processing temperature required for standard gelatin can be reduced to a desirable target temperature e.g. 37° C. by use of lower melting-point gelatin (or gelatin derivatives or mixtures of gelatins with melting point reducers) or other polymer matrix material such as, for example, sodium alginate. If gelatin droplets are being formed by machine extrusion and immediately cooled e.g. in a cooling bath, additional appropriate inlet tubing can be used to introduce an oil phase containing cyclosporin at ambient temperature into the hotter fluid gelatin solution (and the mixture can be immediately homogenized) very shortly before ejection from a beading nozzle or other dropletting process such that the duration of exposure of the cyclosporin to the higher temperature gelatin is limited so reducing the degree of any heat-dependent degradation of the active ingredient. This process may use any appropriate device such as, for example, a homogenizer, e.g. a screw homogenizer, in conjunction with an extrusion-type apparatus as described for example in WO 2008/132707 (Sigmoid Pharma) the entirety of which is incorporated herein by reference.

The colloid is formed by combining the disperse phase pre-mix with the liquid aqueous phase with stirring as described above. The resultant colloidal dispersion then has the composition of a solidified core described above but with liquid water still present in the core composition.

Optionally the cyclosporin may be added after mixing the aqueous phase and other components of the disperse phase of the type comprising a vehicle in addition to the cyclosporin, however, it is preferred that the cyclosporin is added together with any other components of the disperse phase as a pre-mix.

The resulting colloid is then poured or introduced into a mould or other vessel or poured onto sheets or between sheets or delivered dropwise (or extruded) into another fluid such that the polymer matrix-containing aqueous phase, on solidification, takes the form of the mould, vessel, sheet or droplet/bead intended. It is preferred to progress to mould-forming e.g. beading without delay.

Solidification (gelling) can occur in a variety of ways depending on the polymer of the matrix, for example by changing the temperature around the mould, vessel, sheet, droplet/bead etc. or by applying a solidification fluid or hardening solution so that the moulded shape is gelled or solidified. In certain embodiments both temperature change and application of a solidifying fluid or hardening solution are employed together or simultaneously.

In the preferred embodiment in which the core comprising cyclosporin takes the form of minibeads, the minibeads may be formed for example by dropping the colloid dropwise into a fluid which effects solidification. Where the viscosity of the composition to be beaded reaches a certain point, drop formation becomes more difficult and specialised apparatus is then preferred.

By use of the term “dry”, it is not sought to imply that a drying step is necessary to produce the dry core (although this is not excluded) rather that the solid or solidified aqueous external phase is substantially free of water or free of available water. Solidification of the aqueous phase (external phase) may have arisen through various means including chemically (e.g. by cross-linking) or physically (e.g. by cooling or heating). In this respect, the term “aqueous phase” is nevertheless employed in this document to denote the external (continuous) phase of the core even though water, in certain embodiments, is largely absent from (or trapped within the cross-linked matrix of) the core. The external phase of the core is however water-soluble and dissolves in aqueous media.

In the case where solidification can be achieved by raising or reducing temperature, the temperature of the solidification fluid can be adapted to achieve solidification of the core at a desired rate. For example, when gelatin is used as the hydrogel-forming polymer, the solidification fluid is at a lower temperature than the temperature of the emulsion thus causing solidification i.e. gelling of the polymer matrix. In this case, the solidification fluid is termed a cooling fluid.

In the case where solidification can be achieved chemically, e.g. by induction of cross-linking on exposure to a component of the solidification fluid, the concentration of such component in the solidification fluid and/or its temperature (or other characteristic or content) can be adjusted to achieve the desired rate and degree of solidification. For example, if alginate is chosen as the polymer matrix, one component of the solidification fluid may be a calcium-containing entity (such as, for example, calcium chloride) able to induce cross-linking of the alginate and consequent solidification. Alternatively, the same or similar calcium-containing entity may be included (e.g. dispersed) in the aqueous phase of the fluid emulsion prior to beading and triggered to induce cross-linking e.g. by applying a higher or lower pH to a solidification fluid into which droplets of emulsion fall dropwise or are introduced. Such electrostatic cross-linking can be varied as to the resulting characteristics of the minibead by control of calcium ion availability (concentration) and other physical conditions (notably temperature). The solidification fluid may be a gas (for example air) or a liquid or both. For example, when gelatin is used as the hydrogel-forming polymer matrix, the solidification fluid can be initially gaseous (e.g. droplets passing through cooling air) and then subsequently liquid (e.g. droplets passing into a cooling liquid). The reverse sequence may also be applied while gaseous or liquid cooling fluids alone may also be used. Alternatively, the fluid may be spray-cooled in which the emulsion is sprayed into a cooling gas to effect solidification.

In the case of gelatin or other water-soluble polymer (or polymer mixture) destined to form an immobilization matrix, it is preferred that the solidification fluid be a non-aqueous liquid (such as, for example, medium chain triglycerides, mineral oil or similar preferably with low HLB to ensure minimal wetting) which can conveniently be placed in a bath (cooling bath) to receive the droplets of the colloid as they solidify to form the minibeads of the core. Use of a non-aqueous liquid allows greater flexibility in choice of the temperature at which cooling is conducted.

Where a liquid cooling bath is employed, it is generally maintained at less than 20° C., preferably maintained in the range 5-15° C., more preferably 8-12° C. when standard gelatin is used as the hydrogel-forming polymer. If a triglyceride is chosen as the cooling fluid in the cooling bath, a preferred example is Miglyol 810 from Sasol.

If alginate is selected as the polymer matrix, a typical method of making minibeads involves dropwise addition of a 3% sodium alginate solution in which oil droplets are dispersed as described above into a 4° C. crosslinking bath containing 0.1 M calcium chloride to produce calcium alginate (this method can be referred to as “diffusion setting” because the calcium is believed to diffuse into the minibeads to effect cross-linking or setting). Using a syringe pump, or Inotech machine, droplets can be generated or extruded (e.g., at 5 mL/h if a pump is used) through a sterile needle or other nozzle (described elsewhere herein) which can be vibrating as discussed elsewhere herein. Airflow of between 15 and 20 L/min through 4.5 mm tubing can be applied downwards over the needle to reduce droplet size if desired. Newly formed minibeads can then be stirred in the calcium chloride bath for up to an hour. If carrageenan is used as the polymer matrix both salt and reduction in temperature e.g. by dropping into cooling oil may be used to obtain solidification.

An alternative approach when using alginate is internal gelation in which the calcium ions are dispersed in the aqueous phase prior to their activation in order to cause gelation of hydrocolloid particles. For example, this can be achieved by the addition of an inactive form of the ion that will cause crosslinking of the alginate, which is then activated by a change in e.g. pH after sufficient dispersion of the ion is complete (see Glicksman, 1983a; Hoefler, 2004 which are both incorporated herein by reference). This approach is particularly useful where rapid gelation is desired and/or where the diffusion approach may lead to loss of drug by diffusion thereof into the crosslinking bath.

Where another ionotropic polymer is used than alginate, suitable analogous processes may be used to those described herein in relation to alginate.

Following shape-forming, moulding or beading, the resultant shapes or forms may be washed then dried if appropriate. In the case of minibeads solidified in a solidification fluid, an optional final step in the method of production described above therefore comprises removal of the solidified minibeads from the solidification fluid. This may be achieved e.g. by collection in a mesh basket through which the solidification fluid (e.g. medium chain triglycerides) is drained and the minibeads retained and is preferably conducted without delay e.g. as soon as the minibeads have formed or within 5, 10, 15, 20, 25 or 30 minutes of their formation. Excess solidification fluid may then be removed using a centrifuge (or other apparatus or machine adapted to remove excess fluid) followed by drying of the minibeads to remove water or free water and/or removal of some or all of any additional solvent e.g. ethanol or isopropyl alcohol used to dissolve or facilitate dissolution of the active principle in preceding steps optionally followed by washing (e.g. using ethyl acetate) and a subsequent “drying” step to remove excess solvent (e.g. ethyl acetate). Isopropyl alcohol is an example of a solvent which is preferably removed later in processing to reduce residues in the oil or aqueous phase. Drying can be achieved by any suitable process known in the art such as use of a drum drier (e.g. Freund Drum dryer which may be part of the Spherex equipment train if used) with warm air at between 15° C. and 25° C., preferably around 20° C. leading to evaporation or entrainment of the water by the air. Alternatively, drying may be carried out using of a fluid bed drier (e.g. Glatt GPCG 1.1) with warm air between 40° C. and 60° C. Use of gelatin as the polymer matrix (e.g. as principal constituent of the aqueous immobilisation phase) in most cases requires a drying step and for minibeads this is preferably achieved by drying in air as above described. The resultant composition (the composition of the invention) is essentially dry as described in more detail above.

In general, the minibeads may be generated by the application of surface tension between the liquid dispersion (the mixture of the aqueous and surfactant phases) and an appropriate solidification fluid such as, for example, gas or liquid in order to create the spherical or substantially spherical shape of the ultimate minibeads.

Alternatively, the minibeads may be produced through ejection or extrusion of the liquid dispersion through an orifice or nozzle with a certain diameter and optionally subject to selected vibration (using selected frequencies) and/or gravitational flow. Examples of apparatus which may be used to form the minibeads include encapsulation prilling, drop pelletising, spray cooling or spray congealing apparatus, for example, the Freund Spherex, ITAS/Lambo, Globex, Inotech, GEA Niro, Droppo, Buchi, Gelpell processing equipment. Operation of the Spherex apparatus manufactured by Freund as may be desired to manufacture minibeads according to the present invention is described in U.S. Pat. No. 5,882,680 (Freund), the entire contents of which are incorporated herein by reference. It is preferred to select a vibrational frequency in the region of 2-200 Hz suitably 10-15 Hz although the ultimate choice (and separately the amplitude of vibration selected) depends on the viscosity of the dispersion to be beaded. If the polymer matrix is chosen to solidify at lower temperature, it may be appropriate to maintain the lines to the orifice/nozzle at a certain temperature to maintain the fluidity of the solution. Suitably the colloid is ejected through a single-orifice nozzle, e.g. having a diameter of from 0.1 mm to 5 mm (for example 0.5-5 mm), to form drops which are then caused or allowed to fall into a cooling oil or other hardening medium and allowed to harden to form seeds, after which the seeds are recovered from the cooling oil and dried.

It will be appreciated, therefore, that the invention includes a process for manufacturing a core comprising cyclosporin in a polymer matrix which comprises: forming an aqueous pre-mix which comprises water and water-soluble/dispersible materials (including therefore a hydrogel-forming polymer) and a disperse pre-mix (e.g. an oil phase pre-mix) which comprises cyclosporin and optionally a vehicle and other excipients (e.g. oil(s) and oil soluble/dispersible materials), and combining the two pre-mixes to form a colloid (disperse phase) within an aqueous phase comprising the hydrogel-forming polymer. The colloid may then be formed into a shaped unit, for example a minibead to provide the core comprising the cyclosporin. More particularly the manufacture of a core comprising cyclosporin and a polymer matrix (suitably a hydrogel-forming polymer matrix may comprise:

(i) forming an aqueous phase pre-mix comprising a solution in water of water-soluble constituents (e.g. of a hydrogel forming polymer, any water-soluble excipient(s), as described elsewhere herein);
(ii) forming a disperse phase pre-mix typically comprising a dispersion or preferably a solution of cyclosporin in a liquid, optionally where the liquid is an oil (and optionally together with other disperse phase constituents (e.g. surfactant, solvents etc as described elsewhere herein));
(iii) mixing the aqueous phase pre-mix (i) and the disperse phase pre-mix (ii) to form a colloid;
(iv) ejecting the colloid through a nozzle to form droplets;
(v) causing or allowing the a hydrogel forming polymer to gel or solidify to form a water-soluble polymer matrix; and
(vi) drying the solid.

As a further aspect of the invention there is provided a composition obtainable by (having the characteristic of) any of the processes described herein. It is to be understood that the processes described herein may therefore be used to provide any of the specific cores described in embodiments herein by dispersing the appropriate components which form the disperse phase of the core in the appropriate components which form the aqueous continuous matrix phase of the core.

The preceding paragraphs describe the formation of uncoated cores comprising cyclosporin in for example a hydrogel-forming polymer matrix. The cores are suitably coated to provide the modified release composition according to the invention. Suitably the cores are first coated with a subcoat and is then further coated with a modified release coating. Optionally the composition is further coated with an outer protective coating as described herein. Suitable sub coats, modified release coatings and outer protective coatings are any of those described herein. The coating(s) may be applied using well known methods, for example spray coating as described below to give the desired sub coat and modified release coating weight gains.

With regard to one of the methods described above (ejection of emulsion through an optionally vibrating nozzle) with two concentric orifices (centre and outer), the outer fluid may form a coating (outside the minibead) as described herein. The Spherex machine manufactured by Freund (see U.S. Pat. No. 5,882,680 to Freund) is preferably used (the entire contents of this patent is incorporated herein by reference). Other similar ejection or extrusion apparatus may also be used, for example the ejection apparatus described hereinbefore.

Use of the Spherex machine achieves very high monodispersity. For example, in a typical 100 g, batch 97 g of minibeads were between 1.4 to 2 mm diameter or between 1 and 2 mm. Desired size ranges can be achieved by methods known in the art for rejecting/screening different sized particles. For example, it is possible to reject/screen out the larger/smaller minibeads by passing a batch first through e.g. a 2 mm mesh and subsequently through a 1.4 mm mesh.

The 1.4 to 2 mm diameter range is a good size if it is desired to spray coat the minibeads (if smaller, the spray of the coating machine may bypass the minibead; if too large, the minibeads may be harder to fluidise which is necessary to achieve consistent coating).

### Coating Process

The coating process can be carried out by any suitable means such as, for example, by use of a coating machine which applies a solution of a polymer coat (as described above in particular) to the composition. Polymers for coating are either provided by the manufacturer in ready-made solutions for direct use or can be made up before use following manufacturers' instructions.

Coating is suitably carried out using a fluid bed coating system such as a Wurster column to apply the coating(s) to the cores. Appropriate coating machines are known to persons skilled in the art and include, for example, a perforated pan or fluidized-based system (including top spray, bottom spray and radial spray variants). Specific examples include the GLATT, Vector (e.g. CF 360 EX), ACCELACOTA, Diosna, O'Hara, Huttlin and/or HICOATER processing equipment. To be mentioned is the MFL/01 Fluid Bed Coater (Freund) used in the “Bottom Spray” configuration.

Typical coating conditions are as follows:

| Process Parameter | Values |
|---|---|
| Fluidising airflow (m3/h) | 20-60 (preferably 30-60) |
| Inlet air temperature (° C.) | 20-65 |
| Exhaust air temperature (° C.) | 20-42 |
| Product temperature (° C.) | 20-45 (preferably 40 to 42) |
| Atomizing air pressure (bar) | Up to 1.4 e.g. 0.8-1.2 |
| Spray rate (g/min) | 2-10 and 3-25 RPM |

Suitably the coating is applied as a solution or dispersion of the polymers (and other components) of the coating. Generally the coatings are applied as an aqueous, solution of dispersion, although other solvent systems may be used if required. The coating dispersion is applied to the cored as a spray in the fluid bed coater to give the required coating weight gain. Generally the coating process is carried out at a temperature which maintains the cores at a temperature of from 35 to 45° C., preferably 40 to 42° C.

After applying the coating, the composition may be dried, for example by drying at 40 to 45° C.

The invention further provides a product having the characteristics of a composition obtained as described herein, a product defined in terms of its characteristics being defined by the characteristics of the composition to the exclusion of the method by which it was made.

As mentioned herein the processes described may be used to provide any of the compositions described in the various embodiments herein. By way of example there is provided a modified release composition of the invention comprising a core and a modified release coating wherein the core comprises a hydrogel forming polymer matrix comprising gelatin, cyclosporin, medium chain mono-di- or tri-glycerides, a co-solvent and surfactant, the core having the characteristics of a core obtained by the process comprising steps (i) to (vi) described above for forming the core, wherein the aqueous phase pre-mix in step (i) of the process comprises gelatin and surfactant (suitably an anionic surfactant), and the disperse phase pre-mix in step (ii) of the process comprises medium chain mono-di- and/or tri-glycerides, cyclosporin, surfactant (suitably a non-ionic surfactant) and solvent (for example 2-(2-ethoxyethoxy)ethanol e.g. Transcutol P); and the wherein the core is optionally coated with a first coating (sub coating) comprising a water-soluble cellulose ether or a water-soluble derivative of a cellulose ether and the optionally sub-coated core is coated with a modified release coating; wherein the first coating (subcoating) and the modified release coating are any of those described herein.

In the cores described herein to which the following characteristics are applicable, e.g. in the immediately preceding paragraph, the following characteristics may be present:

gelatin may be present in an amount of in an amount of 300 to 700 mg/g;

the medium chain mono-, di- or tri-glycerides (for example caprylic/capric triglyceride) may be present in an amount of 20 to 200 mg/g;

co-solvent (for example 2-(ethoxyethoxy)ethanol) may be present in an amount of 150 to 250 mg/g;

non-ionic surfactant (for example sorbitan-based surfactants, PEG-fatty acids, or glyceryl fatty acids or poloxamers or particularly a polyethoxylated castor oil for example Kolliphor™ EL) may be present in an amount of 80 to 200 mg/g;

anionic surfactant (for example, alkyl sulfates, carboxylates or phospholipids (particularly SDS)) may be present in an amount of 15 to 50 mg/g; and

cyclosporin may be present in an amount of from 60 to 150 mg/g, suitably 80 to 100 mg/g, for example 81 to 98 mg/g;

wherein all weights are based upon the dry weight of the core before coating.

Preferably the core above, or any of the cores described herein are coated with a first coating (sub-coating) and a modulated release coating outside the first coating; wherein the first coating is or comprises a water-soluble cellulose ether or a water-soluble derivative thereof, particularly hydroxypropylmethyl cellulose; the first coating being present in an amount corresponding to a weight gain due to the first coating in a range selected from: (i) from 1% to 20%; (ii) from 8% to 12%, for example about 10%; (iii) from 4% to 6%, for example about 5%; or (iv) about 6% to about 10%, for example about 7%, about 7.5%, about 8%, about 8.5%, about 9% or about 9.5 by weight based upon the weight of the core prior to applying the first coating; and wherein

preferably, any modified release coating, especially in the embodiments of the immediately preceding paragraphs, is or comprises a pH independent modified release coating, more especially a modified release coating comprising ethyl cellulose (e.g. Surelease™) still more particularly a modified release coating comprising ethyl cellulose and optionally a water-soluble polysaccharide, for example pectin (e.g. a Surelease™ pectin coating as described herein); and wherein the second coating (modified release coating) is present in an amount corresponding to a weight gain of the composition due to the second coating selected from (a) from 10% to 12%, for example about 11% or about 11.5%; or (b) from about 8% to about 12%, for example about 8.5%, about 9%, about 9.5%, about 10%, about 10.5% or about 11% by weight based upon the weight of the composition prior to applying the second coating.

### Other Therapies

In addition to treating the patient with the modified release composition comprising cyclosporin and any active agent(s) as described herein, the patient may be treated with another drug (e.g. 2 drugs) suitable for use in the treatment of ulcerative colitis. Additional drugs may be for example, an immunosuppressant or a biologic (for example an antibody) therapy suitable for use in treating ulcerative colitis.

The immunosuppressant may be a thiopurine immunosuppressant, for example azathioprine or 6-mercaptopurine.

The immunosuppressant may be a calcinuerin inhibitor suitable for use in the treatment of ulcerative colitis, for example cyclosporin (in addition to the cyclosporin comprised in the modified release composition), tacrolimus or sirolimus.

The additional drug (or drugs) may be a biological therapy (“a biologic”, for example an antibody, an antibody fragment (for example an antigen binding fragment), or an antibody fusion protein and the like), or a small molecule drug, in either case suitable for the treatment of ulcerative colitis. Examples of antibody therapies suitable for treating ulcerative colitis include anti-TNF antibody therapies, for example infliximab, adalimumab or golimumab and integrin inhibitor antibody therapies such as vedolizumab.

In some embodiments of the patient may be treated with the composition as described herein and additionally treated with one or more of the said additional therapies. For example a patient may be treated concurrently with the composition, the active agent and biological therapy, wherein the biological therapy is suitable for use in the treatment of ulcerative colitis (for example infliximab, adalimumab, golimumab or vedolizumab).

For example, in some embodiments the patient may be treated with the modified release composition comprising cyclosporin as described herein and be treated concurrently with an additional source of cyclosporin, wherein the additional source of cyclosporin is not a modified release composition comprising cyclosporin. The additional source of cyclosporin may for example be intravenously administered cyclosporin, cyclosporin administered as an enema, or an orally administered cyclosporin formulation optionally (for example an instant release cyclosporin formulation such Neoral™ or Sandimmun™).

In other embodiments the patient may treated concurrently with the composition as described herein and is not treated with an immunosuppressant or a biologic.

In other embodiments the patient may treated concurrently with the composition as described herein and is not treated with a thiopurine immunosuppressant for example azathioprine or 6-mercaptopurine.

In other embodiments the patient may treated concurrently with the composition as described herein and is not treated with a biological therapy is suitable for use in the treatment of ulcerative colitis (for example infliximab, adalimumab or golimumab).

### Patient

The modified release composition of the invention may be used in the treatment of a mammal for example a human. In one aspect the patient is a human. In one aspect the patient is an adult human (aged 18 years or more). In another aspect the patient is a human aged less than 18 years.

### EXAMPLES

### Example 1: Preparation of a Minibead Having a Surelease/Pectin Coating

The minibead was generally prepared by forming a core according to the following procedure and then coating the core with a mixture of Surelease® (an ethylcellulose dispersion) and Pectin in a ratio of 98:2 (Surelease:Pectin) in a weight gain of 9% relative to the weight of the core.

### Core Manufacture

The cores in the form of seamless minibeads were prepared using Spherex process as follows.

An aqueous phase was prepared by mixing sodium dodecyl sulphate (SDS) and D-sorbitol with purified water under constant stirring. Gelatin was then added to this solution and gentle heat was applied to approximately 60-70° C. to achieve complete melting of gelatin.

An oil phase was prepared by mixing together 2-(2-ethoxyethoxy)ethanol (Transcutol HP), polyethoxylated castor oil (Kolliphor EL) and capric/caprylic triglyceride (Miglyol 810) with stirring at room temperature to form a solution. Ciclosporin A was added and mixed until a clear solution was obtained. The oil phase was mixed with the heated aqueous phase in a ratio of approximately 1:7 (oil phase:aqueous phase). The resulting mixture was stirred at 60-70° C. to achieve homogeneity.

The resulting mixture was then fed (via temperature controlled tubing) through a vibrating nozzle, with a single nozzle outlet with a diameter of 3 mm. Seamless minibeads were formed as the solution flowed through the vibrating nozzle into a cooling chamber of constantly flowing medium chain triglyceride (Miglyol 810) cooling oil at a temperature of 10° C.

The minibeads were removed from the cooling oil and placed in a centrifuge to remove the excess oil. Following centrifugation, a first drying step was initiated with a set refrigerator temperature of 10° C. and the heater temperature of 20° C. The dryer was rotated at 15 RPM. When the beads were observed to be freely rotating in the drying drum, they were considered to be dry.

The minibeads were washed with ethyl acetate and then dried for a further 24 h under the same drying conditions as those mentioned above in the first drying step. The dried minibeads were then sieved to remove oversize and undersize beads resulting in cores 1 mm-2 mm in diameter. This procedure provided cores with the composition shown in Table 1, the values being the weight percent of the total weight for each component.

TABLE 1

| Component | w/w % |
|---|---|
| Cyclosporin A | 10.8 |
| Miglyol 810 N | 4.6 |
| Transcutol HP | 16.4 |
| Kolliphor EL | 9.2 |
| SDS | 4.0 |
| Sorbitol | 5.7 |
| Gelatin | 49.3 |

### Coating the Core

The Surelease®/pectin coating was applied by the following procedure. Pectin was added to purified water in a stainless steel vessel and mixed to obtain a solution. Surelease® was slowly added to the vessel whilst maintaining mixing to provide the required Pectin ratio of Surelease® to Pectin (98:2) for the coating. The resulting coating suspension was then applied onto the surface of minibead cores loaded into a fluid bed coater (Wurster column). The processing parameters, such as inlet air temperature and inlet air volume, were adjusted to keep the minibead temperature between 40° C. and 42° C. until the required coating weight gain of 9% was reached. The resulting coated minibeads were dried in the coater for an hour at 40-45° C. Minibeads with the composition shown in Table 2 below were produced by the above procedure.

TABLE 2

| Component | w/w % |
|---|---|
| Cyclosporin A | 9.9 |
| Miglyol 810 N | 4.2 |
| Transcutol HP | 15.1 |
| Kolliphor EL | 8.4 |
| SDS | 3.7 |
| Sorbitol | 5.2 |
| Gelatin | 45.2 |
| Surelease | 8.1 |
| Pectin | 0.2 |

### Example 2: Evaluation of the Efficacy of the Minibeads of Example 1

CyCol® (a composition comprising minibeads of Example 1) was administered to human subjects in a randomized, double-blind, placebo-controlled study of the controlled release minibead formulation of ciclosporin shown in Table 2 in the treatment of mild to moderate ulcerative colitis.

The study was a multicentre, randomized, double-blind, placebo controlled, 2-parallel group study. Subjects were recruited from Inflammatory Bowel Disease (IBD) clinics in the Republic of Ireland and the United Kingdom. 118 subjects were randomized with 1 subject deemed non-eligible. The intention to treat population was therefore 117 subjects.

After screening, eligible subjects were randomized at baseline (Day 0) with 53 subjects receiving an oral dose of 75 mg (3×25 mg capsules) of CyCol®, a controlled release minicapsule formulation of ciclosporin, once daily and 65 subjects receiving placebo (3 visually matching capsules) once daily for 4 weeks.

Subjects were evaluated at clinic visits at baseline (Day 0), Week 4 (end of treatment period), and follow-up. At Weeks 1 and 2, subjects were contacted by telephone to assess adverse events (AEs) and concomitant medication usage. The follow up visit took place approximately 4 weeks after the last dose of study drug.

At the clinic visit at Week 4, subjects underwent UC clinical assessment (i.e., documentation of stool frequency, rectal bleeding and investigator rating of disease activity), complete physical examination, concomitant medication monitoring, vital signs (including blood pressure, heart rate, temperature, and respiratory rate), AE assessment, and laboratory assessments including a flexible sigmoidoscopy with biopsies to assess mucosal appearance and histology. Disease Activity was determined within 1 week of Day 0 and at Week 4.

The following criteria were required to be met for a subject to be eligible for the study:

1. Male and female subjects aged >18 years.

2. Subjects with a mild to moderate diagnosis of UC involving at least the rectum and sigmoid colon (i.e., ulcerative proctosigmoiditis, left-sided ulcerative colitis or pancolitis), determined by historical (>3 months prior to Day 0) colonoscopy or, sigmoidoscopy, and confirmed with biopsies.

3. Clinical severity assessed at screening using the Disease Activity Index (DAI, Table 1) of 4 to 10, inclusive with a muscosal sub-score ≥1 (within 8 days of Day 0).

4. Clinical severity must be documented and confirmed by flexible Sigmoidoscopy, with a mucosal subscore of ≥1, within 8 days of starting study treatment (i.e., Day 0)

5. Subjects must sign and date a written informed consent.

6. Subjects must agree not to change the dosing regimen of any current UC medications (e.g., low dose steroids (e.g. 10 mg daily prednisolone, 5-ASA compounds, or immunomodulatory agents, namely purine analogues) from screening until the end of the 4-week treatment period.

7. Subjects must agree to refrain from intake of St. Johns Wort or any other prescription, over-the-counter, or herbal preparation that is known to affect cytochrome P450 metabolism throughout the 4-week treatment period of the study.

8. Subjects must agree to refrain from intake of grapefruit or grapefruit juice or any other food or drink that is known to affect cytochrome P450 metabolism throughout the 4 week treatment period of the study.

If the following criteria were met a subject was not eligible for the study:

1. Subjects with severe or fulminant UC.

2. Subjects with UC limited to rectum only.

3. Subjects who have had any previous colonic surgery.

4. Subjects who have any histological evidence of dysplasia on colonoscopic biopsy.

5. Women of childbearing potential who are unable or unwilling to use adequate contraceptive methods to avoid pregnancy.

6. Subjects who have failed on previous ciclosporin therapy.

7. Subjects who have had any biologic therapy within the past 2 months prior to Day 0.

8. Subjects who have had methotrexate therapy within the past 4 weeks prior to Day 0.

9. Subjects who have had a steroid treatment dose of greater than 10 mg/day prednisolone (or equivalent) within the past 4 weeks prior to Day 0.

10. Subjects who have had topical treatment (e.g. enemas) within 4 weeks of Day 0 and must refrain from taking topical treatments for UC from the screening visit until the end of the 4-week treatment period.

11. Subjects with significant renal impairment, hepatic impairment, uncontrolled hypertension, premalignant skin lesions or current malignancies, or any other severe co-morbid condition.

12. Subjects with any known hypersensitivity to ciclosporin or any of its excipients.

13. Subjects with a positive screening stool assay for Clostridium difficile, hemorrhagic E. coli 0157:H7, Salmonella or Shigella

14. Subjects with a diagnosis of Crohn's colitis, ischemic colitis, NSAID-induced colitis, or radiation colitis.

The primary objective of this study was to evaluate the efficacy of the minibeads of Example 1 in inducing clinical remission in subjects with mild to moderate UC involving at least the rectum and sigmoid colon. Remission was defined as a Disease Activity Index (DAI) score ≤2 after 4 weeks of treatment with no-individual DAI sub-score >1 at Week 4. Subjects in remission by this definition had a rectal bleeding DAI sub-score of either 0 or 1.

The secondary objective of the study was to evaluate the efficacy of the minibeads of Example 1 based on mucosal healing with an absolute mucosal appearance subscore of 0 or 1, the reduction in DAI score of ≥3 at Week 4 and with a decrease in the rectal bleeding sub-score of ≥1 or an absolute rectal bleeding sub-score of 0 or 1, and histological healing. Such a reduction in DAI is categorised as a clinical response.

The definitions of remission and response used are consistent with the definitions established in Dignass A, et al, Second EUROPEAN evidence-based Consensus on the diagnosis and management of ulcerative colitis: Definitions and diagnosis, Journal of Crohn's and Colitis (2012).

Disease activity index was assessed using the scoring system shown in Table 3 below.

TABLE 3

| | | | | |
|---|---|---|---|---|
| Normal | Mild | Moderate | Severe | Total |
| (Score = 0) | (Score = 1) | (Score = 2) | (Score = 3) | Score |

| | | | | |
|---|---|---|---|---|
| Rectal | None | Streaks of blood | Obvious blood | Mostly blood |
| Bleeding | | | | |
| Stool | Normal | 1-2/day > normal | 3-4/day > normal | >4/day > normal |
| Frequency | | | | |
| Mucosal | Normal | Erythema | Marked erythema | Ulceration |
| appearance | | Decreased | Friability | Spontaneous |
| | | vascular pattern | Granualarity | bleeding |
| | | Minimal | Absent vascular | |
| | | granularity | pattern | |
| | | | Bleeding or | |
| | | | minimal trauma | |
| | | | No ulceration | |
| Physician's | Normal | Mild | Moderate | Severe |
| Global | | | | |
| Assessment | | | | |

Subjects in the study received either 25 mg cyclosporin capsules containing minibeads of Example 1 or visually matching placebo capsules. The subjects were instructed to take 3 capsules whole, once daily by mouth 30 minutes before taking food, for 4 weeks. All 3 capsules were to be taken together. It is noteworthy that the dose of ciclosporin is relatively low, 75 mg, approximately equivalent to 1 mg/kg dose in a person of average weight (70 kg) compared to 10-15 mg/kg for organ transplantation or 2.5-5 mg/kg for other indications.

The study contained 117 subjects in the intention-to-treat (ITT) population; 64 subjects received placebo and 53 received cyclosporin treatment (labelled as CyCol® in the tables and figures) with capsules containing the minibeads of Example 1. Table 4, shown below, indicates the medications for ulcerative colitis taken by the subjects during the study treatment period concurrently with the study treatment or placebo.

TABLE 4

| | CyCol ® | | Placebo | |
|---|---|---|---|---|
| | (N = 53) | | (N = 64) | |
| | N | (%) | N | (%) |
| Treatment | On Day 0 through Day 28 | | | |
| 5-ASA | 34 | (64.1) | 37 | (57.8) |
| Immunomodulators | 0 | (0) | 1 | (1.6) |
| Steroids | 0 | (0) | 1 | (1.6) |
| 5-ASA + Steroids | 5 | (9.4) | 5 | (7.8) |
| 5-ASA + Immunomodulators | 8 | (15.1) | 11 | (17.2) |
| 5-ASA +Steroid + Immunomodulators | 1 | (1.9) | 2 | (3.1) |
| No UC treatment | 10 | (18.9) | 12 | (18.8) |

N = number of subjects,
% = calculation based on N

### Clinical Remission in Subjects (Primary Study Objective)

Table 5, shown below, presents the statistical comparison of the remission rates of the ITT population between the CyCol® treatment group and the placebo treatment group based on the DAI. Subjects were classified as being in remission when the subject's DAI scores ≤2 after 4 weeks of treatment with no-individual DAI sub-score >1 at Week 4. Subjects in remission by this definition had a rectal bleeding DAI sub-score of either 0 or 1.

TABLE 5

| | | | Fisher's exact |
|---|---|---|---|
| | CyCol ® | Placebo | test |
| n/N | 7/53 | 4/64 | p = 0.2211 |
| % | 13.2 | 6.3 | |

n = number of subjects in remission,
N = number of subjects,

The data shown in Table 5 is represented as a bar chart in FIG. 1.

The remission rate was numerically higher in the CyCol® group (13.2%) compared to placebo (6.3%). However, the difference was not statistically significant (p=0.2211). A statistically significant result is defined as having a Fischer's exact test p value of 0.05 or less.

### Clinical Response in Subjects (Secondary Study Objective)

Table 6, shown below, presents the number of subjects achieving clinical response; compared between the treatment groups. Such subjects had a reduction in DAI score of ≥3 at Week 4 with a decrease in the rectal bleeding subscore of ≥1 or an absolute rectal bleeding subscore of 0 or 1.

TABLE 6

| | | | Fisher's exact |
|---|---|---|---|
| | CyCol ® | Placebo | test |
| n/N | 15/53 | 9/64 | p = 0.0684 |
| % | 28.3 | 14.1 | |

n = number of subjects in response,
N = number of subjects,

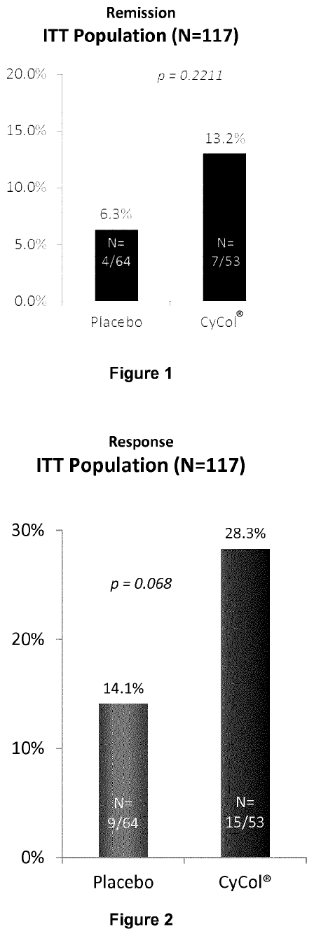

The data shown in Table 6 is represented as a bar chart in FIG. 2.

The rates for the DAI score reduction were numerically higher in the CyCol® group; however, the results were not statistically significant (p=0.0684). A statistically significant result is defined as having a Fischer's exact test p value of 0.05 or less. The difference between the rate of remission in the placebo and CyCol® treatment group (Δ) is 14.2.

### Example 3: Remission and Response in Subjects with Moderate Ulcerative Colitis

Data collected during the study was analysed following the data analysis convention described in Table 7 below.

TABLE 7

Alternative Analysis Convention
ITT group includes all patients and those with a
missing clinical efficacy data considered as non-responders
Subgroups were defined solely on the UC
medications the patients were receiving when study
medication was started (i.e. baseline) and through
Day 28 (end of study treatment).
Medications intravenous, rectal and oral routes
were considered as relevant UC medications.
The start dates if missing were imputed as 1^{st} of the
month. The missing stop dates were imputed as
27^{th} of the month.

In the ITT population, following the Alternative Analysis Convention, there were 93 subjects classified as being in a population subgroup having moderate ulcerative colitis (defined as a DAI of 6 or more). The remission rate and response rate for this population subgroup for CyCol® and placebo therapy are shown in Table 8.

TABLE 8

| | | | | Fisher's exact |
|---|---|---|---|---|
| | | CyCol ® | Placebo | test |
| Remission | n/N | 3/40 | 1/53 | p = 0.3109 |
| | % | 7.5 | 1.9 | |
| Response | n/N | 13/40 | 6/53 | p = 0.0185 |
| | % | 32.5 | 11.3 | |

n = number of subjects in response/remission,
N = number of subjects,

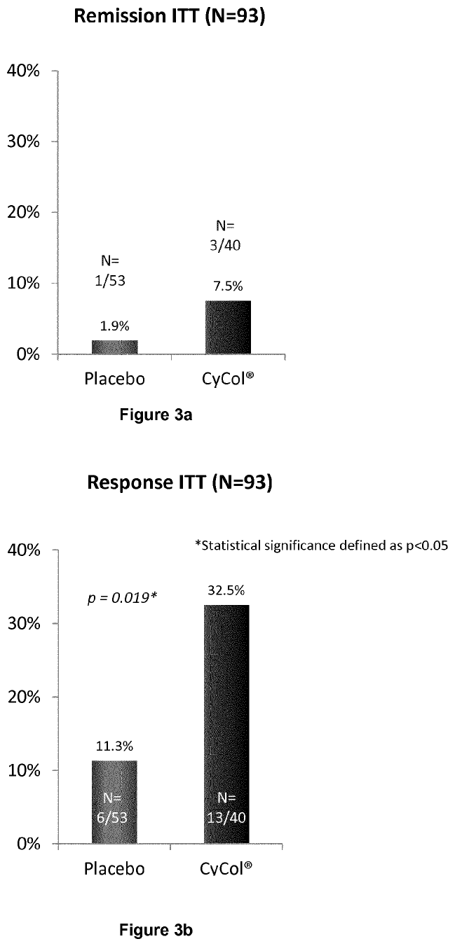

The data shown in Table 8 is represented in a chart in FIGS. 3a and 3b.

There is a numerically higher rate of remission and response in the CyCol® treatment group compared to the placebo group. The difference between the CyCol® and placebo rate of response is statistically significant. The difference between the rate of remission in the placebo and CyCol® treatment group (Δ) is 21.2.

### Example 4: Remission and Response in Subjects with Mild-Moderate Ulcerative Colitis being Medicated Concurrently with 5-ASA Only

In the ITT population following the Alternative Analysis Convention, there were 71 subjects classified into a population subgroup being concurrently treated with only 5-ASA. The remission rate and response rate for CyCol® and placebo therapy for this population subgroup are shown in Table 9 for this subject subset.

TABLE 9

| | | |
|---|---|---|
| | | Fisher's exact |
| CyCol ® | Placebo | test |

| | | | | |
|---|---|---|---|---|
| Remission | n/N | 7/34 | 2/37 | p = 0.0771 |
| | % | 20.6 | 5.4 | |
| Response | n/N | 12/34 | 4/37 | p = 0.0217 |
| | % | 35.3 | 10.8 | |

n = number of subjects in response/remission,
N = number of subjects,

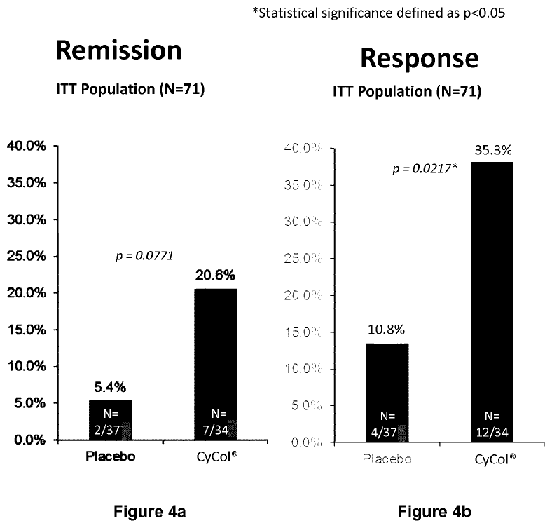

The data shown in Table 9 is represented in FIGS. 4a and 4b.

There is a numerically higher rate of remission and response in the CyCol® treatment group compared to the placebo group. The difference between the CyCol® and placebo rate of response is statistically significant. The Δ for the response value is 24.5.

### Example 5: Remission and Response in Subjects with Moderate Ulcerative Colitis being Medicated Concurrently with 5-ASA Only

In the ITT population, following the New Alternative Analysis Convention for clinical remission and response, 54 subjects were characterised into a population subgroup as having moderate UC and being treated concurrently with only 5-ASA. The remission rate and response rate for CyCol® and placebo treatment for this population subgroup are shown in Table 10.

TABLE 10

| | | | | Fisher's exact |
|---|---|---|---|---|
| | | CyCol ® | Placebo | test |
| Remission | n/N | 3/24 | 1/30 | p = 0.312 |
| | % | 12.5 | 3.3 | |
| Response | n/N | 10/24 | 3/30 | p = 0.010 |
| | % | 41.7 | 10.0 | |

n = number of subjects in response/remission,
N = number of subjects,

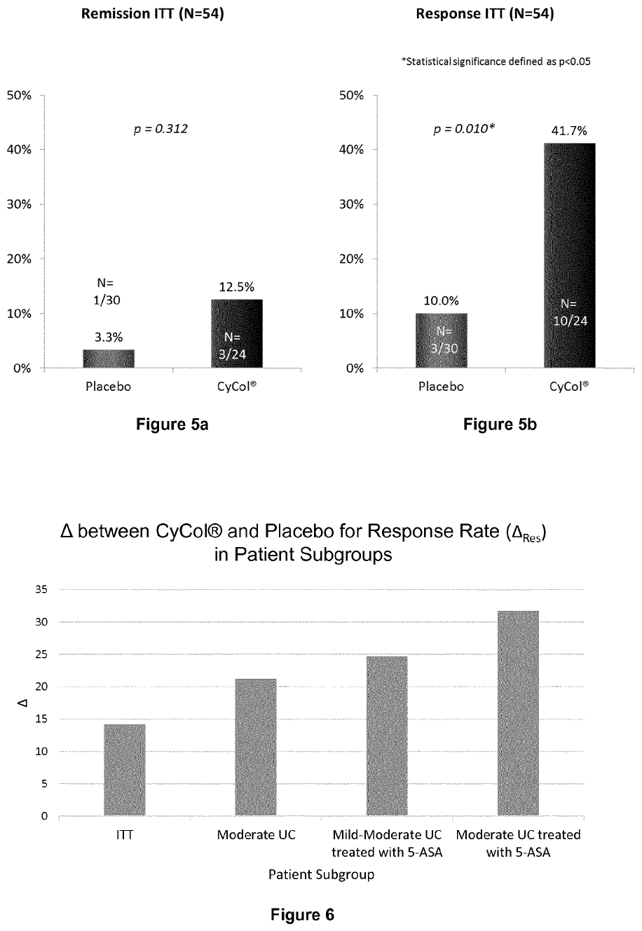

The data shown in Table 10 is represented in FIGS. 5a and 5b.

There is a numerically higher rate of remission and response in the CyCol® treatment group compared to the placebo group. The difference between the CyCol® and placebo rate of response is statistically significant. The Δ for the response value is 31.7.

### Example 6: Comparison of Δ_{Res} from Examples 2 to 5

Table 11 shows the Δ_{Res}—difference between the % of patients showing a clinical response for the CyCol® treatment group compared to the placebo group—for each of the subject subgroups for Examples 2 to 5. The data shown in Table 11 is represented in FIG. 6.

TABLE 11

| Example | Subject Subgroup | Δ_{Res} |
|---|---|---|
| 2 | ITT | 14.2 |
| 3 | Moderate UC | 21.2 |
| 4 | Mild/moderate UC treated | 24.5 |
| | with 5-ASA | |
| 5 | Moderate UC treated with | 31.7 |
| | 5-ASA | |

Across the entire subject population (ITT) and each of the population subgroups discussed in Example 3 to 5 the analysis of the study data showed a larger rate of response in the CyCol® treatment group compared to the placebo group. The Δ_{Res} was 14.2 for the entire subject population (ITT; Example 2), with subjects suffering from mild or moderate UC. The Δ_{Res} for the patient subgroup with moderate UC was higher than the Δ_{Res} for the entire subject population. Similarly, a higher Δ_{Res} was found for the subject subgroup suffering from moderate UC and being treated with 5-ASA compared to the Δ_{Res} for the subject subgroup suffering from mild/moderate UC and being treated with 5-ASA. This shows an improved efficacy for the CyCol® treatment in subjects with moderate UC compared to those with mild UC and an improved efficacy for the CyCol® treatment when administered concurrently to a 5-ASA treatment compared to subjects not being treated with 5-ASA.

### Example 7: Comparison of Δ_{Res} for CyCol® Therapy to Δ_{Res} Values of Approved Ulcerative Colitis Therapies

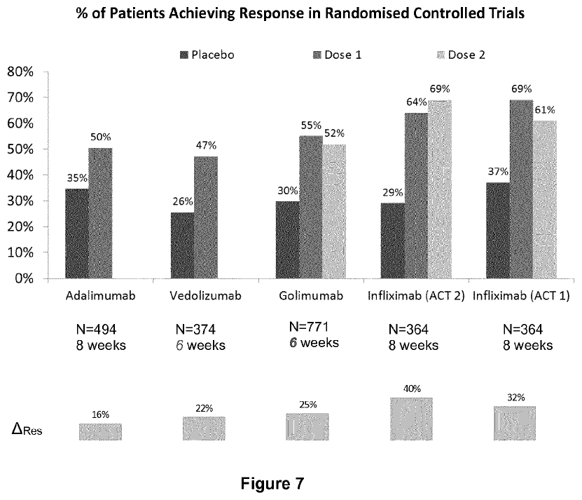

FIG. 7 shows the percentage of subjects achieving a clinical response in populations receiving placebo or an approved therapy for UC. FIG. 7 also shows the difference between the percentage of patients achieving a clinical response in populations receiving placebo and the approved therapy (Δ_{Res}) for each therapy. The Δ_{Res} values for the approved therapies in FIG. 7 range from 16% to 40%.

For a target UC population of CyCol® therapy, moderate UC being treated with 5-ASA, CyCol® exhibits a greater Δ_{Res} than three of the five approved therapies and exhibits a comparable Δ_{Res} for the two other approved therapies (Infliximab Active Ulcerative Colitis Trial 1 (ACT1) and Infliximab Active Ulcerative Colitis Trial 2 (ACT2)).

In addition, the present CyCol® study was run for 4 weeks, whereas the trials for which the results are shown in FIG. 7 were run for 6 or 8 weeks.

### Example 8: Remission and Response in Patients with Mild-Moderate Ulcerative Colitis and being Medicated Concurrently with 5-ASA and Steroids

In the subject population, following the New Alternative Analysis Convention, there was a subgroup of 10 subjects being treated concurrently with both 5-ASA and steroids. The remission rate and response rate for CyCol® and placebo therapy for this population subgroup are shown in Table 12.

TABLE 12

| | | | | Fisher's exact |
|---|---|---|---|---|
| | | CyCol ® | Placebo | test |
| Remission | n/N | 2/5 | 0/5 | p = 0.444 |
| | % | 40.0 | 0.0 | |
| Response | n/N | 3/5 | 0/5 | p = 0.1667 |
| | % | 60.0 | 0.0 | |

n = number of subjects in response/remission,
N = number of subjects,

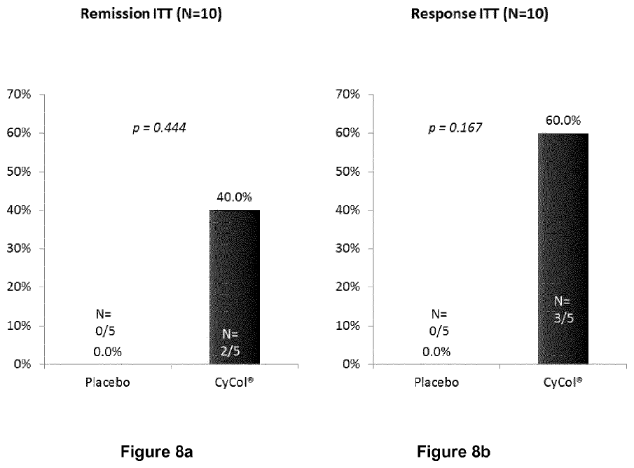

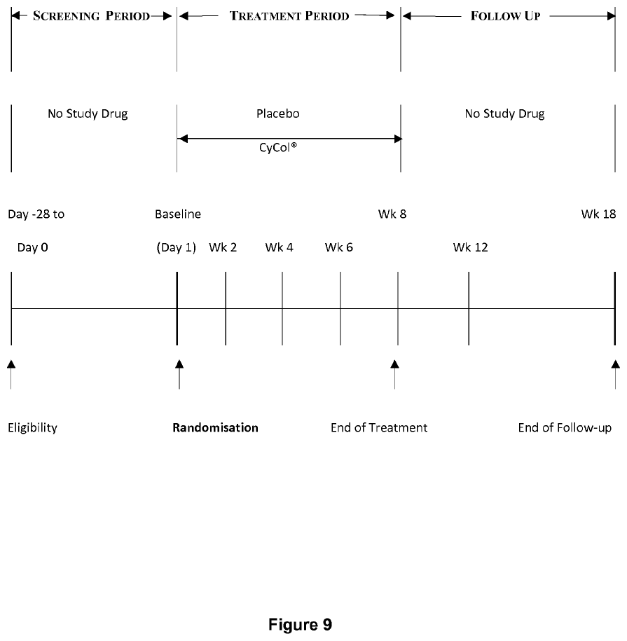

The data shown in Table 12 is represented in FIGS. 8a and 8b.

There is a numerically higher rate of remission and response in the CyCol® treatment group compared to the placebo group. The difference between the CyCol® and placebo rate of response is not statistically significant. The Δ for the response value is 60.

The absence of any observed improvement in the subjects being treated with 5-ASA and steroids, implies that the subjects might be refractory to steroids. In contrast, all of the CyCol® treatment group achieved either remission or response. Hence, such a steroid refractory patient subclass might benefit from the inclusion of CyCol® in their treatment regimen.

### Example 9: Evaluation of the Safety and Tolerability of the Minibeads of Example 1

Safety and tolerability were evaluated throughout the study by:

Adverse events (AEs),

Physical examination,

Vital signs,

Clinical laboratory (haematology, serum chemistry),

UC clinical assessment (determined by stool frequency, rectal bleeding, and mucosal appearance).

### Adverse Events (AE)

An AE is defined as any untoward medical occurrence in a subject administered a pharmaceutical product during the course of a clinical investigation. An AE can therefore be any unfavorable and unintended sign, symptom, or disease temporally associated with the use of an investigational product or study procedure, whether or not thought to be related to the investigational product.

Subjects were monitored from the time of informed consent until the end of the study for AEs.

Adverse events were recorded and the severity was graded according to the following definitions:

Mild: The subject experiences awareness of symptoms but these are easily tolerated or managed without specific treatment

Moderate: The subject experiences discomfort enough to cause interference with usual activity, and/or the condition requires specific treatment

Severe: The subject is incapacitated with inability to work or do usual activity, and/or the event requires significant treatment measures.

Action taken was categorized as none, concomitant medication given, non-drug therapy given, hospitalized or study drug discontinued and the event outcome at resolution or time of last follow-up will be recorded as event resolved, resolved with sequelae, ongoing, or death.

The relationship of the adverse event to the study drug was determined by the investigator according to the following criteria:

Not related: The event is most likely produced by other factors such as the subject's clinical condition, intercurrent illness, or concomitant drugs, and does not follow a known response pattern to the study drug, or the temporal relationship of the event to study drug administration makes a causal relationship unlikely

Possibly related: The event follows a reasonable temporal sequence from the time of drug administration, and/or follows a known response pattern to the study drug, but could have been produced by other factors such as the subject's clinical condition, intercurrent illness, or concomitant drugs

Probably related: The event follows a reasonable temporal sequence from the time of drug administration, and/or follows a known response pattern to the study drug, and cannot be reasonably explained by other factors such as the subject's clinical condition, intercurrent illness or concomitant drugs.

### Serious Adverse Events (SAE)

The “seriousness” of an adverse event was determined by the Investigator. An SAE was any AE occurring at any dose that results in any of the following outcomes:

Death

A life-threatening adverse drug experience

Results in inpatient hospitalization or prolongation of existing hospitalization

A persistent or significant disability/incapacity

A congenital anomaly/birth defect

Important medical events that may not result in death, be life threatening, or require hospitalization were considered an SAE when, based upon appropriate medical judgment, they may have jeopardized the subject and may have required medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

Elective hospitalizations or surgical procedures that were a result of a subject's preexisting condition(s) which had not worsened since receiving study drug, were not considered as SAEs. Examples may include, but are not limited to; pre-planned cholecystectomy for gallstones, joint replacement surgery, or diagnostic testing.

### Vital Signs

Vital signs (blood pressure, heart rate, respiratory rate, and temperature) were performed at the Screening, Day 0, Week 4, and Follow-up visits. Vital signs were measured at each visit after the subject has been sitting for at least 5 minutes.

### Ulcerative Colitis Clinical Assessment and DAI

UC clinical assessment was determined by stool frequency and rectal bleeding.

The score for each assessment (i.e. stool frequency and rectal bleeding) was based on patient recall over three days prior to study visit. The three day score for each assessment was added and then divided by three to obtain a total score for the DAI table.

Mucosal appearance was assessed and scored during the sigmoidoscopy with the total score being recorded in the DAI table at Baseline and Week 4.

### Safety Evaluation Data

The Safety population (SAF) consisted of 118 subjects, including the 117 subjects of the intention to treat population and the 1 non-eligible subject. The mean exposure to the study drug was 24.5 days (SD: 7.19) in the SAF overall. For the CyCol® group, it was 25.7 days (SD: 5.99) and for the placebo group 23.5 days (SD: 7.96).

The mean number of capsules taken was 70.9 (SD: 22.55) for the SAF overall (CyCol®: 74.9 [SD: 18.75], placebo: 67.2 [SD: 25.13]).

### Adverse Events

Overall, 94 subjects (79.7%) of the SAF had 238 treatment-emergent Adverse effects (TEAEs) with 262 symptoms according to MedDRA. For the occurrence of TEAEs in the individual treatment groups, refer to Table 14. The rate of subjects with at least 1 TEAE did not differ significantly between the treatment groups (p=0.6485).

TABLE 14

| | | CyCol ® | | Placebo | |
|---|---|---|---|---|---|
| | | (N = 53) | | (N = 65) | |
| | | N | (%) | N | (%) |
| Number of subjects with TEAE | | 41 | (77.4) | 53 | (81.5) |
| Number of symptoms (acc. to MedDRA) | | 117 | — | 145 | — |
| Number of episodes | | 107 | — | 131 | — |
| Causality assessment | Not related | 86 | (80.4) | 96 | (73.3) |
| | Possibly related | 21 | (19.6) | 34 | (26.0) |
| | Probably related | 0 | (0.0) | 1 | (0.8) |
| Intensity | Mild | 57 | (53.3) | 60 | (45.8) |
| | Moderate | 28 | (26.2) | 56 | (42.7) |
| | Severe | 22 | (20.6) | 15 | (11.5) |

N = number (of subjects)

### Display of Adverse Events

Overall, the most frequently reported symptoms according to MedDRA preferred term (PT) were ‘Colitis ulcerative’ (N=26 [22.0%]), ‘Rectal haemorrhage’ (N=23 [19.5%]), and ‘Frequent bowel movements’ (N=21 [17.8%]).

The number of subjects with the most frequently reported (occurrence ≥5% overall) TEAEs by PT are shown in Table 15 divided by treatment group. ‘Colitis ulcerative’ was documented for a higher rate of subjects in the placebo group compared to the CyCol® group. Of note is the absence from Table 15 of paresthesia, a tingling, tickling or burning sensation of a person's skin, that is a common adverse event identified in clinical trials with Sandimmune™ and Neoral™ (see Neoral™ Soft Gelatin Capsules (cyclosporin capsules, USP) MODIFIED, Neoral™ Oral Solution (cyclosporin oral solution, USP) MODIFIED, Prescribing Information).

TABLE 15

| | |
|---|---|
| CyCol ® | Placebo |
| (N = 53) | (N = 65) |

| PT | N | (%) | N | (%) |
|---|---|---|---|---|
| Colitis ulcerative | 5 | (9.4) | 21 | (32.3) |
| Rectal haemorrhage | 10 | (18.9) | 13 | (20.0) |
| Frequent bowel movements | 9 | (17.0) | 12 | (18.5) |
| Condition aggravated | 9 | (17.0) | 11 | (16.9) |
| Headache | 10 | (18.9) | 7 | (10.8) |
| Abdominal pain | 8 | (15.1) | 3 | (4.6) |
| Nasopharyngitis | 4 | (7.5) | 5 | (7.7) |
| Nausea | 4 | (7.5) | 5 | (7.7) |

N = number (of subjects)

### Serious Adverse Events

No subject died in the course of this study.

Overall, for 10 subjects (8.5%) of the SAF 15 serious TEAEs with 16 symptoms were reported. All events were of moderate (N=4 [26.7%]) or severe (N=11 [73.3%]) intensity. The rate of subjects with at least 1 serious TEAE did not differ significantly between the treatment groups (p=0.7518).

14 serious TEAEs (93.3%) were assessed to be not related to the study drug.

1 serious TEAE (6.7%) documented for the placebo group (subject no. 1901) was judged to be possibly related to treatment. It was a severe exacerbation of UC, lasting from 24 Jul. 2010 to 30 Jul. 2010 and requiring hospitalization of the subject. As a consequence, the study drug was discontinued. The event resolved completely.

Other Significant Adverse Events

22 subjects (18.6%) of the SAF discontinued the study prematurely due to an AE: 6 subjects (11.3%) in the CyCol® and 16 subjects (24.6%) in the placebo group.

Vital Signs and other Physical Findings

Overall, no clinically significant changes between Baseline and Week 4 were found for vital signs or the body weight in the SAF. Table 16 shows the pre-post differences between Baseline and Week 4 for the two treatment groups, also not revealing any clinically significant change.

TABLE 16

| | CyCol ® | | | Placebo | | |
|---|---|---|---|---|---|---|
| | (N = 53) | | | (N = 65) | | |
| | Nₘᵢₛₛ | Mean | (SD) | Nₘᵢₛₛ | Mean | (SD) |
| Systolic blood pressure [mmHg] | 5 | 3.1 | (16.24) | 6 | 1.7 | (15.63) |
| Diastolic blood pressure [mmHg] | 5 | 1.1 | (10.26) | 6 | 0.2 | (11.00) |
| Heart rate [bpm] | 5 | −1.2 | (11.79) | 6 | −1.3 | (14.12) |
| Body temperature [° C.] | 6 | 0.03 | (0.624) | 10 | −0.15 | (0.496) |
| Respiratory rate [pm] | 6 | 0.0 | (2.21) | 7 | 0.2 | (1.35) |
| Body weight [kg] | 4 | −0.41 | (1.714) | 4 | −0.48 | (1.505) |

N = number (of subjects),
Nₘᵢₛₛ = number of missing values,
SD = standard deviation

### CONCLUSIONS

A considerable number of TEAEs was documented in this study but the majority of these were judged to be not related to the study medication. Additionally, the analysis by MedDRA PT showed, that most of the TEAEs were due to the underlying disease.

1 serious TEAE with probable relation to the study medication occurred in the placebo treatment group, but resolved completely after medication withdrawal.

Laboratory analyses (including pregnancy tests and faeces samples) showed no clinically significant abnormalities.

CyCol® met the safety endpoint with no difference between subjects receiving CyCol® and those receiving placebo. CyCol® was well tolerated; no safety concerns were raised. In addition, paresthesia, a common side effect associated with treatment by cyclosporin was not observed, providing a significant benefit to CyCol® treatment over other cyclosporin therapies.

### Example 10: Preparation of a Minibead Having an Opadry Subcoat and a Surelease/Pectin Coating

The minibead was generally prepared as described in Example 1. However, after forming the core and before coating the core with a mixture of Surelease® (an ethylcellulose dispersion) and Pectin a coating of a dispersion of Opadry White 20A28380 (supplied by Colorcon) was applied to the core following the procedure described below.

The minibead cores were loaded into a fluid bed coater (Wurster column) and coated with Opadry White 20A28380 (supplied by Colorcon Limited) as a dispersion. The processing parameters, such as inlet air temperature and inlet air volume, were adjusted to keep the minibead temperature between 40° C. and 42° C. until the required coating weight gain of 5% was reached. The resulting subcoated minibeads were dried for 5 minutes at 40° C. in the coater.

The subcoated cores were subsequently coated with the mixture of Surelease® and Pectin to a weight gain of 11.5% and processed as described in Example 1. This procedure provided cores with the composition shown in Table 17, the values being the weight percent of the total weight for each component.

TABLE 17

| Component | w/w % |
|---|---|
| Cyclosporin A | 9.2 |
| Miglyol 810 N | 3.9 |
| Transcutol HP | 14.0 |
| Kolliphor EL | 7.9 |
| SDS | 3.4 |
| Sorbitol | 4.9 |
| Gelatin | 42.1 |
| Opadry | 4.3 |
| Surelease | 10.1 |
| (Solids contents) | |
| Pectin | 0.2 |

### Example 11: Evaluation of the Efficacy of Minibeads in Subjects with Moderate to Severe Ulcerative Colitis

The efficacy of the oral modified release compositions comprising cyclosporin described herein, such as those of Example 10 may be evaluated by a study as described in the protocol below. The study may be a double-blind, placebo controlled study to evaluate the safety and efficacy of CyCol® (a composition comprising minibeads described herein such as Example 10) in Subjects with moderate to severe ulcerative colitis (UC) on background 5-aminosalicylic acids (5-ASA) and/or oral corticosteroid therapy in a routine clinical practice setting. Minibeads with a modified release composition different to that shown in Example 10 may be used in this study, for example any one of the compositions disclosed herein, including the minibeads may be used. The composition (for example minibeads) may comprise a subcoat, for example a subcoat of Opadry, or may be free of a subcoat. The minibeads of the study may comprise solubilised cyclosporin.

### Primary Objective:

The primary objective is to demonstrate the efficacy of CyCol® in inducing clinical remission in subjects with moderate to severe ulcerative colitis at Week 8.

Clinical remission is defined as a total Mayo score of 2 points or lower, with no individual sub score exceeding 1 point.

### Secondary Objectives:

The secondary objectives are to:

Evaluate the efficacy of CyCol® in inducing a clinical response after 8 weeks of treatment. Clinical response is defined as a decrease from baseline in the total Mayo score of ≥3 points, and at least 30%, with an accompanying decrease in the sub score for rectal bleeding of at least 1 point or an absolute sub score for rectal bleeding of 0 or 1.

Evaluate the efficacy of CyCol® in inducing mucosal healing after 8 weeks of treatment. Mucosal healing is defined as a reduction in endoscopy score of at least 1 point, or an absolute sub score for endoscopy of 0 or 1.

Evaluate the safety and tolerability of CyCol®. The safety and tolerability will be evaluated by Adverse Events (AEs), serious adverse events (SAEs), laboratory values, vital signs, physical examination, withdrawals due to AEs and concomitant medications.

### Investigational Plan

### Overview of Study Design and Dosing Regimen

This is a multicentre, randomized, double-blind, placebo controlled, parallel group study, consisting of two doses of CyCol® versus placebo in Subjects with moderate to severe UC, on background therapy of 5-ASA or steroids alone, or in combination.

Eligible Subjects will be randomized to receive oral CyCol® 75 mg BID, CyCol® 112.5 mg BID or placebo for 8 weeks. Randomisation will be stratified according to current corticosteroid exposure (yes/no) and disease severity (6-10, 11-12).

### Study Schema

The study may consist of three periods: up to 4 weeks for screening; an 8-week treatment period; and a 10-week follow up period as illustrated in FIG. 9.

### Subject Population and Selection

Approximately 300 adult men and women previously diagnosed with moderate to severe ulcerative colitis who are experiencing an inadequate clinical response to current treatment regimens, will be included in this study.

### Inclusion Criteria

To be eligible for this study, the Subjects must meet all the following criteria:
1. Male and female adult Subjects aged >18
2. Diagnosis of UC at least 3 months prior to Screening visit.
3. A combined stool frequency and rectal bleeding DAI score of ≥4, based on the Mayo scoring index, at the Screening visit
4. A DAI score of 6-12 inclusive, with a mucosal appearance score of ≥2, based on the Mayo scoring index, at Baseline visit
5. Concurrent treatment with at least 1 of the following:
   a. A stable dose of oral 5-ASA (e.g., mesalamine, or equivalent ≥2.4 g/day) for at least 14 days prior to Baseline. This dose is to be maintained for the duration of the treatment period
   b. A stable oral corticosteroid dose (≤30 mg/day or equivalent) for at least 14 days prior to Baseline
6. Patients with no known history of untreated, or inadequately treated latent or active TB infection
AND
Have a negative tuberculin test at screening visit or within 3 months of screening visit as determined by:
   a. a Mantoux Purified Protein Derivative (PPD) skin test OR
   b. QuantiFERON TB Gold (QFT Fold test)
AND
A chest radiograph taken within 3 months of Screening Visit, with negative findings for active TB infection.
7. Patients willing to practice adequate contraceptive methods to avoid pregnancy
8. Prepared to comply with study visits, treatment regimens and all required study procedures.
9. A signed and dated written patient consent form

### Exclusion Criteria

Subjects will be excluded from this study if they meet any of the following criteria:

### Disease

1. UC limited to rectum (ulcerative proctitis)
2. Evidence of fulminant colitis, toxic megacolon, or bowel perforation
3. A diagnosis of Crohn's colitis, indeterminate colitis, ischemic colitis, NSAID-induced colitis or radiation colitis
4. Subjects with evidence of pathogenic bowel infection
5. Previous surgery for UC or, Subjects who are likely to require surgery for UC during the study.
6. Any histological evidence of mucosal dysplasia or bowel stricture

### Laboratory and Other Medical Conditions

1. Subjects with a current or recent history of severe, progressive or uncontrolled cardiac (including uncontrolled hypertension), renal, hepatic, haematological, gastrointestinal, metabolic, endocrine, pulmonary, cardiac or neurological disease or any other severe co-morbid or psychiatric condition determined by the Investigator.
2. Evidence of Haematopoietic Disorders by the following laboratory values
3. Hemoglobin levels <9.0 g/dL or hematocrit <30% at Screening visit or within the 3 months prior to Baseline.
4. An absolute white blood cell (WBC) count of <3.0×109/L (<3000/mm3) or, ANC of <1.2×109/L (<1200/mm3), at Screening visit or within the 3 months prior to baseline.
5. Thrombocytopenia, as defined by a platelet count <100×109/L (<100,000/mm3) at Screening visit or within the 3 months prior to baseline.
6. Subjects with evidence of total bilirubin, aspartate aminotransferase (AST) or alanine aminotransferase (ALT) more than 2 times the upper limit of normal at screening visit.
7. Subjects with a serum creatinine level of >2 mg/dl.
8. Malignancies or history of malignancy with the exception of adequately treated or excised non-metastatic basal cell carcinoma or squamous cell carcinoma of the skin.
9. Evidence of current systemic infection (clinically or with temperature >38° C.), or previous history of clinically significant infection within 3 months of Baseline, involving hospitalization or parental antimicrobial therapy or opportunistic infection and which is judged by the Investigator to have the potential for exacerbation by participation in the study.

### Drug-specific

1. Subjects receiving the following therapy(ies) prior to, or at any time during the study:
2. Intravenous corticosteroids use within 14 days prior to Screening or during the Screening Period
3. Treatment with intravenous or oral antibiotics for any reason within 14 days of Screening visit
4. Treatment with any therapeutic enema or suppository, other than required for sigmoidoscopy, within 14 days prior to Baseline visit
5. Immunomodulatory therapy (e.g. AZA/6-MP) within 14 days of Baseline visit
6. Previous use of infliximab or any anti-TNF agent within 56 days of Baseline visit
7. Treatment with any calcineurin inhibitor within 56 days of Baseline visit
8. Unwilling to refrain from taking St. John's Wort or any drugs or substances known to inhibit or induce cytochrome (CYP) P450 enzymes during the 8 week treatment period
9. Does not agree to avoid grapefruit or grapefruit juice during the treatment period
10. Known hypersensitivity to cyclosporin or any excipients contained in CyCol®
11. Is planning to receive or has received any investigational product within 30 days of Baseline visit

### General

1. History of alcohol or drug abuse in the year prior to Baseline visit
2. Subjects unwilling to delay either conception or initiation of breast feeding for at least 90 days after the last dose of study drug administered.
3. Any other condition in the opinion of the Investigator or Sponsor that would make the Subject unsuitable for inclusion in the study

### Treatments

The study described in Example 2 in mild to moderate ulcerative colitis evaluated a once a day daily dose of 75 mg of CyCol® (3×25 mg capsules). This dose was approximately equivalent to 1 mg/kg dose in a person of average weight (70 kg).

In this study, a 37.5 mg CyCol® capsule may be tested as a 75 mg and 112.5 mg BID dose, which is approximately equivalent to 1 mg/kg and 1.5 mg/kg BID in an average 70 kg person. The study may also be run with candidates receiving 1, 2 or 4 capsules at least once daily. In addition the capsules may contain any amount of cyclosporin, for example from 35 mg to 40 mg.

At the Baseline Visit (Day 1) the first dose of study drug will be administered by the Investigator, or designee, at the clinic visit. If the clinic visit occurs in the morning, the Subject should be instructed to take their evening dose at home as described below; otherwise, the Subject should be instructed to take their next dose the following morning.

Subjects should be instructed to take 3 capsules once in the morning and once in the evening by mouth each day. All 3 capsules should be taken together at least 30 minutes before or, 2 hours after food for the 8 week study duration. The capsules should not be chewed, opened, or mixed with food or drink.

The clinical trial protocol above may be adapted to study alternative dosage regimens of the oral modified release compositions described herein, for example the following dosage regimens of the oral modified release compositions are contemplated:

37.5 mg cyclosporin once daily;
37.5 mg cyclosporin twice daily;
75 mg cyclosporin once daily;
75 mg cyclosporin twice daily;
150 mg cyclosporine once daily; or
150 mg cyclosporine twice daily.

1-131. (canceled)

132. A method for the treatment of moderate or severe ulcerative colitis to induce remission of the ulcerative colitis, the method comprising orally administering to the patient a therapeutically active amount of an oral modified release composition comprising cyclosporin, wherein the patient is treated concurrently with a biological therapy suitable for use in the treatment of ulcerative colitis.

133. The method of claim 132, wherein the biological therapy is an anti-TNF therapy or an integrin inhibitor therapy.

134. The method of claim 132, wherein the biological therapy is selected from infliximab, adalimumab, golimumab or vedolizumab.

135. The method of claim 132, further comprising a maintenance therapy to maintain the ulcerative colitis in remission, the maintenance therapy comprising orally administering to the patient a therapeutically active amount of the oral modified release composition comprising cyclosporin, wherein the patient is treated concurrently with a biological therapy suitable for use in the treatment of ulcerative colitis.

136. The method of claim 132, further comprising a maintenance therapy to maintain the ulcerative colitis in remission, the maintenance therapy comprising orally administering to the patient a therapeutically active amount of the oral modified release composition comprising cyclosporin alone.

137. The method of claim 132, further comprising administering to the patient a therapeutically effective amount of (i) an aminosalicylate or (ii) a steroid or (iii) a combination of an aminosalicylate and a steroid.

138. The method of claim 132, wherein the oral modified release composition comprising cyclosporin is orally administered to the patient to provide a total daily dose of cyclosporin of from about 1 mg to about 500 mg.

139. The method of claim 132, wherein the oral modified release composition comprising cyclosporin is orally administered to the patient in a dose of 37.5 mg, 75 mg or 150 mg once or twice per day.

140. The method of claim 132, wherein the oral modified release composition comprising cyclosporin comprises a matrix and cyclosporin;
wherein the matrix comprises a polymer matrix selected from a water-permeable polymer, a water-swellable polymer, a water-soluble polymer, a hydrogel-forming polymer or a biodegradable polymer.

141. The method of claim 140, wherein the oral modified release composition comprising cyclosporin comprises cyclosporin and a modified release coating to control or modulate release of the cyclosporin from the composition.

142. The method of claim 140, wherein the modified release coating comprises a polymeric material and the polymeric material is selected from a controlled release polymer, a sustained release polymer, an enteric polymer, a pH independent polymer, a pH dependent polymer, a polymer specifically susceptible to degradation by bacterial enzymes in the gastrointestinal tract, or a combination of two or more such polymers.

143. The method of claim 140, wherein the modified release coating comprises ethyl cellulose.

144. The method of claim 132, wherein the oral modified release composition comprising cyclosporin comprises cyclosporin, a first coating and a second coating outside the first coating; and wherein
the first coating comprises a water-soluble cellulose ether or a water-soluble derivative of a cellulose ether; and
the second coating comprises a modified release coating comprising a pH independent polymer.

145. The method of claim 144, wherein the first coat comprises one or more water-soluble cellulose ethers selected from an alkyl cellulose; a hydroxyalkyl cellulose; a hydroxyalkyl alkyl cellulose; or a carboxyalkyl cellulose.

146. The method of claim 144, wherein the first coating comprises hydroxypropylmethyl cellulose.

147. The method of claim 144, wherein the first coating is present in an amount corresponding to a weight gain due to the first coating of from 1% to 20% by weight based upon the weight of the composition prior to applying the first coating.

148. The method of claim 144, wherein the second coating is present in an amount corresponding to a weight gain of the composition due to the second coating of from 5% to 20%, based upon the weight of the composition prior to applying the second coating.

149. The method of claim 144, wherein the first coating comprises hydroxypropylmethyl cellulose and the second coating comprises ethyl cellulose.

150. The method of claim 144, wherein the composition comprises a core, the first coating is outside the core and the second coating is outside the first coating, wherein the core comprises a hydrogel forming polymer matrix and cyclosporin.

151. The method of claim 150, wherein the core is in the form of a solid colloid, the colloid comprising a continuous phase and a disperse phase, wherein the continuous phase comprises the hydrogel forming polymer.

152. The method of claim 151, wherein the cyclosporin is comprised in the disperse phase.

153. The method of claim 151, wherein the disperse phase comprises a hydrophobic excipient and optionally a solvent miscible therewith, optionally wherein the cyclosporin is soluble in the disperse phase.

154. The method of claim 151, wherein the disperse phase comprises a disperse phase selected from caprylic/capric triglyceride; caprylic/capric/linoleic triglyceride;
caprylic/capric/succinic triglyceride; or propylene glycol dicaprylate/dicaprate.

155. The method of claim 151, wherein the disperse phase comprises an oil phase comprising a surfactant with an HLB of from 0 to 10.

156. The method of claim 151, wherein the disperse phase comprises an oil phase which represents 10-85% by dry weight of the core.

157. The method of claim 150, wherein the core further comprises a surfactant, optionally wherein the surfactant is an anionic surfactant or a non-ionic surfactant or a combination thereof.

158. The method of claim 150, wherein the core comprises a surfactant present in at least the continuous phase, the surfactant having an HLB value of at least 10.

159. The method according to claim 158, wherein the surfactant in the continuous phase is at least one surfactant selected from fatty acid salts, alkyl sulfates or bile salts.

160. The method of claim 151, wherein the disperse phase comprises a surfactant with an HLB value in the range of from 1 to 15.

161. The method of claim 151, wherein the colloid comprises a continuous phase comprising a hydrogel forming polymer; and a disperse phase comprising cyclosporin and an oil phase, the oil phase comprising an oil and one or more surfactants, wherein the surfactants have an HLB in the range 0-10.

162. The method of claim 161, wherein the surfactant comprises a surfactant selected from: fatty acid glycerides, polyethylene glycol fatty acid esters, propylene glycol fatty acid esters, fatty acid lactic acid ester, sucrose fatty acid esters, sorbitan fatty acid esters, polyethylene glycol fatty alcohol ethers, ethylene oxide-propylene oxide block co-polymers or polyoxyethylene ethers.

163. The method of claim 161, wherein the surfactant is selected from sorbitan trioleate, sorbitan monopalmitate; polyglyceryl-3 dioleate or oleoyl macrogol-6 glycerides.

164. The method of claim 150, wherein the hydrogel forming polymer matrix comprises a hydrocolloid, a non-hydrocolloid gum or chitosan.

165. The method of claim 150, wherein the hydrogel forming polymer matrix comprises gelatin, agar, a polyethylene glycol, starch, casein, chitosan, soya bean protein, safflower protein, alginates, gellan gum, carrageenan, xanthan gum, phthalated gelatin, succinated gelatin, cellulosephthalate-acetate, oleoresin, polyvinylacetate, hydroxypropyl methyl cellulose, polymerisates of acrylic or methacrylic esters and polyvinylacetate-phthalate and any derivative of any of the foregoing hydrogel forming polymers; or a mixture of two or more of any of the foregoing hydrogel forming polymers.

166. The method according to claim 151, wherein the disperse phase of the core comprises:
cyclosporin;
a medium chain mono- di- or tri-glyceride;
a non-ionic surfactant; and
a solvent;
and wherein the continuous phase of the core comprises:
a hydrogel forming polymer matrix which comprises a hydrocolloid selected from carrageenan, gelatin, agar and pectin, or a combination thereof;
optionally a plasticiser; and
an anionic surfactant.

167. The method of claim 132, wherein the oral modified release composition comprising cyclosporin is in the form of a multiplicity of minibeads.

168. The method of claim 167, wherein the largest cross-sectional dimension of the minibead is from 0.1 to 5 mm.

169. The method of claim 132, wherein the oral modified release composition comprising cyclosporin releases less than 15% of the cyclosporin after 2 hours; releases 10% to 40% of the cyclosporin at 4 hours; and releases from about 25% to 70% of the cyclosporin between 4 hours and 12 hours, when measured in a two stage dissolution test using a USP Apparatus II with a paddle speed of 75 rpm and a dissolution medium temperature of 37° C.; wherein for the first 2 hours of the dissolution test the dissolution medium is 750 ml of 0.1 N HCl, and at 2 hours 250 ml of 0.2M tribasic sodium phosphate containing 2% SDS is added to the dissolution medium and the pH is adjusted to pH 6.8.
